(19) United States
(12) Patent Application Publication
(10) Pub. No.: US 2025/0066512 A1
(43) Pub. Date: Feb. 27, 2025

(54) MODIFIED POLYSACCHARIDE POLYMERS AND RELATED COMPOSITIONS AND METHODS THEREOF

(71) Applicant: SIGILON THERAPEUTICS, INC., Cambridge, MA (US)

(72) Inventors: Matthew Buchanan, Atkinson, NH (US); Omar De Paolis, Boston, MA (US); Weiheng Wang, Bedford, MA (US); Christopher P. Hencken, Boston, MA (US)

(21) Appl. No.: 18/724,811

(22) PCT Filed: Dec. 30, 2022
(86) PCT No.: PCT/US2022/054362

§ 371 Date: Jun. 27, 2024

Related U.S. Application Data
(60) Provisional application No. 63/295,268, filed on Dec. 30, 2021.

(51) Int. Cl. C08B 37/00; A61K 9/00; A61K 9/48; A61K 47/61; A61L 31/04; A61L 31/14; A61L 31/16

(52) U.S. Cl. CPC C08B 37/0084; A61K 9/0024; A61K 9/4816; A61K 47/61; A61L 31/042; A61L 31/145; A61L 31/16; A61L 2300/412; A61L 2300/62; A61L 2400/18

(57) ABSTRACT

Described herein are polysaccharide polymers comprising a saccharide moiety modified with a hydroxyl-modifying agent (e.g., a saccharide monomer of Formula (I)), as well as related compositions, hydrogels, implantable elements, and methods of use thereof.

### CLAIM OF PRIORITY

This application claims priority to U.S. Patent Application No. 63/295,268, filed Dec. 30, 2022, the entire contents of which is incorporated herein by reference in its entirety.

### BACKGROUND

The function of implanted devices depends in large part on the biological immune response pathway of the recipient (Anderson et al., Semin. Immunol. 20:86-100 (2008); Langer, Adv. Mater. 21:3235-3236 (2009)). Modulation of the immune response may impart a beneficial effect on the fidelity and function of these devices. As such, there is a need in the art for new compounds, compositions, and devices that achieve this goal.

### SUMMARY

Described herein are polymers comprising certain hydroxyl group modifications, as well as related compositions and methods thereof. In one aspect, the present disclosure features a polysaccharide polymer comprising a saccharide monomer bearing a hydroxyl moiety, wherein the saccharide monomer comprises a hydroxyl-modifying agent covalently bound to the hydroxyl moiety. In an embodiment, the saccharide monomer bears a plurality of hydroxyl moieties, wherein the saccharide monomer comprises a hydroxyl-modifying agent covalently bound to a hydroxyl moiety. In another aspect, the present disclosure features a polysaccharide polymer comprising a saccharide monomer bearing a hydroxyl moiety, wherein the saccharide monomer comprises a hydroxyl-modifying agent in the place of the hydroxyl moiety. In some embodiments, the saccharide monomer is selected from glucose, galactose, mannose, allose, altrose, talose, idose, gulose, fructose, ribose, arabinose, lyxose, xylose, rhamnose, glucuronic acid, galacturonic acid, mannuronic acid, and guluronic acid. In an embodiment, the polysaccharide polymer is selected from hyaluronate, alginate, cellulose, chitosan, chitin, amylose, dextran, starch, glycogen, chondroitin, and pectin. In an embodiment, the hydroxyl-modifying agent is a nitrogen-containing hydroxyl-modifying agent. In an embodiment, the hydroxyl-modifying agent comprises an amine or an amide.

In another aspect, the present disclosure features a polysaccharide polymer comprising a saccharide monomer, wherein the saccharide monomer has a structure of Formula (I):

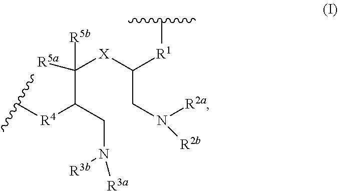

or a pharmaceutically acceptable salt thereof, wherein the variables X, R¹, R^{2a}, R^{2b}, R^{3a}, R^{3b}, R⁴, R^{5a}, R^{5b} and subvariables thereof are defined herein. In some embodiments, the saccharide monomer of Formula (I) or a pharmaceutically acceptable salt thereof has the structure of Formulas (I-a), (I-b), (I-c), (I-d), (I-e), (I-f), (I-g), (I-g), (I-h), (I-i), (I-j), (I-k), (I-l), or (I-m). In some embodiments, the saccharide monomer of Formula (I) or a pharmaceutically acceptable salt is shown in Table 1 herein. In an embodiment, the polysaccharide polymer is an alginate, chitosan, dextran, or hyaluronate. In an embodiment, the polysaccharide polymer is an alginate. In an embodiment, X is O. In an embodiment, R¹ and R⁴ are each independently O. In an embodiment, one of R^{5a} and R^{5b} is independently hydrogen and the other of R^{5a} and R^{5b} is independently C(O)R, wherein R is OH or NHR′. In an embodiment, R is hydrogen, an afibrotic compound, or a peptide.

In another aspect, the present disclosure features an alginate comprising a saccharide monomer, wherein the saccharide monomer has a structure of Formula (I):

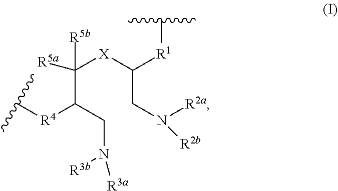

or a pharmaceutically acceptable salt thereof, wherein the variables X, R¹, R^{2a}, R^{2b}, R^{3a}, R^{3b}, R⁴, R^{5a}, R^{5b} and subvariables thereof are defined herein. In some embodiments, the saccharide monomer of Formula (I) or a pharmaceutically acceptable salt thereof has the structure of Formulas (I-a), (I-b), (I-c), (I-d), (I-e), (I-f), (I-g), (I-g), (I-h), (I-i), (I-j), (I-k), (I-l), or (I-m). In some embodiments, the saccharide monomer of Formula (I) or a pharmaceutically acceptable salt is shown in Table 1 herein. In an embodiment, the polysaccharide polymer is an alginate. In an embodiment, X is O. In an embodiment, R¹ and R⁴ are each independently O. In an embodiment, one of R^{5a} and R^{5b} is independently hydrogen and the other of R^{5a} and R^{5b} is independently C(O)R, wherein R is OH or NHR′. In an embodiment, R is hydrogen, an afibrotic compound, or a peptide.

In another aspect, the present disclosure features a hydrogel comprising a polysaccharide polymer (e.g., alginate) described herein, as well as an implantable element (e.g., a device or material) comprising the same. In some embodiments, the hydrogel or implantable element comprises a cell. Exemplary cell types include epithelial cells, endothelial cells, fibroblasts, keratinocytes, islet cells, and stem cells (e.g., iPSCs or MSCs). In some embodiments, the hydrogel or implantable element comprises an epithelial cell, e.g., a retinal pigment epithelial cell (RPE cell). In some embodiment, the hydrogel or implantable element comprises an islet cell. In some embodiments, the hydrogel or implantable element comprises a stem cell (e.g., an iPSC or MSC). In some embodiments, the hydrogel or implantable element comprises an engineered cell (e.g., an engineered epithelial cell, e.g., an engineered RPE cell, an engineered islet cell, or an engineered stem cell).

In some embodiments, the cell (e.g., an engineered cell) produces a substance, e.g., a therapeutic agent. Exemplary therapeutic agents include a nucleic acid (e.g., an RNA or DNA), protein (e.g., a hormone, enzyme, antibody, antibody fragment, antigen, or epitope), small molecule, lipid, drug, vaccine, or any derivative thereof. For example, an implantable element may comprise an engineered cell capable of producing a protein (e.g., an enzyme, blood clotting factor (e.g., Factor VIII protein) or hormone (e.g., insulin)).

In another aspect, the present disclosure features a method of providing a substance (e.g., a therapeutic agent) to a subject, comprising administering to the subject a hydrogel or implantable element comprising (i) a polysaccharide polymer (e.g., alginate) comprising a saccharide monomer of Formula (I), as described herein, and (ii) a cell capable of producing the substance (e.g., therapeutic agent). In some embodiments, the substance is a therapeutic agent, e.g., a protein (e.g., an enzyme, antibody, blood clotting factor (e.g., a Factor VIII protein) or hormone (e.g., insulin)).

In another aspect, the present disclosure features a method of treating a disease, disorder, or condition in a subject with a therapeutic agent that is capable of treating the disease, disorder or condition, the method comprising administering to the subject a hydrogel or implantable element comprising (i) a polysaccharide polymer (e.g., alginate) comprising a saccharide monomer of Formula (I), as described herein, and (ii) a cell capable of producing the therapeutic agent. In some embodiments, the disorder is a blood clotting disorder (e.g., Hemophilia A), a lysosomal storage disorder (e.g., Fabry Disease, MPS I), an endocrine disorder, diabetes, or a neurodegenerative disease.

In some embodiments, the method or providing a substance or method of treating comprises reducing the foreign body response to the administered implantable element (e.g., minimizing the formation of pericapsular fibrotic overgrowth (PFO) on the implantable element).

In any and all aspects of the disclosure, in some embodiments the polysaccharide polymer described herein, e.g., comprising a saccharide monomer of Formula (I), or a hydrogel or implantable element (e.g., device or material) thereof, is not a polysaccharide polymer, hydrogel, or implantable element described in any one of WO2012/112982, WO2012/167223, WO2014/153126, WO2016/187225, WO2016/019391, WO2017/075630, WO 2017/075631, WO 2018/067615, WO 2019/169333, and US 2016-0030359.

The details of one or more embodiments of the invention are set forth herein. Other features, objects, and advantages of the invention will be apparent from the Detailed Description, the FIGURES, the Examples, and the Claims.

### DETAILED DESCRIPTION

The present disclosure provides a polysaccharide polymer comprising a saccharide monomer bearing a hydroxyl moiety modified with a hydroxyl-modifying agent, as well as related compositions, and methods of making and use thereof. In an embodiment, the saccharide monomer comprises a hydroxyl-modifying agent covalently bound to a hydroxyl moiety. In an embodiment, polysaccharide polymer comprising, e.g., saccharide monomer of Formula (I), and hydrogels and implantable elements (e.g., devices and materials) comprising the same, as well as related compositions and methods of use thereof. In particular, the polysaccharide polymers, hydrogels, and implantable elements described herein may be used in methods for the prevention and treatment of a disease, disorder or condition in a subject. In some embodiments, the polysaccharide polymers comprising a saccharide monomer of Formula (I), and hydrogels and implantable elements comprising the same, as well as pharmaceutically acceptable salts, solvates, hydrates, tautomers, stereoisomers, isotopically labeled derivatives thereof, are capable of mitigating the immune response in a subject.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“About”, when used herein to modify a numerically defined parameter (e.g., a physical description of a polymer or implantable element as described herein, such as diameter, sphericity, number of cells in a particle (e.g., hydrogel), the number of particles in a preparation), means that the parameter may vary by as much as 15% above or below the stated numerical value for that parameter. For example, an implantable element defined as having a mean diameter of about 1.5 millimeters (mm) and encapsulating about 5 million (M) cells may have a mean diameter of 1.275 to 1.725 mm and may encapsulate about 4.25 M to 5.75 M cells. In some embodiments, the term “about” means that the parameter may vary by as much as 10% or 5% above or below the stated numerical value for that parameter.

“Acquire” or “acquiring”, as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., fluorescence microscope to acquire fluorescence microscopy data.

“Administer”, “administering”, or “administration”, as used herein, refer to implanting, absorbing, ingesting, injecting, disposing or otherwise introducing into a subject an entity described herein (e.g., a polysaccharide polymer described herein, or a hydrogel or implantable element comprising the same, including a hydrogel or implantable element encapsulating cells), or a composition comprising said particles), or providing the entity to a subject for administration. “Afibrotic”, as used herein, means a compound or material that mitigates at least one aspect of the foreign body response (FBR) to an implant comprising the compound or material, e.g., minimizes the formation of pericapsular fibrotic overgrowth (PFO) on the implant. For example, the FBR in a biological tissue or tissue fluid that is induced by implant into that tissue or tissue fluid of a polysaccharide polymer or device (e.g., hydrogel or implantable element) comprising an afibrotic compound (e.g., a hydrogel comprising a polysaccharide polymer covalently modified with a compound listed in Table 1) occurs in a lower amount, or at a later time, than the FBR induced by implantation of an afibrotic-null reference polymer or device, i.e., lacks any afibrotic compound, but otherwise has substantially the same composition (e.g., hydrogel capsule formed from the same non-modified polymer, and having substantially the same shape and size). In an embodiment, the degree of the FBR is assessed by the immunological response in the tissue or tissue fluid containing the implanted device (e.g., hydrogel capsule), which may include, for example, protein adsorption, macrophages, multinucleated foreign body giant cells, eosinophils, neutrophils, T cells, B cells, fibroblasts, and angiogenesis, using assays known in the art, e.g., as described in WO 2017/075630, or using one or more of the assays/methods described Vegas, A., et al., Nature Biotechnol (supra), (e.g., subcutaneous cathepsin measurement of implanted capsules, Masson's trichrome (MT), hematoxylin or eosin staining of tissue sections, quantification of collagen density, cellular staining and confocal microscopy for macrophages (CD68 or F4/80), granulocytes (Siglec-F, Ly-6G), myofibroblasts (alpha-muscle actin, SMA) or general cellular deposition, quantification of 79 RNA sequences of known inflammation factors and immune cell markers, or FACS analysis for macrophage and neutrophil cells on retrieved devices (e.g., capsules) after 14 days in the intraperitoneal space of a suitable test subject, e.g., an immunocompetent mouse. In an embodiment, the FBR is assessed by measuring the levels in the tissue or tissue fluid containing the implant of one or more biomarkers of immune response, e.g., cathepsin, TNF-α, IL-13, IL-6, G-CSF, GM-CSF, IL-1, IL-4, IL-5, CCL2, CCL4, TIMP-1. In some embodiments, the FBR is assessed by examining the amount of PFO on the implant (e.g., hydrogel capsule) at one or more times following the administration to suitable test subjects (e.g., immunocompetent mice); this assessment can be done using assays known in the art, e.g., any of the assays described in this definition. In some embodiments, an aspect of the FBR (e.g., PFO) induced by a modified polymer or device of the invention (e.g., a hydrogel capsule comprising an afibrotic compound described herein disposed on its outer surface), is at least about 80%, about 85%, about 90%, about 95%, about 99%, or about 100% lower than, or occurs at least about 10%, about 20%, about 40% or about 50% later than, the same FBR aspect induced by an afibrotic-null reference polymer or device. In some embodiments, the FBR (e.g., level of a biomarker(s)) is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer.

“Cell,” as used herein, refers to an engineered cell or a cell that is not engineered.

“Effective amount” as used herein refers to an amount of a compound, modified polymer, or implantable element described herein, e.g., further comprising a cell, e.g., an engineered cell, or an agent, e.g., a therapeutic agent, produced by a cell, e.g., an engineered cell, sufficient to mitigate or elicit a biological response, e.g., minimize an immune response, or to treat a disease, disorder, or condition. As will be appreciated by those of ordinary skill in this art, the effective amount may vary depending on such factors as the desired biological endpoint, the pharmacokinetics of the therapeutic agent, composition or implantable element, the condition being treated, the mode of administration, and the age and health of the subject. An effective amount encompasses therapeutic and prophylactic treatment. For example, to reduce the foreign body response (e.g., PFO) induced by an implantable element, a compound described herein may be disposed on the surface of the implantable element in an amount effective to reduce the PFO or stop the growth or spread of fibrotic tissue on or near the implantable element.

An “endogenous nucleic acid” as used herein, is a nucleic acid that occurs naturally in a subject cell.

An “endogenous polypeptide,” as used herein, is a polypeptide that occurs naturally in a subject cell.

“Engineered cell,” as used herein, is a cell having a non-naturally occurring alteration, and typically comprises a nucleic acid sequence (e.g., DNA or RNA) or a polypeptide not present (or present at a different level than) in an otherwise similar cell under similar conditions that is not engineered (an exogenous nucleic acid sequence). In an embodiment, an engineered cell comprises an exogenous nucleic acid (e.g., a vector or an altered chromosomal sequence). In an embodiment, an engineered cell comprises an exogenous polypeptide. In an embodiment, an engineered cell comprises an exogenous nucleic acid sequence, e.g., a sequence, e.g., DNA or RNA, not present in a similar cell that is not engineered. In an embodiment, the exogenous nucleic acid sequence is chromosomal, e.g., the exogenous nucleic acid sequence is an exogenous sequence disposed in endogenous chromosomal sequence. In an embodiment, the exogenous nucleic acid sequence is chromosomal or extra chromosomal, e.g., a non-integrated vector. In an embodiment, the exogenous nucleic acid sequence comprises an RNA sequence, e.g., an mRNA. In an embodiment, the exogenous nucleic acid sequence comprises a chromosomal or extra-chromosomal exogenous nucleic acid sequence that comprises a sequence which is expressed as RNA, e.g., mRNA or a regulatory RNA. In an embodiment, the exogenous nucleic acid sequence comprises a chromosomal or extra-chromosomal nucleic acid sequence, which comprises a sequence that encodes a polypeptide, or which is expressed as a polypeptide. In an embodiment, the exogenous nucleic acid sequence comprises a first chromosomal or extra-chromosomal exogenous nucleic acid sequence that modulates the conformation or expression of a second nucleic acid sequence, wherein the second amino acid sequence can be exogenous or endogenous. For example, an engineered cell can comprise an exogenous nucleic acid that controls the expression of an endogenous sequence. In an embodiment, an engineered cell comprises a polypeptide present at a level or distribution which differs from the level found in a similar cell that has not been engineered. In an embodiment, an engineered cell comprises a cell engineered to provide an RNA or a polypeptide. For example, an engineered cell may comprise an exogenous nucleic acid sequence comprising a chromosomal or extra-chromosomal exogenous nucleic acid sequence that comprises a sequence which is expressed as RNA, e.g., mRNA or a regulatory RNA. In an embodiment, an engineered cell comprises an exogenous nucleic acid sequence that comprises a chromosomal or extra-chromosomal nucleic acid sequence comprising a sequence that encodes a polypeptide, or which is expressed as a polypeptide. In an embodiment, an engineered cell comprises an exogenous nucleic acid sequence that modulates the conformation or expression of an endogenous sequence. In an embodiment, an engineered cell (e.g., RPE cell) is cultured from a population of stably transfected cells, or from a monoclonal cell line.

An “exogenous nucleic acid,” as used herein, is a nucleic acid that does not occur naturally in a subject cell, e.g., an engineered cell.

An “exogenous polypeptide,” as used herein, is a polypeptide that does not occur naturally in a subject cell, e.g., an engineered cell.

An “implantable element” as used herein, comprises a cell, e.g., a plurality of cells, e.g., a cluster of cells, wherein the cell or cells are entirely or partially disposed within an enclosing component (which enclosing component is other than a cell), e.g., the enclosing component comprises a non-cellular component. The term “implantable element” comprises a device or material described herein. In an embodiment, the implantable element inhibits an immune attack, or the effect of the immune attack, on the enclosed cell or cells. In an embodiment, the implantable element comprises a semipermeable membrane or a semipermeable polymer matrix or coating. An implantable element described herein comprises a polysaccharide polymer (e.g., an alginate) comprising a saccharide monomer having the structure of Formula (I), or a pharmaceutically acceptable salt thereof. An implantable element described herein may also comprise a polymer (e.g., a polysaccharide polymer, e.g., an alginate) or other material, optionally modified with another compound (e.g., an afibrotic compound or a peptide).

“Pericapsular fibrotic overgrowth” or “PFO”, as used herein, refers to a fibrotic cell layer that forms on part or all of a hydrogel or an implantable element as a result of the foreign body response to the implantable element.

“Polypeptide”, as used herein, refers to a polymer comprising amino acid residues linked through peptide bonds and having at least two, and in embodiments, at least 10, 100, or 200 amino acid residues.

“Prevention,” “prevent,” and “preventing” as used herein refers to a treatment that comprises administering or applying a therapy, e.g., administering a composition of implantable elements encapsulating cells (e.g., as described herein), prior to the onset of a disease, disorder, or condition to preclude the physical manifestation of said disease, disorder, or condition. In some embodiments, “prevention,” “prevent,” and “preventing” require that signs or symptoms of the disease, disorder, or condition have not yet developed or have not yet been observed.

A “replacement therapy” or “replacement protein” is a therapeutic protein or functional fragment thereof that replaces or augments a protein that is diminished, present in insufficient quantity, altered (e.g., mutated) or lacking in a subject having a disease or condition related to the diminished, altered or lacking protein. Examples are certain blood clotting factors in certain blood clotting disorders or certain lysosomal enzymes in certain lysosomal storage diseases. In an embodiment, a replacement therapy or replacement protein provides the function of an endogenous protein. In an embodiment, a replacement therapy or replacement protein has the same amino acid sequence of a naturally occurring variant, e.g., a wild type allele or an allele not associated with a disorder, of the replaced protein. In an embodiment, or replacement therapy or a replacement protein differs in amino acid sequence from a naturally occurring variant, e.g., a wild type allele or an allele not associated with a disorder, e.g., the allele carried by a subject, at no more than about 1, 2, 3, 4, 5, 10, 15 or 20% of the amino acid residues.

“Subject” as used herein refers to a human or non-human animal. In an embodiment, the subject is a human (i.e., a male or female, e.g., of any age group, a pediatric subject (e.g., infant, child, adolescent) or adult subject (e.g., young adult, middle-aged adult, or senior adult)). In an embodiment, the subject is a non-human animal, for example, a mammal (e.g., a primate (e.g., a cynomolgus monkey or a rhesus monkey)). In an embodiment, the subject is a commercially relevant mammal (e.g., a cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development.

A non-human animal may be a transgenic animal. “Treatment,” “treat,” and “treating” as used herein refers to one or more of reducing, reversing, alleviating, delaying the onset of, or inhibiting the progress of one or more of a symptom, manifestation, or underlying cause, of a disease, disorder, or condition. In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., considering a history of symptoms and/or in light of genetic or other susceptibility factors). Treatment may also be continued after symptoms have resolved, for example, to delay or prevent recurrence. In some embodiments, treatment comprises prevention and in other embodiments it does not.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₃), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”), 2 to 8 carbon atoms (“C₂-C₅ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen, phosporous, silicon, or sulfur atoms may optionally be oxidized, and the nitrogen heteroatom may optionally be quaternized. The heteroatom(s) 0, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆ alkylene, C₂-C₆ alkenylene, C₂-C₆ alkynylene, or C₁-C₆ heteroalkylene. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro [4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR^{C}R^{D}, wherein R^{C} and R^{D} are each independently hydrogen, C₁-C₁₂ alkyl, C₃-C₁₀ cycloalkyl, C₃-C₁₀ heterocyclyl, C₆-C₁₀ aryl, and C₅-C₁₀ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “hydroxyl” or “hydroxy” refers to the radical —OH.

As used herein, a “hydroxyl-modifying agent” is a moiety, e.g., a substance or compound, capable of binding to a material comprising a hydroxyl moiety (i.e., —OH), at the hydroxyl moiety position. For example, a hydroxyl-modifying agent may bind to a carbon atom covalently bound to a hydroxyl moiety. Exemplary hydroxyl-modifying agents include amines, esters, and thiols. The hydroxyl-modifying agent may bind covalently or non-covalently to the material comprising the hydroxyl moiety. To enable covalent binding of a hydroxyl-modifying agent to a material comprising a hydroxyl moiety, e.g., as described herein, the saccharide moiety comprising the hydroxyl moiety may be subjected to certain reactions to activate the hydroxyl moiety and/or the atoms surrounding the hydroxyl moiety, such as oxidation, reduction, or amination.

As used herein, a “saccharide” or “sugar” refers to a molecule comprising carbon, hydrogen, and oxygen atoms. In some embodiments, a saccharide further comprises another heteroatom (e.g., sulfur, phosphorus, or nitrogen). A saccharide may form a ring structure (e.g., a 4, 5, 6, 7, 8, 9-membered ring) or may be acyclic (e.g., linear). Exemplary saccharides include glucose, glucosamine, N-acetylglucosamine, glucuronic acid, galactose, galatosamine, N-acetylgalactosamine, galacturonic acid, mannose, mannosamine, N-acetylmannosamine, mannuronic acid, guluronic acid, idose, xylose, talose, fructose, and variants thereof.

As used herein, a “polysaccharide” refers to a polymer of monosaccharides. A polysaccharide may have any number of repeat units. A polysaccharide may be composed of the same type of monosaccharide (“homopolysaccharide”) or it may be composed of more than one type of monosaccharide (“heteropolysaccharide”). Polysaccharides may be naturally or non-naturally occurring. Exemplary polysaccharides include starch, glycogen, dextran, chitin, cellulose, hyaluronate, and alginate.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present invention contemplates any and all such combinations to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (I) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

Compounds of Formula (I) described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” is meant to include salts of the active compounds that are prepared with relatively nontoxic acids or bases, depending on the particular substituents found on the compounds described herein. When compounds used in the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the present disclosure contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

In addition to salt forms, the disclosure may employ compounds of Formula (I) in a prodrug form. Prodrugs are those compounds that readily undergo chemical changes under physiological conditions to provide the compounds useful in the present invention. Additionally, prodrugs can be converted to useful compounds of Formula (I) by chemical or biochemical methods in an ex vivo environment.

Certain compounds of Formula (I) described herein can exist in unsolvated forms as well as solvated forms, including hydrated forms. In general, the solvated forms are equivalent to unsolvated forms and are encompassed within the scope of the present invention. Certain compounds of Formula (I) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, dimethylsulfoxide (DMSO), tetrahydrofuran (THF), diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological effect of a compound of interest.

The symbol “ [symbol] ” as used herein refers to a connection to an entity, e.g., a polysaccharide polymer (e.g., hydrogel-forming polymer such as alginate) or an implantable element (e.g., a device or material). The connection represented by “ [symbol] ” may refer to direct attachment to the entity, e.g., a polymer or an implantable element, or may refer to linkage to the entity through an attachment group. An “attachment group,” as described herein, refers to a moiety for linkage of a compound of Formula (I) to an entity (e.g., a polymer or an implantable element as described herein), and may comprise any attachment chemistry known in the art. A listing of exemplary attachment groups is outlined in Bioconjugate Techniques (3^{rd} ed, Greg T. Hermanson, Waltham, MA: Elsevier, Inc, 2013). In some embodiments, an attachment group comprises alkyl, alkenyl, alkynyl, heteroalkyl, cycloalkyl, heterocyclyl, aryl, heteroaryl, —C(O)—, —OC(O)—, —N(R^{C})—, —N(R^{C})C(O)—, —C(O)N(R^{C})—, —N(R^{C})N(R^{D})—, —NCN—, —C(═N(R^{C})(R^{D}))O—, —S—, —S(O)ₓ, —OS(O)ₓ, —N(R^{C})S(O)ₓ, —S(O)ₓN(R^{C})—, —P(R^{F})_{y}—, —Si(OR^{A})₂—, —Si(R^{G})(OR^{A})—, —B(OR^{A})—, or a metal, wherein each of R^{A}, R^{C}, R^{D}, R^{F}, R^{G}, x and y is independently as described herein. In some embodiments, an attachment group comprises an amine, ketone, ester, amide, alkyl, alkenyl, alkynyl, or thiol. In some embodiments, an attachment group is a cross-linker. In some embodiments, the attachment group is —C(O)(C₁-C₆-alkylene)-, wherein alkylene is substituted with R¹, and R¹ is as described herein. In some embodiments, the attachment group is —C(O)(C₁-C₆-alkylene)-, wherein alkylene is substituted with 1-2 alkyl groups (e.g., 1-2 methyl groups). In some embodiments, the attachment group is —C(O)C(CH₃)₂—. In some embodiments, the attachment group is —C(O)(methylene)-, wherein alkylene is substituted with 1-2 alkyl groups (e.g., 1-2 methyl groups). In some embodiments, the attachment group is —C(O)CH(CH₃)—. In some embodiments, the attachment group is —C(O)C(CH₃)—.

### Modified Saccharide Monomers

The present invention features a polysaccharide polymer comprising a saccharide monomer, wherein the saccharide monomer comprises a hydroxyl-modifying agent covalently bound to the saccharide monomer. In some embodiments, the hydroxyl-modifying agent is covalently bound to the atom (e.g., the carbon atom) bearing the hydroxyl moiety or formerly bearing the hydroxyl moiety. In some embodiments, the hydroxyl-modifying agent is covalently bound to the hydroxyl moiety. In some embodiments, the hydroxyl-modifying agent comprises an alkyl, alkenyl, alkynyl, heteroalkyl, alkoxy, amine, amide, haloalkyl, haloalkoxy, ester, ether, carbamate, aryl, heteroaryl, cycloalkyl, or heterocyclyl moiety. In some embodiments, the hydroxyl-modifying agent comprises an amine.

The saccharide monomer may be any saccharide monomer, e.g., a naturally occurring saccharide monomer or a non-naturally occurring saccharide monomer. The saccharide monomer may comprise 1, 2, 3, 4, 5, 6, 7, 8, or more hydroxyl moieties. In an embodiment, the saccharide monomer is a triose, tetrose, pentose, hexose, heptose, or octose. In an embodiment, the saccharide monomer further comprises an additional functional group, e.g., a carboxylic acid or an amine. In an embodiment, the saccharide monomer further comprises a plurality of additional functional groups. In an embodiment, the saccharide monomer is selected from glucose, galactose, mannose, allose, altrose, talose, idose, gulose, fructose, ribose, arabinose, lyxose, xylose, rhamnose, glucuronic acid, galacturonic acid, mannuronic acid, and guluronic acid. In an embodiment, the saccharide monomer is mannuronic acid or guluronic acid. In an embodiment, the saccharide monomer is activated prior to modification. In an embodiment, the saccharide monomer undergoes a ring-opening reaction prior to modification with a hydroxyl modifying agent. In an embodiment, the saccharide monomer is oxidized (e.g., with periodate, IO⁴⁻) prior to hydroxyl group modification.

In an embodiment, the saccharide monomer has a structure of Formula (I):

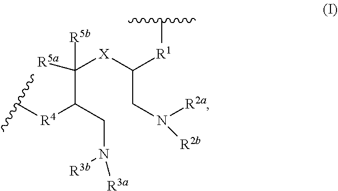

or a pharmaceutically acceptable salt thereof, wherein X is O, NR⁶, or S; each of R¹ and R⁴ is independently absent, alkylene, alkenylene, alkynylene, heteroalkylene, haloalkylene, —N(R^{7a})—, —O—, —C(O)—, —C(O)O—, —C(O)N(R^{C})—, or —N(R^{c})C(O)—, wherein alkylene, alkenylene, alkynylene, heteroalkylene, and haloalkylene is optionally substituted by one or more R⁸; R^{2a}, R^{2b}, R^{3a}, and R^{3b} are each independently hydrogen, alkyl, heteroalkyl, haloalkyl, C(O)R^{B}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted by one or more R⁹, wherein at least one of R^{2a} and R^{2b} and at least one of R^{3a} and R^{3b} is not hydrogen; each of R^{5a} and R^{5b} is independently hydrogen, alkyl, alkenyl, alkynyl, heteroalkyl, haloalkyl, N(R^{7a})(R^{7b}), OR^{A}, C(O)R^{B}, C(O)OR^{A}, C(O)N(R^{C})(R^{D}), N(R^{C})C(O)R^{B}, halogen, cyano, azido, aryl, heteroaryl, cycloalkyl, heterocyclyl, an afibrotic compound, or a peptide, wherein alkyl, alkenyl, alkynyl, heteroalkyl, haloalkyl, aryl, heteroaryl, cycloalkyl, and heterocyclyl is optionally substituted by one or more R¹; R⁶ is hydrogen, alkyl, heteroalkyl, or haloalkyl, wherein alkyl, heteroalkyl, and haloalkyl is optionally substituted by one or more R⁹; R^{7a} and R^{7b} are each independently hydrogen, alkyl, cycloalkyl, or heterocyclyl, wherein alkyl, cycloalkyl, or heterocyclyl is optionally substituted by one or more R⁹; each R⁸ is independently alkyl, alkenyl, alkynyl, heteroalkyl, haloalkyl, halogen, oxo, cyano, azido, aryl, heteroaryl, cycloalkyl, heterocyclyl, OR^{A}, N(R^{C})(R^{D}), C(O)OR^{A}, C(O)R^{B}, C(O)N(R^{C})(R^{D}), or N(R^{C})C(O)R^{B}, wherein each alkyl, alkenyl, alkynyl, heteroalkyl, haloalkyl, aryl, heteroaryl, cycloalkyl, and heterocyclyl is substituted by 0-12 R¹⁰; R^{A} is hydrogen, alkyl, alkenyl, alkynyl, heteroalkyl, haloalkyl, aryl, heteroaryl, cycloalkyl, heterocyclyl, an afibrotic compound, or a peptide, wherein each alkyl, alkenyl, alkynyl, heteroalkyl, haloalkyl, aryl, heteroaryl, cycloalkyl, and heterocyclyl is substituted by 0-12 R¹⁰; R^{B}, R^{C}, and R^{D} are alkyl, alkenyl, alkynyl, heteroalkyl, haloalkyl, halogen, aryl, heteroaryl, cycloalkyl, heterocyclyl, an afibrotic compound, or a peptide; wherein each alkyl, alkenyl, alkynyl, heteroalkyl, haloalkyl, aryl, heteroaryl, cycloalkyl, and is optionally substituted by one or more R¹⁰; or R^{B} and R^{C} are taken together with the atoms to which they are attached to form a 3-10 membered heterocyclyl or heteroaryl ring, each of which is optionally substituted with one or more R¹⁰; each R⁹ is independently alkyl, heteroalkyl, haloalkyl, halogen, oxo, OR^{A}, N(R^{C})(R^{D}), C(O)OR^{A}, C(O)R^{B}, C(O)N(R^{C})(R^{D}), or N(R^{C})C(O)R^{B}, wherein each alkyl, heteroalkyl, and haloalkyl is optionally substituted by one or more R¹⁰; and each R¹⁰ is independently C₁₋₆ alkyl, halogen, oxo, cycloalkyl, or heterocyclyl.

In an embodiment, the saccharide monomer has a structure of Formula (I-a):

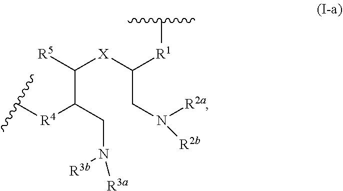

or a pharmaceutically acceptable salt thereof, wherein X is O, NR⁶, or S; R¹ is absent, alkylene, alkenylene, alkynylene, heteroalkylene, haloalkylene, —N(R^{7a})—, —O—, —C(O)—, —C(O)O—, —C(O)N(R^{C})—, or —N(R^{C})C(O)—, wherein alkylene, alkenylene, alkynylene, heteroalkylene, and haloalkylene is optionally substituted by one or more R⁸; R^{2a}, R^{2b}, R^{3a}, and R^{3b} are each independently hydrogen, alkyl, heteroalkyl, haloalkyl, C(O)R^{B}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted by one or more R⁹, wherein at least one of R^{2a} and R^{2b} and at least one of R^{3a} and R^{3b} is not hydrogen; each R⁴ is absent, alkylene, alkenylene, alkynylene, heteroalkylene, haloalkylene, —N(R^{7a})—, —O—, —C(O)—, —C(O)O—, —C(O)N(R^{C})—, or —N(R^{C})C(O)—, wherein each alkylene, alkenylene, alkynylene, heteroalkylene, and is optionally substituted by one or more R⁸; R⁵ is hydrogen, alkyl, alkenyl, alkynyl, heteroalkyl, haloalkyl, N(R^{7a})(R^{7b}), OR^{A}, C(O)R^{B}, C(O)OR^{A}, C(O)N(R^{C})(R^{D}), N(R^{C})C(O)R^{B}, halogen, cyano, azido, aryl, heteroaryl, cycloalkyl, heterocyclyl, an afibrotic compound, or a peptide, wherein alkyl, alkenyl, alkynyl, heteroalkyl, haloalkyl, aryl, heteroaryl, cycloalkyl, and heterocyclyl is optionally substituted by one or more R⁸; R⁶ is hydrogen, alkyl, heteroalkyl, or haloalkyl, wherein alkyl, heteroalkyl, and haloalkyl is optionally substituted by one or more R⁹, R^{7a} and R^{7b} are each independently hydrogen, alkyl, cycloalkyl, or heterocyclyl, wherein alkyl, cycloalkyl, or heterocyclyl is optionally substituted by one or more R⁹; each R⁸ is independently alkyl, alkenyl, alkynyl, heteroalkyl, haloalkyl, halogen, oxo, cyano, azido, aryl, heteroaryl, cycloalkyl, heterocyclyl, OR^{A}, N(R^{C})(R^{D}), C(O)OR^{A}, C(O)R^{B}, C(O)N(R^{C})(R^{D}), or N(R^{C})C(O)R^{B}, wherein each alkyl, alkenyl, alkynyl, heteroalkyl, haloalkyl, aryl, heteroaryl, cycloalkyl, and heterocyclyl is substituted by 0-12 R¹⁰; R^{A} is hydrogen, alkyl, alkenyl, alkynyl, heteroalkyl, haloalkyl, aryl, heteroaryl, cycloalkyl, heterocyclyl, an afibrotic compound, or a peptide, wherein each alkyl, alkenyl, alkynyl, heteroalkyl, haloalkyl, aryl, heteroaryl, cycloalkyl, and heterocyclyl is substituted by 0-12 R¹⁰; R^{B}, R^{C}, and R^{D} are alkyl, alkenyl, alkynyl, heteroalkyl, haloalkyl, halogen, aryl, heteroaryl, cycloalkyl, heterocyclyl, an afibrotic compound (e.g., a, or a peptide; wherein each alkyl, alkenyl, alkynyl, heteroalkyl, haloalkyl, aryl, heteroaryl, cycloalkyl, and is optionally substituted by one or more R¹⁰; or R^{B} and R^{C} are taken together with the atoms to which they are attached to form a 3-10 membered heterocyclyl or heteroaryl ring, each of which is optionally substituted with one or more R¹⁰; each R⁹ is independently alkyl, heteroalkyl, haloalkyl, halogen, oxo, OR^{A}, N(R^{C})(R^{D}), C(O)OR^{A}, C(O)R^{B}, C(O)N(R^{C})(R^{D}), or N(R^{C})C(O)R^{B}, wherein each alkyl, heteroalkyl, and haloalkyl is optionally substituted by one or more R¹⁰; and each R¹⁰ is independently alkyl, halogen, oxo, cycloalkyl, or heterocyclyl.

In an embodiment, the saccharide monomer has a structure of Formula (I-b):

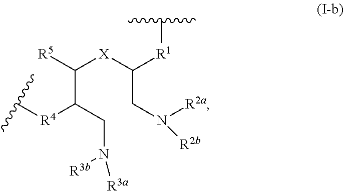

or a pharmaceutically acceptable salt thereof, wherein X is O, NR⁶, or S; R¹ is absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{c})C(O)—, wherein alkylene, alkenylene, alkynylene, heteroalkylene, and haloalkylene is optionally substituted by one or more R⁸; R^{2a}, R^{2b}, R^{3a}, and R^{3b} are each independently hydrogen, C₁₋₆ alkyl, C₁₋₆ heteroalkyl, C₁₋₆ haloalkyl, C(O)R^{B}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted by one or more R⁹, wherein at least one of R^{2a} and R^{2b} and at least one of R^{3a} and R^{3b} is not hydrogen; each R⁴ is absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{C})C(O)—, wherein each alkylene, alkenylene, alkynylene, heteroalkylene, and is optionally substituted by one or more R⁸; R⁵ is hydrogen, C₁₋₆ alkyl, C₂₋₆ alkenyl, C₂₋₆ alkynyl, C₁₋₆ heteroalkyl, C₁₋₆ haloalkyl, N(R^{7a})(R^{7b}), OR^{A}, C(O)R^{B}, C(O)OR^{A}, C(O)N(R^{C})(R^{D}), N(R^{C})C(O)R^{B}, halogen, cyano, azido, aryl, heteroaryl, cycloalkyl, heterocyclyl, an afibrotic compound, or a peptide, wherein alkyl, alkenyl, alkynyl, heteroalkyl, haloalkyl, aryl, heteroaryl, cycloalkyl, and heterocyclyl is optionally substituted by one or more R¹; R⁶ is hydrogen, C₁₋₆ alkyl, C₁₋₆ heteroalkyl, or C₁₋₆ haloalkyl, wherein alkyl, heteroalkyl, and haloalkyl is optionally substituted by one or more R⁹; R^{7a} and R^{7b} are each independently hydrogen, C₁₋₆ alkyl, cycloalkyl, or heterocyclyl, wherein alkyl, cycloalkyl, or heterocyclyl is optionally substituted by one or more R⁹; each R⁸ is independently C₁-C₆ alkyl, C₂-C₆ alkenyl, C₂-C₆ alkynyl, C₁-C₆ heteroalkyl, C₁-C₆ haloalkyl, halogen, oxo, cyano, azido, aryl, heteroaryl, cycloalkyl, heterocyclyl, OR^{A}N(R^{C})(R^{D}), C(O)OR^{A}, C(O)R^{B}, C(O)N(R^{C})(R^{D}), or N(R^{C})C(O)R^{B}, wherein each alkyl, alkenyl, alkynyl, heteroalkyl, haloalkyl, aryl, heteroaryl, cycloalkyl, and heterocyclyl is substituted by 0-12 R¹⁰; R^{A} is hydrogen, C₁-C₆ alkyl, C₂-C₆ alkenyl, C₂-C₆ alkynyl, C₁-C₆ heteroalkyl, C₁-C₆ haloalkyl, aryl, heteroaryl, cycloalkyl, heterocyclyl, an afibrotic compound, or a peptide, wherein each alkyl, alkenyl, alkynyl, heteroalkyl, haloalkyl, aryl, heteroaryl, cycloalkyl, and heterocyclyl is substituted by 0-12 R¹⁰; R^{B}, R^{C}, and R^{D} are C₁-C₆ alkyl, C₂-C₆ alkenyl, C₂-C₆ alkynyl, C₁-C₆ heteroalkyl, C₁-C₆ haloalkyl, halogen, aryl, heteroaryl, cycloalkyl, heterocyclyl, an afibrotic compound, or a peptide; wherein each alkyl, alkenyl, alkynyl, heteroalkyl, haloalkyl, aryl, heteroaryl, cycloalkyl, and is optionally substituted by one or more R¹⁰; or R^{B} and R^{C} are taken together with the atoms to which they are attached to form a 3-10 membered heterocyclyl or heteroaryl ring, each of which is optionally substituted with one or more R¹⁰; each R⁹ is independently C₁-C₆ alkyl, C₁-C₆ heteroalkyl, C₁-C₆ haloalkyl, halogen, oxo, OR^{A}, N(R^{C})(R^{D}), C(O)OR^{A}, C(O)R^{B}, C(O)N(R^{C})(R^{D}), or N(R^{C})C(O)R^{B}, wherein each alkyl, heteroalkyl, and haloalkyl is optionally substituted by one or more R¹⁰; and each R¹⁰ is independently C₁₋₆ alkyl, halogen, oxo, cycloalkyl, or heterocyclyl.

In an embodiment, X is O. In an embodiment, R¹ is OR^{A}. In an embodiment, R⁵ is C(O)OR^{A} or C(O)N(R^{C})(R^{D}). In an embodiment, R⁵ is C(O)OR^{A}. In an embodiment, R^{A} is hydrogen. In an embodiment, R⁵ is C(O)N(R^{C})(R^{D}). In an embodiment, R^{C} and R^{D} are each independently hydrogen, an afibrotic compound (e.g., an afibrotic compound provided in Table 2), or a peptide. In an embodiment, one of R^{C} and R^{D} is hydrogen and the other of R^{C} and R^{D} is independently an afibrotic compound (e.g., an afibrotic compound provided in Table 2) or a peptide.

In an embodiment, R² is C₁₋₆ alkyl, C₂₋₆ alkenyl, C₂₋₆ alkynyl, C₁₋₆ heteroalkyl, C₁₋₆ haloalkyl, C(O)R^{B}, aryl, heteroaryl, cycloalkyl, or heterocyclyl. In an embodiment, R³ is C₁₋₆ alkyl, C₂₋₆ alkenyl, C₂₋₆ alkynyl, C₁₋₆ heteroalkyl, C₁₋₆ haloalkyl, C(O)R^{B}, aryl, heteroaryl, cycloalkyl, or heterocyclyl. In an embodiment, one of R² and R³ is independently C₁₋₆ alkyl, C₁₋₆ heteroalkyl, C₁₋₆ haloalkyl, C(O)R^{B}, and the other of R² and R³ is independently hydrogen. In an embodiment, R⁴ is OR^{A}.

In an embodiment the afibrotic compound is selected from a compound of Table 2. In an embodiment, the afibrotic compound is a compound disclosed in one of U.S. patent Ser. No.: 11,266,606; US Patent Publication No.: US20200263196A1 or US Patent Publication No. US20200039943A1, each of which is incorporated herein by reference in its entirety.

In an embodiment, the saccharide monomer has a structure of Formula (I-c):

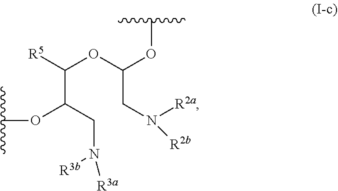

or a pharmaceutically acceptable salt thereof, wherein each of R^{2a}, R^{2b}, R^{3a}, R^{3b}, R⁵, and subvariables thereof are as defined in Formula (I-a).

In an embodiment, the saccharide monomer has a structure of Formula (I-d):

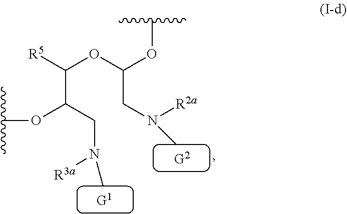

or a pharmaceutically acceptable salt thereof, wherein each of R^{2a}, R^{2b}, R^{3a}, R^{3b}, R⁵, and subvariables thereof are as defined in Formula (I-a), and each of G¹ and G² is independently hydrogen, an afibrotic compound, or a peptide.

In an embodiment, the saccharide monomer has a structure of Formula (I-e):

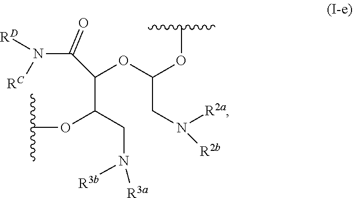

or a pharmaceutically acceptable salt thereof, wherein each of R^{2a}R^{2b}, R^{3a}, R^{3b}, R^{C}, and R^{D} and subvariables thereof are as defined in Formula (I-a).

In an embodiment, the saccharide monomer has a structure of Formula (I-f):

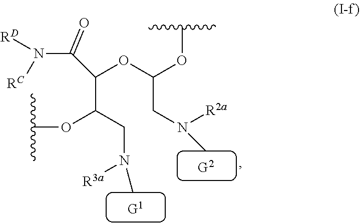

or a pharmaceutically acceptable salt thereof, wherein each of R^{2a}R^{3a}, R^{C}, and R^{D} and subvariables thereof are as defined in Formula (I-a), and each of G¹ and G² is independently hydrogen, an afibrotic compound (e.g., a compound of Table 2), or a peptide (e.g., a peptide of Table 3).

In an embodiment, of Formula (I-f), one of G¹ and G² is independently an afibrotic compound. In an embodiment, G¹ and G² are independently an afibrotic compound. In an embodiment, of Formula (I-f), G¹ and G² are independently a compound selected from Table 2. In an embodiment, of Formula (I-f), G¹ and G² are independently a compound selected from Table 2 and each of R^{2a}, R^{3a}, R^{C}, and R^{D} are hydrogen. In an embodiment, of Formula (I-f), G¹ and G² are independently a compound selected from Table 2; each of R^{2a} and R^{3a} are hydrogen; and each of R^{C} and R^{D} is independently hydrogen or a peptide selected from Table 3.

In an embodiment, the saccharide monomer has a structure of Formula (I-g):

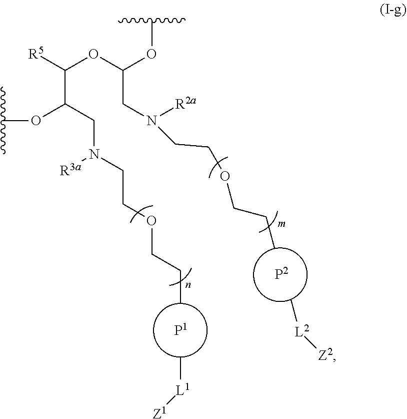

or a pharmaceutically acceptable salt thereof, wherein each of R^{2a}, R^{3a}, R⁵, R^{7a}, R⁸ and subvariables thereof are as definedin Formula (I-a); P¹ and P² are each independently aryl, heteroaryl, cycloalkyl, or heterocyclyl, each of which are optionally substituted by one or more R¹¹; L¹ and L² are each independently absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{c})C(O)—, wherein each alkylene, alkenylene, alkynylene, heteroalkylene, and is optionally substituted by one or more R⁸; Z¹ and Z² are each independently hydrogen, cycloalkyl, heterocyclyl, aryl, or heteroaryl, wherein each cycloalkyl, heterocyclyl, aryl, or heteroaryl is optionally substituted by one or more R⁸; each of m and n is independently 1, 2, 3, 4, 5, 6, 7, 8, 9, 10, 11, or 12; R¹¹ is alkyl, alkenyl, alkynyl, heteroalkyl, halo, or cyano.

In an embodiment, the saccharide monomer has a structure of Formula (I-h):

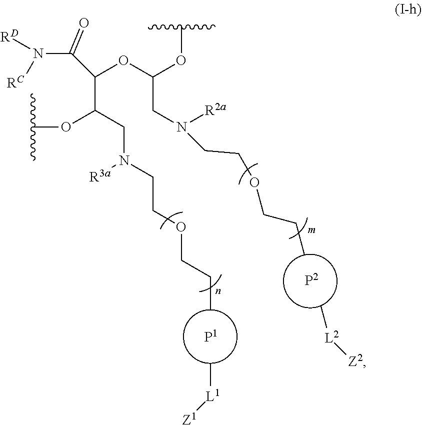

or a pharmaceutically acceptable salt thereof, wherein each of R^{2a}, R^{3a}, R^{C}, R^{D}, R^{7a}, R⁸, R¹¹, P¹, P², L¹, L², Z¹, Z² and subvariables thereof are as defined in Formulas (I-a) to (I-g); and each of m and n is independently 1, 2, 3, 4, 5, 6, 7, 8, 9, 10, 11, or 12.

In an embodiment, the saccharide monomer has a structure of Formula (I-i):

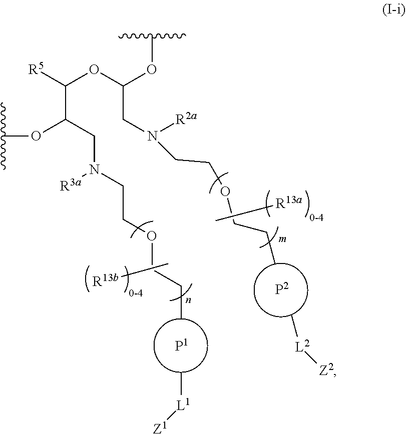

or a pharmaceutically acceptable salt thereof, wherein each of R^{2a}, R^{3a}, R⁵, P¹, P², L¹, L², Z¹, Z² and subvariables thereof are as defined in Formulas (I-a)-(I-h); R^{13a} and R^{13b} are each independently hydrogen, deuterium, C₁₋₆ alkyl, C₁₋₆ heteroalkyl, C₁₋₆ haloalkyl, or halo; and each of m and n is independently 1, 2, 3, 4, 5, 6, 7, 8, 9, 10, 11, or 12.

In an embodiment, the saccharide monomer has a structure of Formula (I-j):

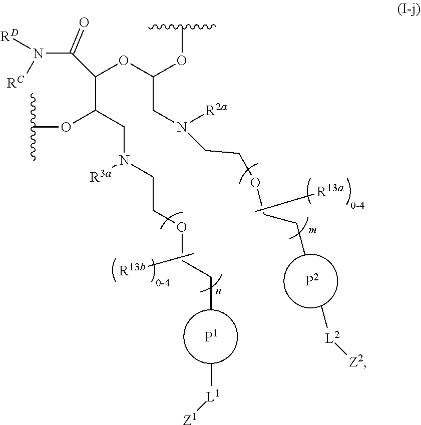

or a pharmaceutically acceptable salt thereof, wherein each of R^{2a}, R^{3a}, R^{C}, R^{D}, P¹, P², L¹, L², Z¹, Z², R^{13a} and R^{13b} and subvariables thereof are as defined in Formulas (I-a)-(I-i); each of m and n is independently 1, 2, 3, 4, 5, 6, 7, 8, 9, 10, 11, or 12.

In an embodiment, the saccharide monomer has a structure of Formula (I-k):

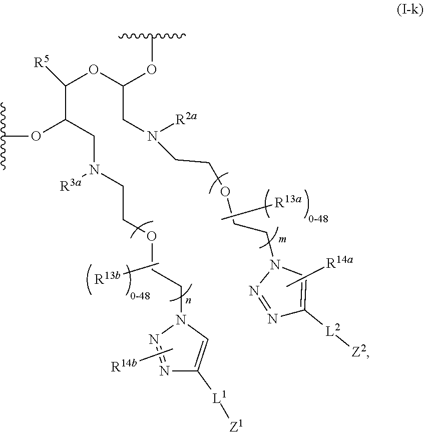

or a pharmaceutically acceptable salt thereof, wherein each of R^{2a}, R^{3a}, R⁵, L¹, L², Z¹, Z², R^{13a} and R^{13b} and subvariables thereof are as defined in Formulas (I-a)-(I-j); R^{14a} and R^{14b} are each independently hydrogen, C₁₋₆ alkylene, C₂₋₆ alkenylene, C₂₋₆ alkynylene, C₁₋₆ heteroalkylene, C₁₋₆ haloalkylene, halo, or cyano; and each of m and n is independently 1, 2, 3, 4, 5, 6, 7, 8, 9, 10, 11, or 12.

In an embodiment, the saccharide monomer has a structure of Formula (I-1):

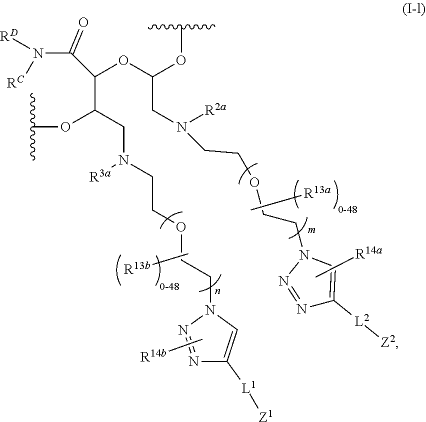

or a pharmaceutically acceptable salt thereof, wherein each of R^{2a}, R^{3a}, R^{C}, R^{D} L¹, L², Z¹, Z², R^{13a}, R^{13b}, R^{14a}, R^{14b} and subvariables thereof are as defined in Formulas (I-a)-(I-k); and each of m and n is independently 1, 2, 3, 4, 5, 6, 7, 8, 9, 10, 11, or 12.

In an embodiment, P¹ and P² are each independently heteroaryl. In an embodiment, P¹ and P² are each independently a monocyclic heteroaryl. In an embodiment, P¹ and P² are each independently a nitrogen-containing heteroaryl. In an embodiment, P¹ and P² are each independently a monocyclic, nitrogen-containing heteroaryl. In an embodiment, P¹ and P² are each independently a 5-membered heteroaryl. In some embodiments, P is a 5-membered nitrogen-containing heteroaryl.

In an embodiment, P¹ and P² are each triazolyl substituted by one or more R¹² In an embodiment, R¹² is deuterium, alkyl, alkenyl, alkynyl, heteroalkyl, halogen, cyano, azido, —N(R^{C1})(R^{D1}), —N(R^{C1})C(O)R^{B1}, —C(O)N(R^{C1}), —S(O)ₓR^{E1}, —N(R^{C1})S(O)ₓR^{E1}, —S(O)ₓN(R^{C1})(R^{D}1), —P(R^{F1})_{y} cycloalkyl, heterocyclyl, aryl, heteroaryl. In an embodiment, R¹² is deuterium, alkyl, heteroalkyl, halogen, cyano, or azido. In an embodiment, R¹² is chloro. In an embodiment, P¹ and P² are each independently

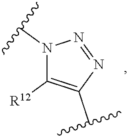

wherein R¹² is hydrogen, deuterium, C₁₋₆ alkyl, C₁₋₆ heteroalkyl, C₁₋₆ haloalkyl, or halo.

In an embodiment, the saccharide monomer has a structure of Formula (I-m):

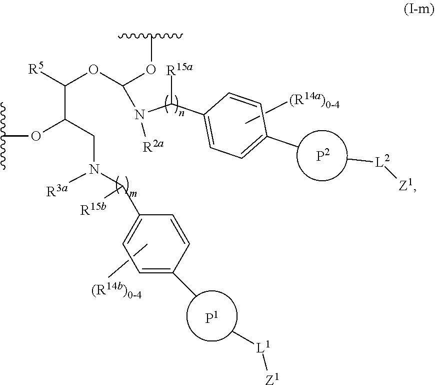

or a pharmaceutically acceptable salt thereof, wherein each of R^{2a}, R^{3a}, R⁵, P¹, P², L¹, L², Z¹, Z², R^{14a}, R^{14b} and subvariables thereof are as defined in Formulas (I-a)-(I-l); R^{15a} and R^{15b} are each independently hydrogen, deuterium, alkyl, heteroalkyl, haloalkyl, or halo; and each of m and n is independently 1, 2, 3, 4, or 5.

In an embodiment, the saccharide monomer has a structure of Formula (I-n):

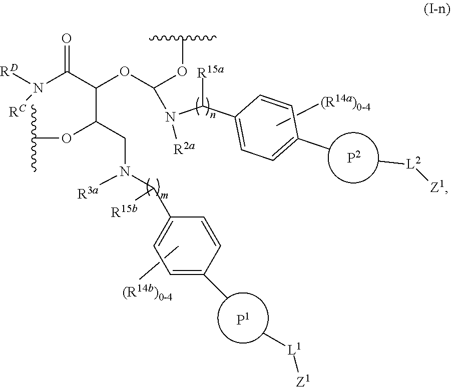

or a pharmaceutically acceptable salt thereof, wherein each of R^{2a}, R^{3a}, R⁵, P¹, P², L¹, L², Z¹, Z², R^{14a}, R^{14b}, R^{15a}, R^{15b} and subvariables thereof are as defined in Formulas (I-a)-(I-m); and each of m and n is independently 1, 2, 3, 4, or 5.

In an embodiment, P¹ and P² are each independently

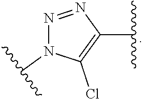 [SMILES: *c1nnn(*)c1Cl]

In an embodiment, P¹ and P² are each independently

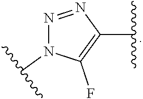 [SMILES: *c1nnn(*)c1F]

In an embodiment, P¹ and P² are each independently

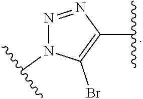 [SMILES: *c1nnn(*)c1Br]

In an embodiment, P¹ and P² are each independently

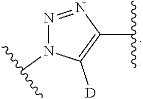 [SMILES: *c1nnn(*)c1[2H]]

In an embodiment, P¹ and P² are each independently

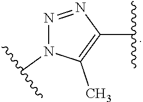 [SMILES: *c1nnn(*)c1C]

In an embodiment, P¹ and P² are each independently

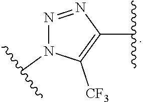 [SMILES: *c1nnn(*)c1C(F)(F)F]

In an embodiment, P¹ and P² are each independently

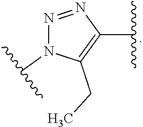 [SMILES: *c1nnn(*)c1CC]

In an embodiment, P¹ and P² are each independently

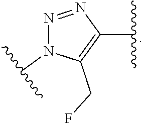 [SMILES: *c1nnn(*)c1CF]

In an embodiment, P¹ and P² are each independently

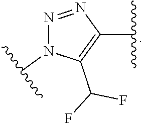 [SMILES: *c1nnn(*)c1C(F)F]

In an embodiment, L¹ and L² are each independently absent or C₁₋₆ alkylene (e.g., —CH₂—).

In an embodiment, Z¹ and Z² are each independently aryl, heteroaryl, or heterocyclyl. In an embodiment, Z¹ and Z² are each independently heterocyclyl. In an embodiment, Z¹ and Z² are each independently monocyclic or bicyclic heterocyclyl. In an embodiment, Z¹ and Z² are each independently a 4-membered heterocyclyl, 5-membered heterocyclyl, or 6-membered heterocyclyl. In an embodiment, Z¹ and Z² are each independently a 4-membered heterocyclyl. In an embodiment, Z¹ and Z² are each independently a 6-membered heterocyclyl. In an embodiment, Z¹ and Z² are each independently a nitrogen-containing heterocyclyl. In an embodiment, Z¹ and Z² are each independently a sulfur-containing heterocyclyl. In an embodiment, Z¹ and Z² are each independently a 4-membered nitrogen-containing heterocyclyl. In an embodiment, Z¹ and Z² are each independently a 6-membered nitrogen-containing heterocyclyl. In an embodiment, Z¹ and Z² are each independently a 6-membered sulfur-containing heterocyclyl. In an embodiment, Z¹ and Z² are each independently a 6-membered heterocyclyl containing a nitrogen atom and a sulfur atom. In an embodiment, Z¹ and Z² are each independently a 4-membered nitrogen heterocyclyl optionally substituted with one R⁸ (e.g., —S(O)ₓR^{E1}). In an embodiment, R⁸ is —S(O)₂CH₃.

In an embodiment, Z¹ and Z² are each independently 3-(methylsulfonyl)azetidinyl. In an embodiment, Z¹ and Z² are each independently

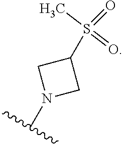 [SMILES: *N1CC(S(C)(=O)=O)C1]

In an embodiment, Z¹ and Z² are each independently thiomorpholinyl-1,1-dioxidyl. In an embodiment, Z¹ and Z² are each independently

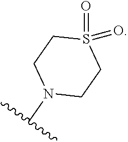 [SMILES: *N1CCS(=O)(=O)CC1]

In an embodiment, the saccharide moiety is selected from a compound in Table 1, or a pharmaceutically acceptable salt thereof

TABLE 1
Exemplary compounds of Formula (I)

| Compound No. | Structure |
|---|---|
| 100 | 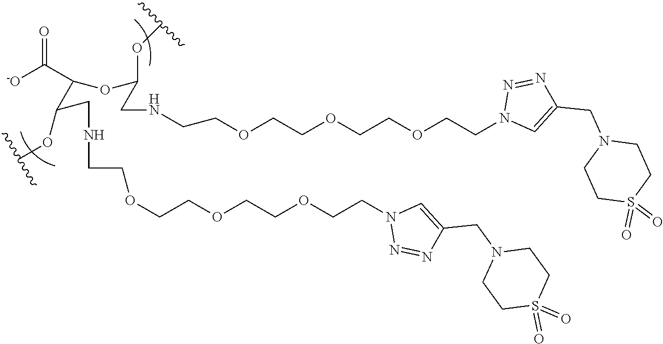 |
| 101 | 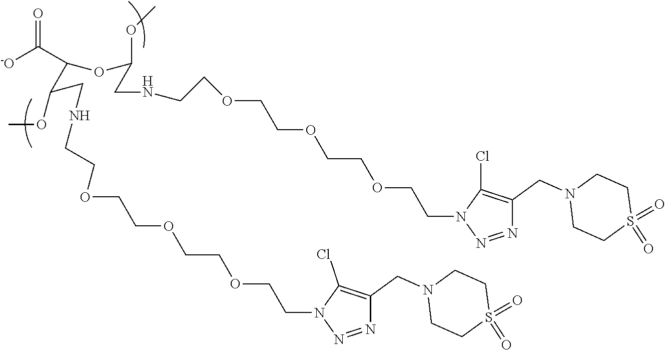 |
| 102 | 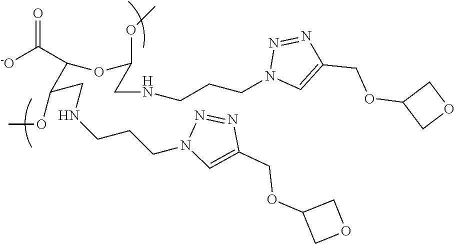 |
| 103 | 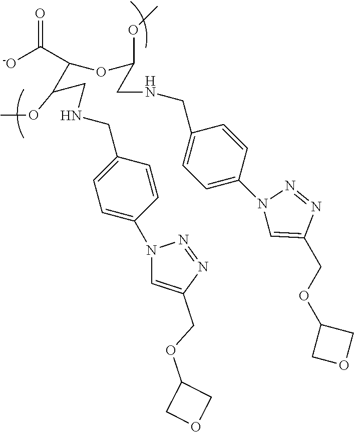 |
| 104 | 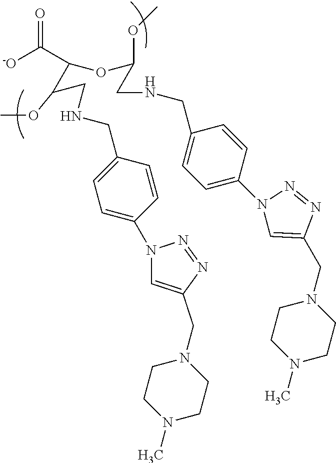 |
| 105 | 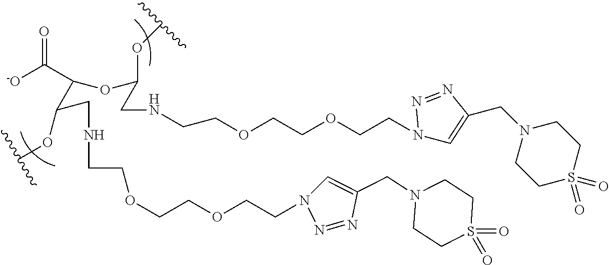 |
| 106 | 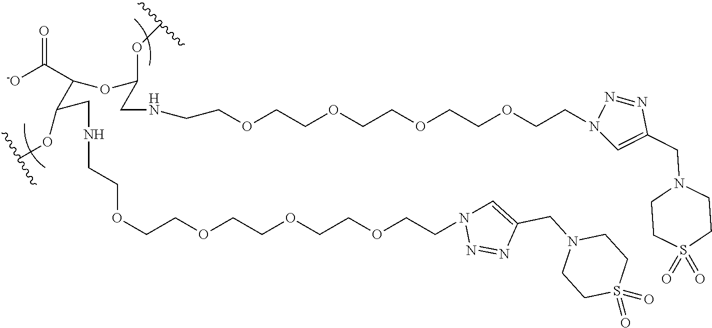 |
| 107 | 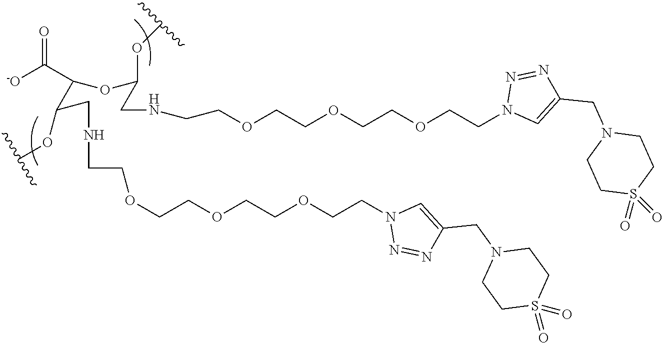 |
| 108 | 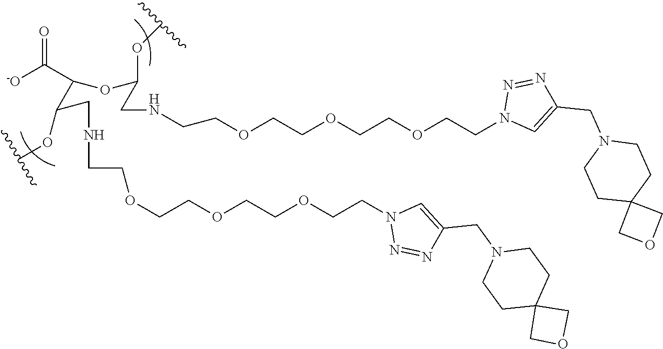 |
| 109 | 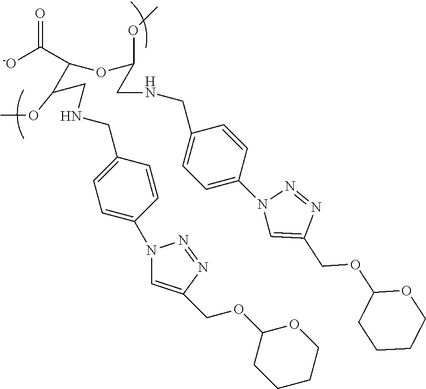 |
| 110 | 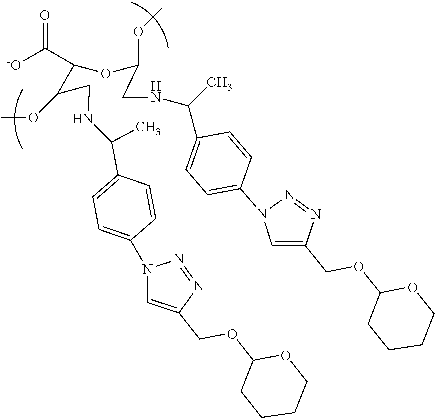 |
| 111 | 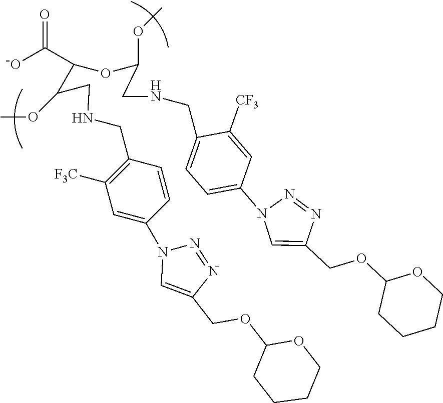 |
| 112 | 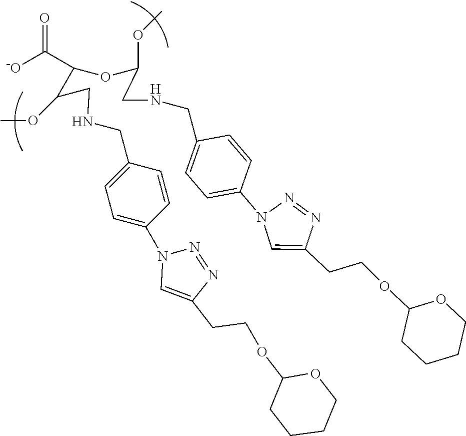 |
| 113 | 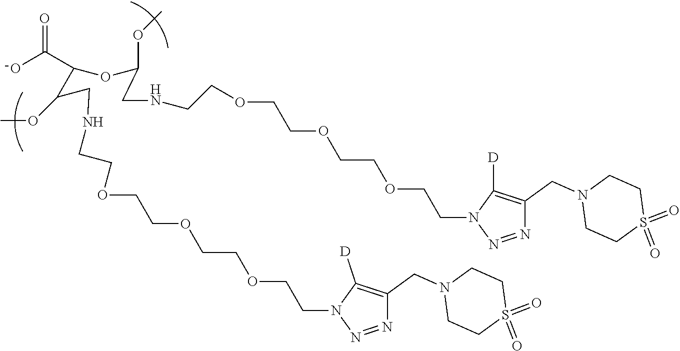 |
| 114 | 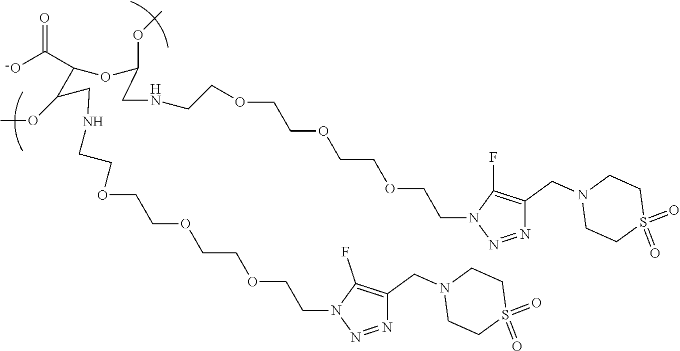 |
| 115 | 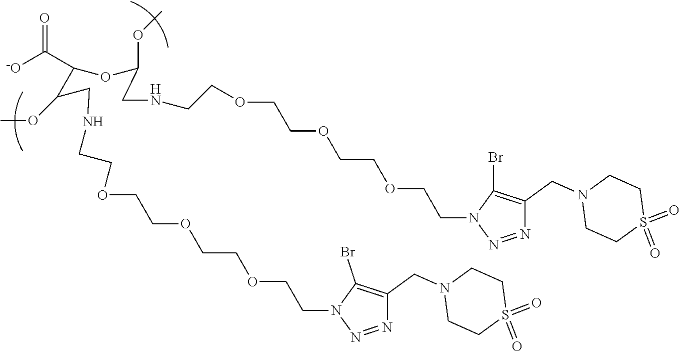 |
| 116 | 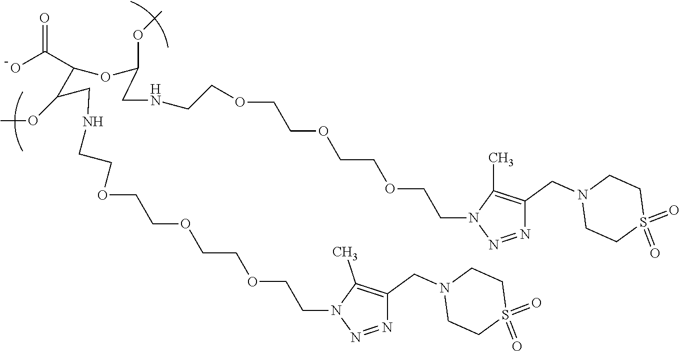 |
| 117 | 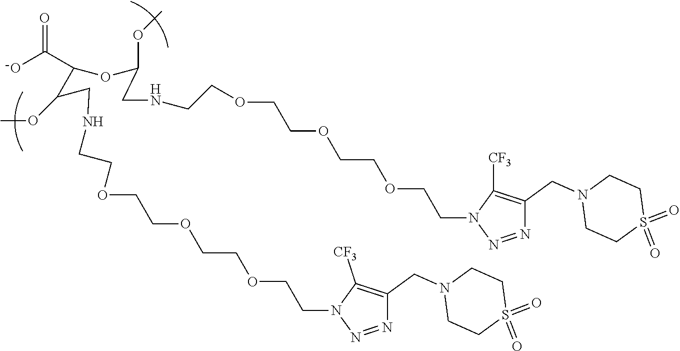 |
| 118 | 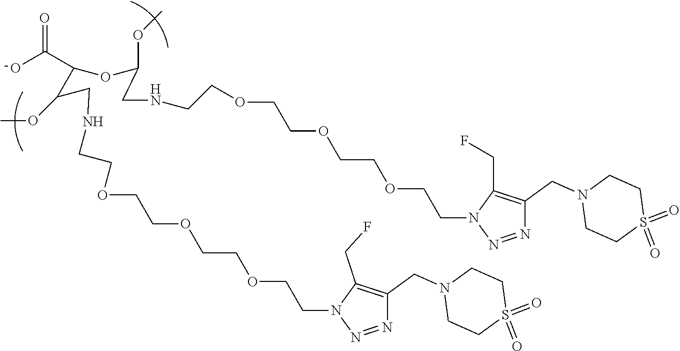 |
| 119 | 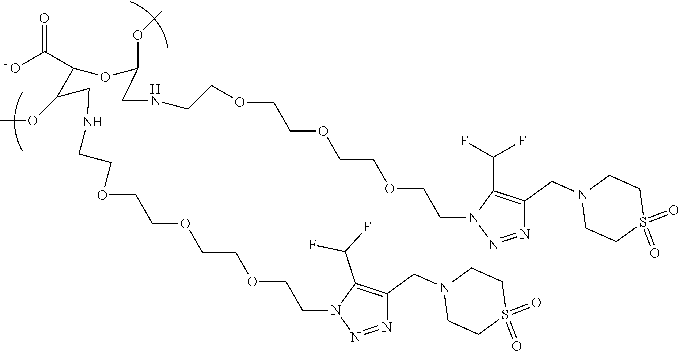 |
| 120 | 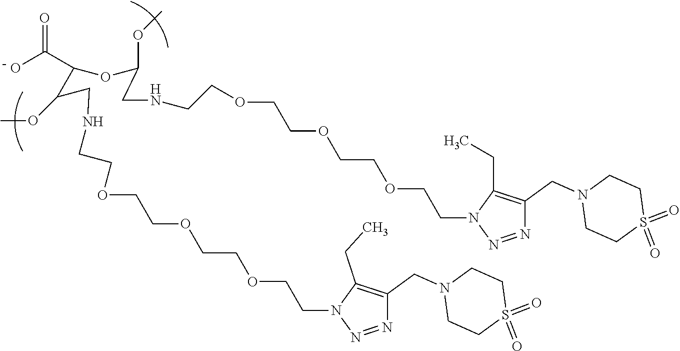 |
| 121 | 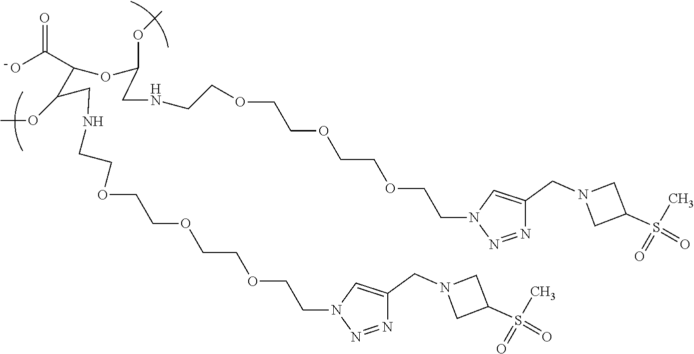 |
| 122 | 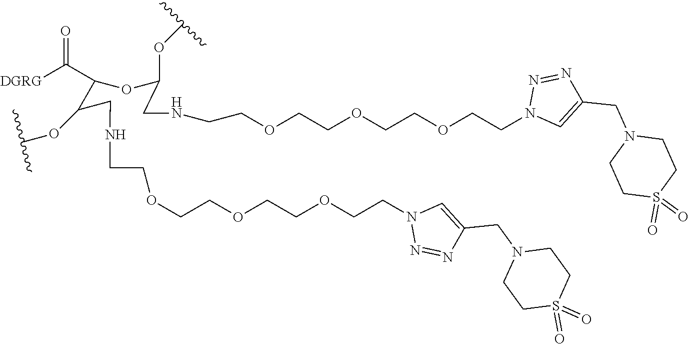 |
| 123 | 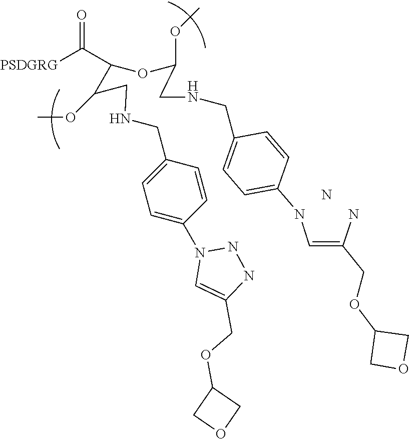 |
| 124 | 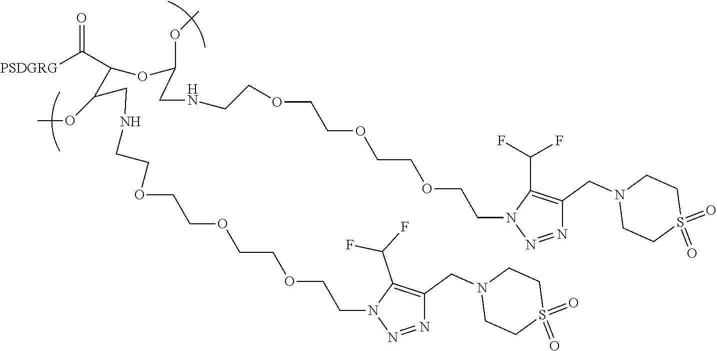 |
| 125 | 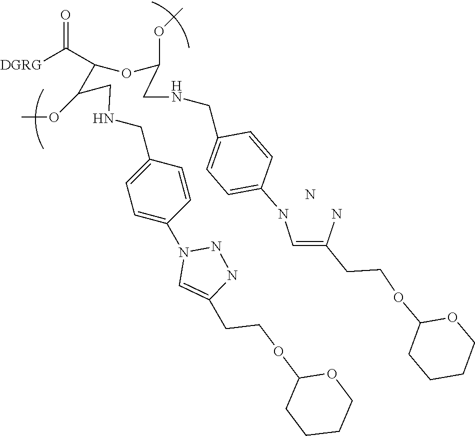 |
| 126 | 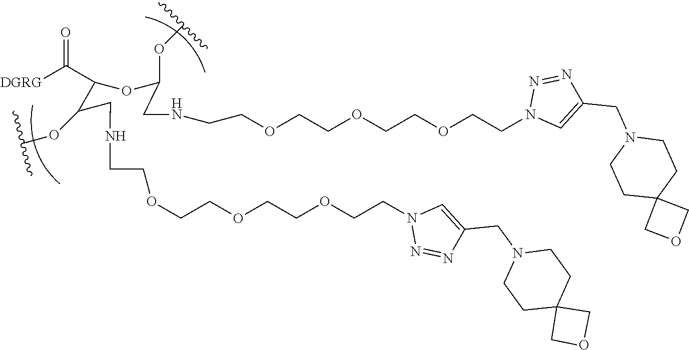 |
| 127 | 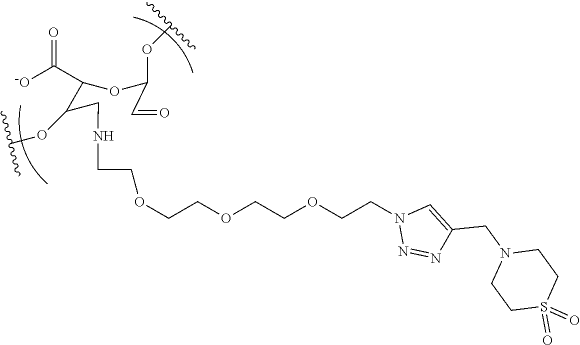 |
| 128 | 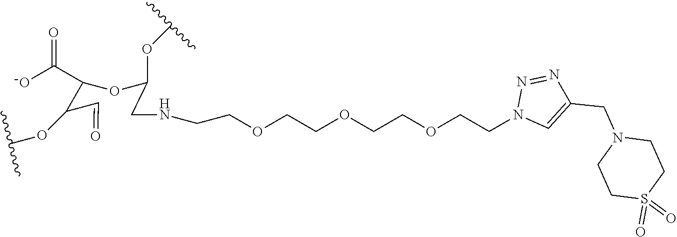 [SMILES: *OC(CNCCOCCOCCOCCn1cc(CN2CCS(=O)(=O)CC2)nn1)OC(C(=O)O)C(C=O)O*] |
| 129 | 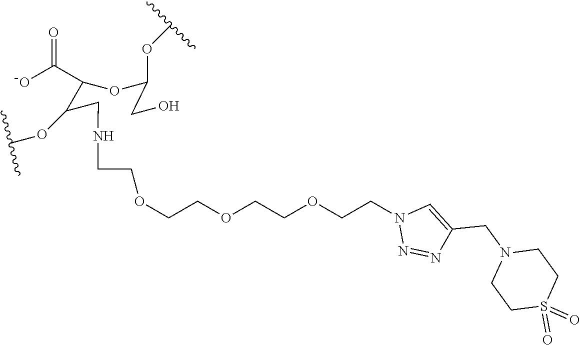 [SMILES: *OC(CO)OC(C(=O)O)C(CNCCOCCOCCOCCn1cc(CN2CCS(=O)(=O)CC2)nn1)O*] |
| 130 | 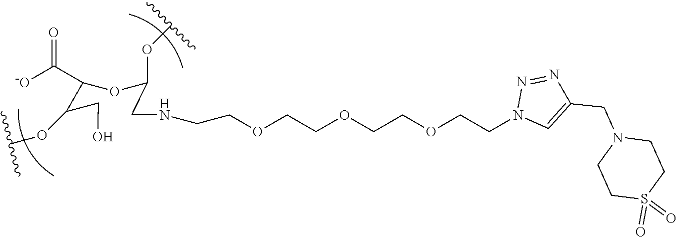 |

In an embodiment, the saccharide moiety is selected from Compounds 100-130. In an embodiment, the saccharide moiety is selected from Compound 100, 101, and 102. In an embodiment, the saccharide moiety is Compound 100. In an embodiment, the saccharide moiety is Compound 101. In an embodiment, the saccharide moiety is Compound 102. In an embodiment, the saccharide moiety is Compound 103. In an embodiment, the saccharide moiety is Compound 104. In an embodiment, the saccharide moiety is Compound 105. In an embodiment, the saccharide moiety is Compound 106. In an embodiment, the saccharide moiety is Compound 107. In an embodiment, the saccharide moiety is Compound 108. In an embodiment, the saccharide moiety is Compound 109. In an embodiment, the saccharide moiety is Compound 110. In an embodiment, the saccharide moiety is Compound 111. In an embodiment, the saccharide moiety is Compound 112. In an embodiment, the saccharide moiety is Compound 113. In an embodiment, the saccharide moiety is Compound 114. In an embodiment, the saccharide moiety is Compound 115. In an embodiment, the saccharide moiety is Compound 116. In an embodiment, the saccharide moiety is Compound 117. In an embodiment, the saccharide moiety is Compound 118. In an embodiment, the saccharide moiety is Compound 119. In an embodiment, the saccharide moiety is Compound 120. In an embodiment, the saccharide moiety is Compound 121. In an embodiment, the saccharide moiety is Compound 122. In an embodiment, the saccharide moiety is Compound 123. In an embodiment, the saccharide moiety is Compound 124. In an embodiment, the saccharide moiety is Compound 125. In an embodiment, the saccharide moiety is Compound 126. In an embodiment, the saccharide moiety is Compound 127. In an embodiment, the saccharide moiety is Compound 128. In an embodiment, the saccharide moiety is Compound 129. In an embodiment, the saccharide moiety is Compound 130.

In an embodiment, the saccharide moiety further comprises an afibrotic compound, e.g., covalently bound to another functional group on the saccharide moiety (e.g., the saccharide moiety of Formula (I)). In an embodiment, the afibrotic compound is selected from a compound shown in Table 2.

Exemplary compounds of Formula (I)

| Compound No. | Structure |
|---|---|
| 200 | 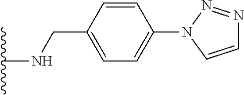 [SMILES: *NCc1ccc(-n2ccnn2)cc1] |
| 201 | 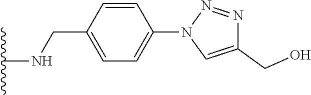 [SMILES: *NCc1ccc(-n2cc(CO)nn2)cc1] |
| 202 | 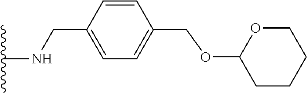 [SMILES: *NCc1ccc(COC2CCCCO2)cc1] |
| 203 | 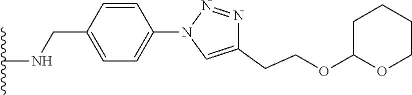 [SMILES: *NCc1ccc(-n2cc(CCOC3CCCCO3)nn2)cc1] |
| 204 | 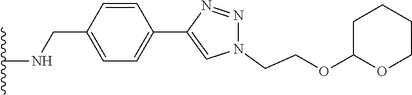 [SMILES: *NCc1ccc(-c2cn(CCOC3CCCCO3)nn2)cc1] |
| 205 | 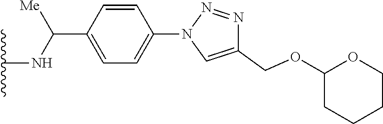 [SMILES: *NC(C)c1ccc(-n2cc(COC3CCCCO3)nn2)cc1] |
| 206 | 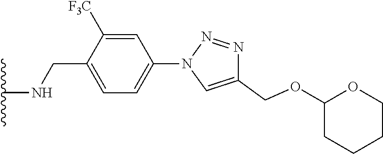 [SMILES: *NCc1ccc(-n2cc(COC3CCCCO3)nn2)cc1C(F)(F)F] |
| 207 | 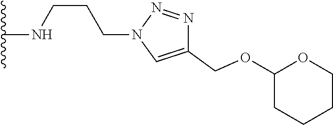 [SMILES: *NCCCn1cc(COC2CCCCO2)nn1] |
| 208 | 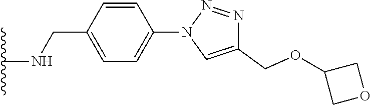 [SMILES: *NCc1ccc(-n2cc(COC3COC3)nn2)cc1] |
| 209 | 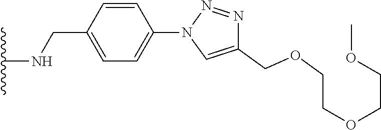 [SMILES: *NCc1ccc(-n2cc(COCCOCCOC)nn2)cc1] |
| 210 | 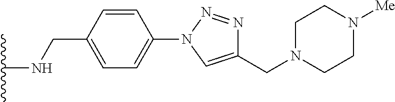 [SMILES: *NCc1ccc(-n2cc(CN3CCN(C)CC3)nn2)cc1] |
| 211 | 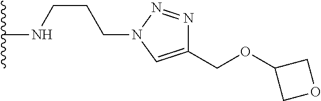 [SMILES: *NCCCn1cc(COC2COC2)nn1] |
| 212 | 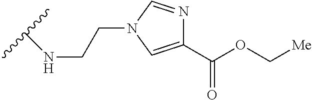 [SMILES: *NCCn1cnc(C(=O)OCC)c1] |
| 213 | 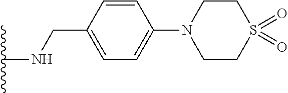 [SMILES: *NCc1ccc(N2CCS(=O)(=O)CC2)cc1] |
| 214 | 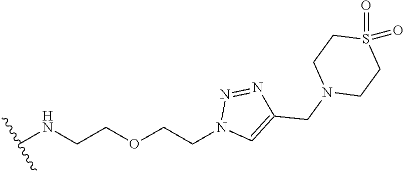 [SMILES: *NCCOCCn1cc(CN2CCS(=O)(=O)CC2)nn1] |
| 215 | 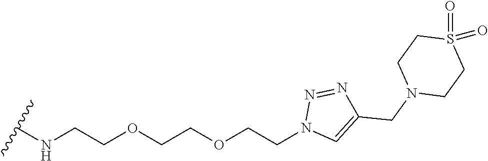 [SMILES: *NCCOCCOCCn1cc(CN2CCS(=O)(=O)CC2)nn1] |
| 216 | 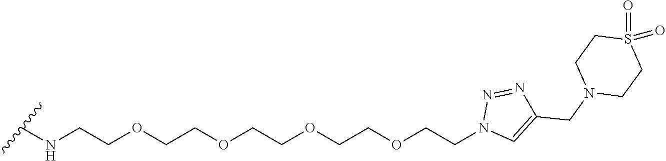 [SMILES: *NCCOCCOCCOCCOCCn1cc(CN2CCS(=O)(=O)CC2)nn1] |
| 217 | 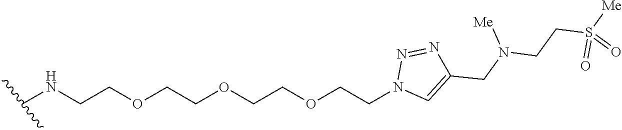 [SMILES: *NCCOCCOCCOCCn1cc(CN(C)CCS(C)(=O)=O)nn1] |
| 218 | 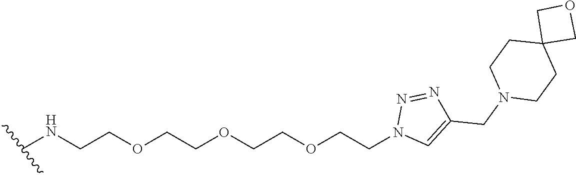 [SMILES: *NCCOCCOCCOCCn1cc(CN2CCC3(CC2)COC3)nn1] |
| 219 | 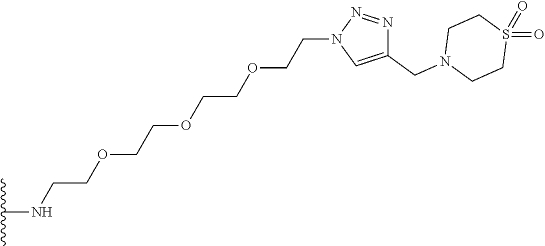 [SMILES: *NCCOCCOCCOCCn1cc(CN2CCS(=O)(=O)CC2)nn1] |
| 220 | 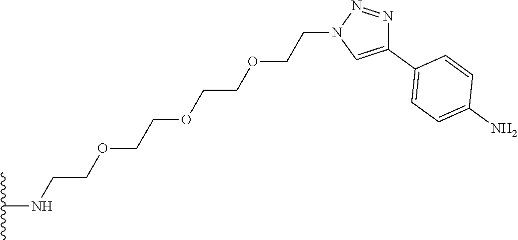 [SMILES: *NCCOCCOCCOCCn1cc(-c2ccc(N)cc2)nn1] |
| 221 | 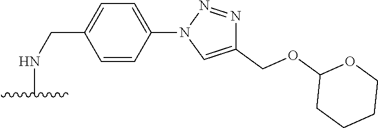 [SMILES: *NCc1ccc(-n2cc(COC3CCCCO3)nn2)cc1] |
| 222 | 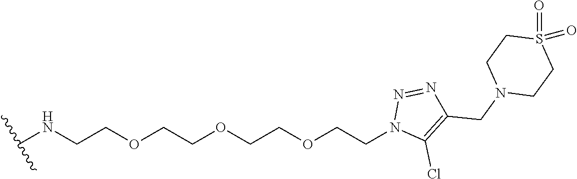 [SMILES: *NCCOCCOCCOCCn1nnc(CN2CCS(=O)(=O)CC2)c1Cl] |
| 223 | 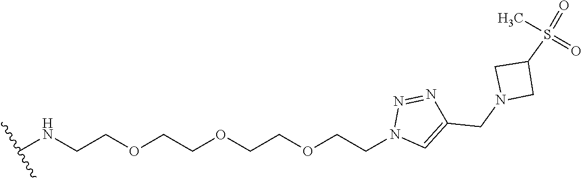 [SMILES: *NCCOCCOCCOCCn1cc(CN2CC(S(C)(=O)=O)C2)nn1] |
| 225 | 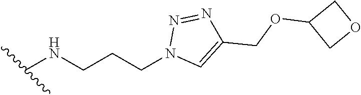 [SMILES: *NCCCn1cc(COC2COC2)nn1] |
| 226 | 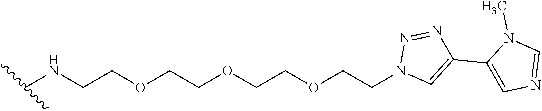 [SMILES: *NCCOCCOCCOCCn1cc(-c2cncn2C)nn1] |
| 227 | 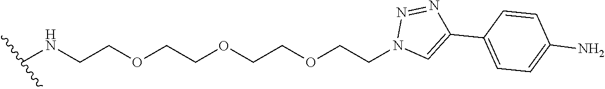 [SMILES: *NCCOCCOCCOCCn1cc(-c2ccc(N)cc2)nn1] |
| 228 | 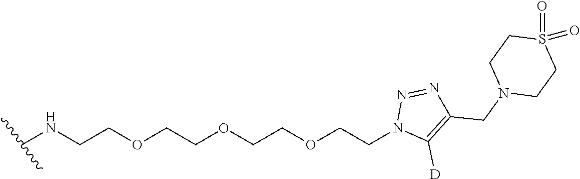 [SMILES: *NCCOCCOCCOCCn1nnc(CN2CCS(=O)(=O)CC2)c1[2H]] |
| 229 | 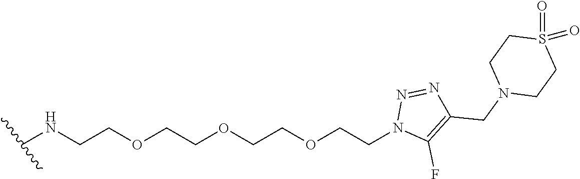 [SMILES: *NCCOCCOCCOCCn1nnc(CN2CCS(=O)(=O)CC2)c1F] |
| 230 | 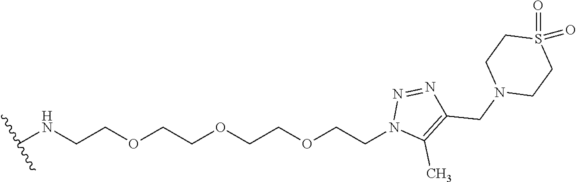 [SMILES: *NCCOCCOCCOCCn1nnc(CN2CCS(=O)(=O)CC2)c1C] |
| 231 | 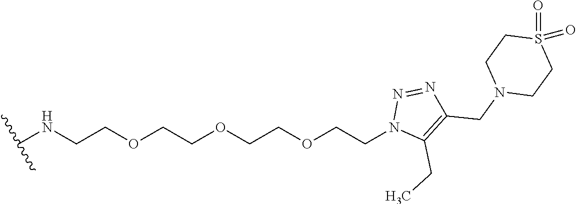 [SMILES: *NCCOCCOCCOCCn1nnc(CN2CCS(=O)(=O)CC2)c1CC] |
| 232 | 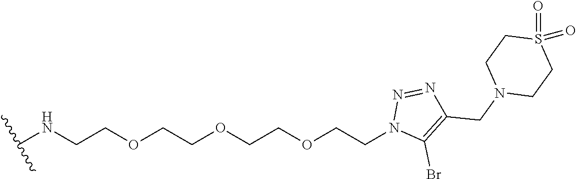 [SMILES: *NCCOCCOCCOCCn1nnc(CN2CCS(=O)(=O)CC2)c1Br] |
| 233 | 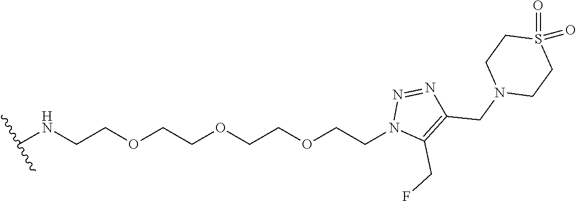 [SMILES: *NCCOCCOCCOCCn1nnc(CN2CCS(=O)(=O)CC2)c1CF] |
| 234 | 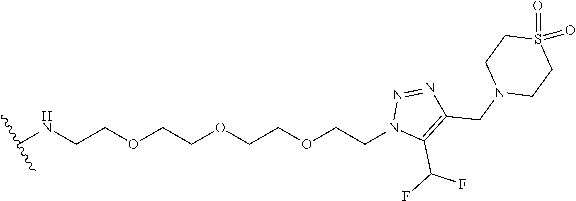 [SMILES: *NCCOCCOCCOCCn1nnc(CN2CCS(=O)(=O)CC2)c1C(F)F] |
| 235 | 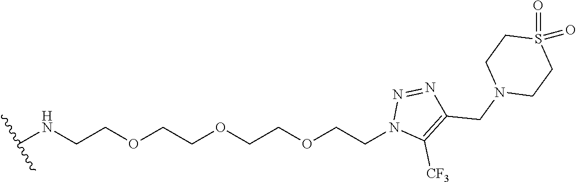 [SMILES: *NCCOCCOCCOCCn1nnc(CN2CCS(=O)(=O)CC2)c1C(F)(F)F] |
| 236 | 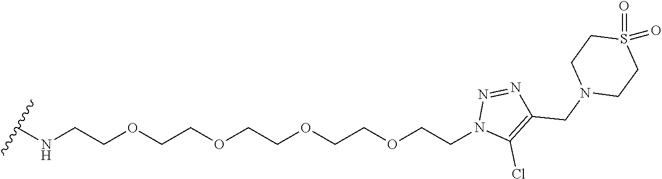 [SMILES: *NCCOCCOCCOCCOCCn1nnc(CN2CCS(=O)(=O)CC2)c1Cl] |
| 237 | 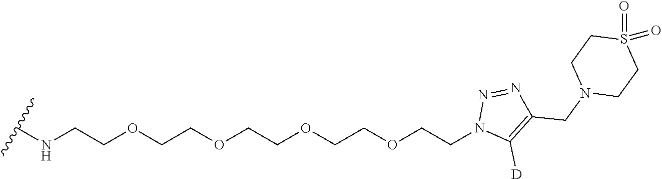 [SMILES: *NCCOCCOCCOCCOCCn1nnc(CN2CCS(=O)(=O)CC2)c1[2H]] |
| 238 | 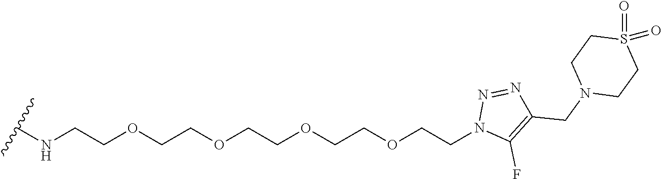 [SMILES: *NCCOCCOCCOCCOCCn1nnc(CN2CCS(=O)(=O)CC2)c1F] |
| 239 | 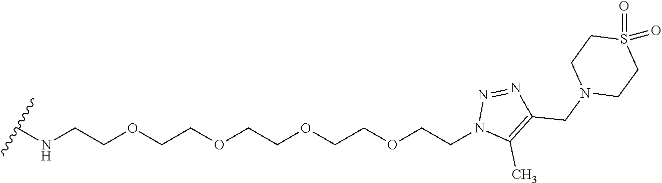 [SMILES: *NCCOCCOCCOCCOCCn1nnc(CN2CCS(=O)(=O)CC2)c1C] |
| 240 | 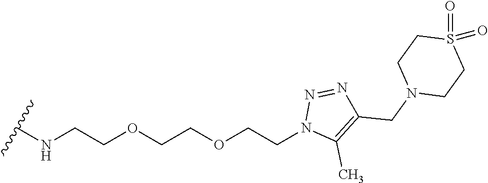 [SMILES: *NCCOCCOCCn1nnc(CN2CCS(=O)(=O)CC2)c1C] |
| 241 | 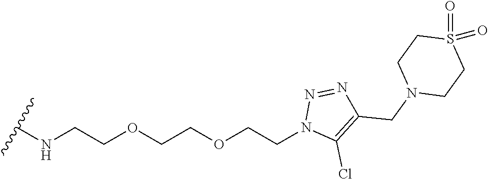 [SMILES: *NCCOCCOCCn1nnc(CN2CCS(=O)(=O)CC2)c1Cl] |
| 242 | 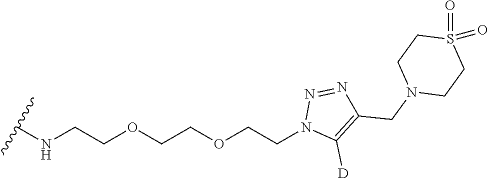 [SMILES: *NCCOCCOCCn1nnc(CN2CCS(=O)(=O)CC2)c1[2H]] |
| 243 | 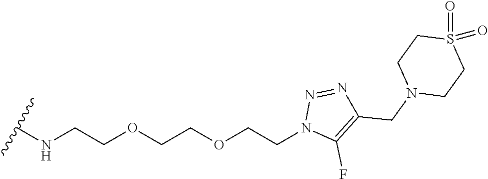 [SMILES: *NCCOCCOCCn1nnc(CN2CCS(=O)(=O)CC2)c1F] |
| 244 | 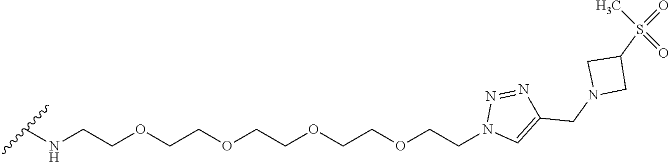 [SMILES: *NCCOCCOCCOCCOCCn1cc(CN2CC(S(C)(=O)=O)C2)nn1] |
| 245 | 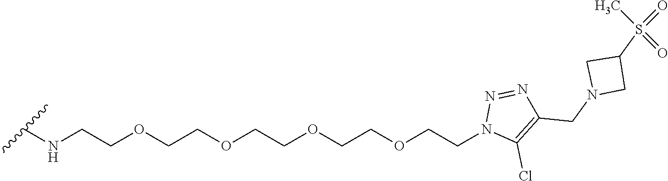 [SMILES: *NCCOCCOCCOCCOCCn1nnc(CN2CC(S(C)(=O)=O)C2)c1Cl] |
| 246 | 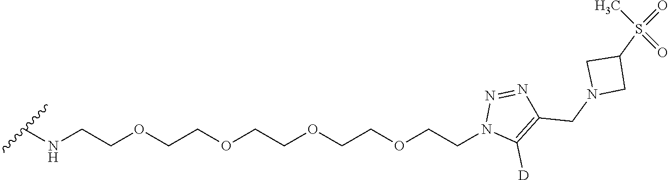 [SMILES: *NCCOCCOCCOCCOCCn1nnc(CN2CC(S(C)(=O)=O)C2)c1[2H]] |
| 247 | 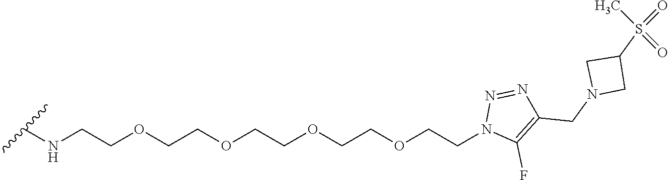 [SMILES: *NCCOCCOCCOCCOCCn1nnc(CN2CC(S(C)(=O)=O)C2)c1F] |
| 248 | 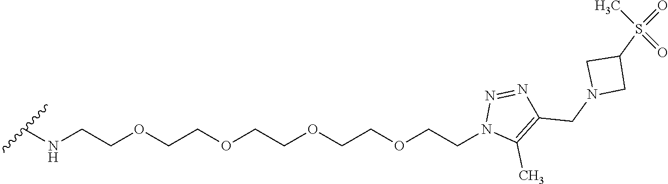 [SMILES: *NCCOCCOCCOCCOCCn1nnc(CN2CC(S(C)(=O)=O)C2)c1C] |
| 249 | 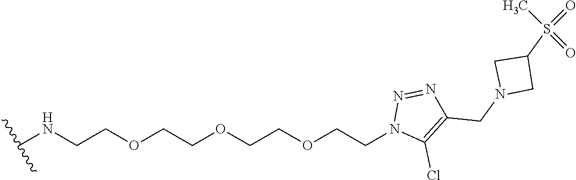 [SMILES: *NCCOCCOCCOCCn1nnc(CN2CC(S(C)(=O)=O)C2)c1Cl] |
| 250 | 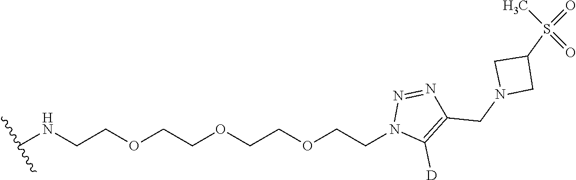 [SMILES: *NCCOCCOCCOCCn1nnc(CN2CC(S(C)(=O)=O)C2)c1[2H]] |
| 251 | 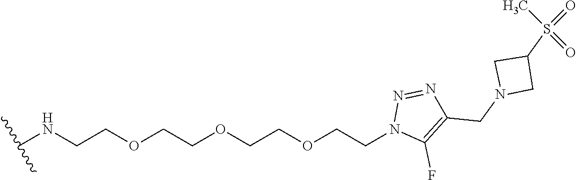 [SMILES: *NCCOCCOCCOCCn1nnc(CN2CC(S(C)(=O)=O)C2)c1F] |
| 252 | 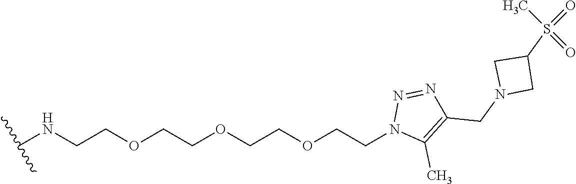 [SMILES: *NCCOCCOCCOCCn1nnc(CN2CC(S(C)(=O)=O)C2)c1C] |
| 253 | 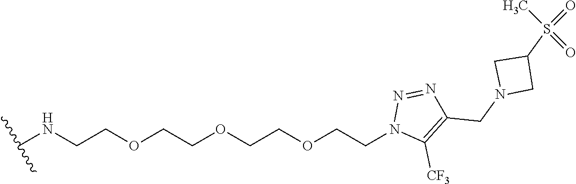 [SMILES: *NCCOCCOCCOCCn1nnc(CN2CC(S(C)(=O)=O)C2)c1C(F)(F)F] |
| 254 | 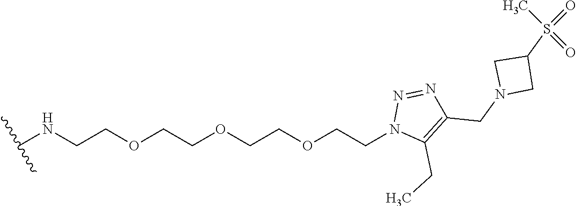 [SMILES: *NCCOCCOCCOCCn1nnc(CN2CC(S(C)(=O)=O)C2)c1CC] |

In an embodiment, the afibrotic compound is selected from Compound 200-254, or a salt thereof. In an embodiment, the afibrotic compound is Compound 200. In an embodiment, the afibrotic compound is Compound 201. In an embodiment, the afibrotic compound is Compound 202. In an embodiment, the afibrotic compound is Compound 203. In an embodiment, the afibrotic compound is Compound 204. In an embodiment, the afibrotic compound is Compound 205. In an embodiment, the afibrotic compound is Compound 206. In an embodiment, the afibrotic compound is Compound 207. In an embodiment, the afibrotic compound is Compound 208. In an embodiment, the afibrotic compound is Compound 209. In an embodiment, the afibrotic compound is Compound 210. In an embodiment, the afibrotic compound is Compound 211. In an embodiment, the afibrotic compound is Compound 212. In an embodiment, the afibrotic compound is Compound 213. In an embodiment, the afibrotic compound is Compound 214. In an embodiment, the afibrotic compound is Compound 215. In an embodiment, the afibrotic compound is Compound 216. In an embodiment, the afibrotic compound is Compound 200. In an embodiment, the afibrotic compound is Compound 217. In an embodiment, the afibrotic compound is Compound 218. In an embodiment, the afibrotic compound is Compound 219. In an embodiment, the afibrotic compound is Compound 220. In an embodiment, the afibrotic compound is Compound 221. In an embodiment, the afibrotic compound is Compound 222. In an embodiment, the afibrotic compound is Compound 223. In an embodiment, the afibrotic compound is Compound 224. In an embodiment, the afibrotic compound is Compound 225. In an embodiment, the afibrotic compound is Compound 226. In an embodiment, the afibrotic compound is Compound 227. In an embodiment, the afibrotic compound is Compound 228. In an embodiment, the afibrotic compound is Compound 229. In an embodiment, the afibrotic compound is Compound 230. In an embodiment, the afibrotic compound is Compound 231. In an embodiment, the afibrotic compound is Compound 232. In an embodiment, the afibrotic compound is Compound 233. In an embodiment, the afibrotic compound is Compound 234. In an embodiment, the afibrotic compound is Compound 235. In an embodiment, the afibrotic compound is Compound 236. In an embodiment, the afibrotic compound is Compound 237. In an embodiment, the afibrotic compound is Compound 238. In an embodiment, the afibrotic compound is Compound 239. In an embodiment, the afibrotic compound is Compound 240. In an embodiment, the afibrotic compound is Compound 241. In an embodiment, the afibrotic compound is Compound 242. In an embodiment, the afibrotic compound is Compound 243. In an embodiment, the afibrotic compound is Compound 244. In an embodiment, the afibrotic compound is Compound 245. In an embodiment, the afibrotic compound is Compound 246. In an embodiment, the afibrotic compound is Compound 247. In an embodiment, the afibrotic compound is Compound 248. In an embodiment, the afibrotic compound is Compound 249. In an embodiment, the afibrotic compound is Compound 250. In an embodiment, the afibrotic compound is Compound 251. In an embodiment, the afibrotic compound is Compound 252. In an embodiment, the afibrotic compound is Compound 253. In an embodiment, the afibrotic compound is Compound 254.

In an embodiment, the saccharide moiety further comprises a peptide, e.g., covalently bound to another functional group on the saccharide moiety (e.g., the saccharide moiety of Formula (I)). In an embodiment, the peptide is a cell-binding peptide. “Cell-binding peptide (CBP)”, as used herein, means a linear or cyclic peptide that comprises an amino acid sequence that is derived from the cell binding domain of a ligand for a cell-adhesion molecule (CAM) (e.g., that mediates cell-matrix junctions or cell-cell junctions). The CBP is less than 50, 40 30, 25, 20, 15 or 10 amino acids in length. In an embodiment, the CBP is between 3 and 12 amino acids in length, 4 and 10 amino acids in length, or is 3, 4, 5, 6, 7 8, 9 or 10 amino acids in length. The CBP amino acid sequence may be identical to the naturally occurring binding domain sequence or may be a conservatively substituted variant thereof. In an embodiment, the CAM ligand is a mammalian protein. In an embodiment, the CAM ligand is a human protein selected from the group of proteins listed in Table 1 below. In an embodiment, the CBP comprises a cell binding sequence listed in Table 1 below or a conservatively substituted variant thereof. In an embodiment, the CBP comprises at least one of the cell binding sequences listed in Table 3 below. In an embodiment, the CBP consists essentially of a cell binding sequence listed in Table 3 below. In an embodiment, the CBP is an RGD peptide, which means the peptide comprises the amino acid sequence RGD (SEQ TD NO: 20) and optionally comprises one or more additional amino acids located at one or both of the N-terminus and C-terminus. In an embodiment, the peptide is a peptide shown in Table 3. In this table, exemplary cell-binding proteins are recited, along with relevant cell-binding sequences.

TABLE 3
Exemplary Peptides

| Protein | Cell Binding Sequence |
|---|---|
| E-cadherin | SWELYYPLRANL (SEQ ID NO: 1) |
| N-cadherin | HAVDI (SEQ ID NO: 2) |
| Collagen I | DGEA (SEQ ID NO: 3) |
| Collagen IV | FYFDLR (SEQ ID NO: 4) |
| | GFOGER (SEQ ID NO: 5) |
| | P(GPP)₅GFOGER(GPP)₅ (SEQ ID NO: 6) |
| | (O in SEQ ID NO: 6 and 54 is 4-hydroxyproline) |
| Elastin | VAPG (SEQ ID NO: 7) |
| Fibrinogen | RGD (SEQ ID NO: 8) |
| | GPR (SEQ ID NO: 9) |
| Fibronectin | RGD (SEQ ID NO: 10) |
| | KQAGDV (SEQ ID NO: 11) |
| | PHSRN (SEQ ID NO: 12) |
| | PHSRNGGGGGGRGDS (SEQ ID NO: 13) |
| | REDV (SEQ ID NO: 14) |
| Laminin | IKVAV (SEQ ID NO: 15) |
| | SRARKQAASIKVAVADR (SEQ ID NO: 16) |
| | LRE (SEQ ID NO: 17) |
| | KQLREQ (SEQ ID NO: 18) |
| | YIGSR (SEQ ID NO: 19) |
| Nidogen-1 | RGD (SEQ ID NO: 20) |
| Osteopontin | SVVYGLR (SEQ ID NO: 21) |
| Tenascin C (TN-C) | AEIDGIEL (SEQ ID NO: 22) |
| Tenascin-R | RGD (SEQ ID NO: 23) |
| Tenascin-X | RGD (SEQ ID NO: 24) |
| Thrombospondin | VTCG (SEQ ID NO: 25) |
| | SVTCG (SEQ ID NO: 26) |
| Vitronectin | RGD (SEQ ID NO: 27) |
| Von Willebrand | RGD (SEQ ID NO: 28) |
| Factor | |

In an embodiment, the peptide comprises SEQ ID NO: 1. In an embodiment, the peptide comprises SEQ ID NO: 2. In an embodiment, the peptide comprises SEQ ID NO: 3. In an embodiment, the peptide comprises SEQ ID NO: 4. In an embodiment, the peptide comprises SEQ ID NO: 5. In an embodiment, the peptide comprises SEQ ID NO: 6. In an embodiment, the peptide comprises SEQ ID NO: 7. In an embodiment, the peptide comprises SEQ ID NO: 8. In an embodiment, the peptide comprises SEQ ID NO: 9. In an embodiment, the peptide comprises SEQ ID NO: 10. In an embodiment, the peptide comprises SEQ ID NO: 11. In an embodiment, the peptide comprises SEQ ID NO: 12. In an embodiment, the peptide comprises SEQ ID NO: 13. In an embodiment, the peptide comprises SEQ ID NO: 14. In an embodiment, the peptide comprises SEQ ID NO: 15. In an embodiment, the peptide comprises SEQ ID NO: 16. In an embodiment, the peptide comprises SEQ ID NO: 17. In an embodiment, the peptide comprises SEQ ID NO: 18. In an embodiment, the peptide comprises SEQ ID NO: 19. In an embodiment, the peptide comprises SEQ ID NO: 20. In an embodiment, the peptide comprises SEQ ID NO: 21. In an embodiment, the peptide comprises SEQ ID NO: 22. In an embodiment, the peptide comprises SEQ ID NO: 23. In an embodiment, the peptide comprises SEQ ID NO: 24. In an embodiment, the peptide comprises SEQ ID NO: 25. In an embodiment, the peptide comprises SEQ ID NO: 26. In an embodiment, the peptide comprises SEQ ID NO: 27. In an embodiment, the peptide comprises SEQ ID NO: 28.

In an embodiment, the peptide is a peptide disclosed in WO2020069429A1, which is incorporated herein by reference in its entirety.

### Modified Polymers

The polysaccharide polymers described herein comprise a saccharide moiety modified with a hydroxyl-modifying agent. In an embodiment, the polysaccharide polymer may be linear, branched, or cross-linked polysaccharide polymer, or a polysaccharide polymer of selected molecular weight ranges, degree of polymerization, viscosity or melt flow rate. Branched polysaccharide polymer can include one or more of the following types: star polymers, comb polymers, brush polymers, dendronized polymers, graft-co(polymers), ladders, and dendrimers. A polysaccharide polymer may be a thermoresponsive polymer, e.g., a gel (e.g., becomes a solid or liquid upon exposure to heat or a certain temperature) or a photocrosslinkable polymer. In some embodiments, a polysaccharide polymer is made up of a single type of repeating monomeric unit. In other embodiments, a polysaccharide polymer is made up of different types of repeating monomeric units (e.g., two types of repeating monomeric units, three types of repeating monomeric units, e.g., a polymeric blend).

In an embodiment, the polysaccharide polymer is a cellulose, e.g., carboxymethyl cellulose. In an embodiment, the polysaccharide polymer is a polylactide, a polyglycoside or a polycaprolactone. In an embodiment, the polysaccharide polymer is a hyaluronate, e.g., sodium hyaluronate. In an embodiment, the polymer is a collagen, elastin or gelatin.

In some embodiments, the polysaccharide polymer is a hydrogel-forming polymer. Hydrogel-forming polymers comprise a hydrophilic structure that renders them capable of holding large amounts of water in a three-dimensional network. Hydrogel-forming polymers may include polymers which form homopolymeric hydrogels, copolymeric hydrogels, or multipolymer interpenetrating polymeric hydrogels, and may be amorphous, semicrystalline, or crystalline in nature, e.g., as described in Ahmed (2015) J Adv Res 6:105-121. Exemplary hydrogel-forming polymers include proteins (e.g., collagen), gelatin, polysaccharides (e.g., starch, alginate, hyaluronate, agarose), and synthetic polysaccharides.

Exemplary polysaccharides include alginate, agar, agarose, carrageenan, hyaluronate, amylopectin, glycogen, gelatin, cellulose, amylose, chitin, chitosan, or a derivative or variant thereof, e.g., as described in Laurienzo (2010), Mar Drugs 9:2435-65. A polysaccharide polymer may comprise heparin, chondoitin sulfate, dermatan, dextran, or carboxymethylcellulose. In some embodiments, a polysaccharide polymer is a cross-linked polymer. In some embodiments, a polysaccharide polymer is a cell-surface polysaccharide.

In some embodiments, the polysaccharide polymer is an alginate. Algnate is a polysaccharide made up of β-D-mannuronic acid (M) and α-L-guluronic acid (G). In some embodiments, the alginate is a high guluronic acid (G) alginate, and comprises greater than about 50%, 55%, 60%, 65%, 70%, 75%, 80%, 85%, 90%, or more guluronic acid (G). In some embodiments, the alginate is a high mannuronic acid (M) alginate, and comprises greater than about 50%, 55%, 60%, 65%, 70%, 75%, 80%, 85%, 90%, or more mannuronic acid (M). In some embodiments, the ratio of M:G is about 1. In some embodiments, the ratio of M:G is less than 1. In some embodiments, the ratio of M:G is greater than 1. In some embodiments, the alginate has an approximate molecular weight of <75 kDa, and optionally a G:M ratio of ≥1.5. In some embodiments, the alginate has an approximate molecular weight of 75 kDa to 150 kDa and optionally a G:M ratio of ≥1.5. In some embodiments, the alginate has an approximate molecular weight of 150 to 250 kDa and optionally a G:M ratio of ≥1.5.

A polysaccharide polymer (e.g., any of the polymers described herein, for example, any of the alginates described herein) comprising a saccharide moiety having the structure of Formula (I) or a pharmaceutically acceptable salt thereof may be modified on one or more monomeric units. In some embodiments, at least 0.5 percent of the saccharide monomers of a polysaccharide polymer have the structure of Formula (I) (e.g., at least 1, 2.5, 5, 10, 15, 20, 25, 30, 35, 40, 45, 50, 55, 60, 65, 70, 75, 80, 85, 90, 95, 99 percent, or more of the saccharide monomers have the structure of Formula (I). In some embodiments, 0.5 to 50%, 10 to 90%, 10 to 50%, or 25-75%, of the saccharide monomers of a polysaccharide polymer have the structure of Formula (I). In some embodiments, 1 to 20% of the saccharide monomers of a polysaccharide polymer have the structure of Formula (I). In some embodiments, 1 to 10% of the saccharide monomers of a polysaccharide polymer have the structure of Formula (I).

In some embodiments, the polysaccharide polymer (when comprising a saccharide monomer having the structure of Formula I) comprises an increase in % N (as compared with unmodified polymer) of at least 0.1, 0.2, 0.5, 1.0, 1.5, 2, 2.5, 3, 3.5, 4, 4.5, 5, 5.5, 6, 6.5, 7, 7.5, 8, 8.5, 9, 9.5, or 10% N by weight, where % N is determined by elemental analysis and corresponds to the amount of compound of Formula (I) in the modified polymer.

In some embodiments, the polysaccharide polymer (when comprising a saccharide monomer having the structure of Formula ((I)) comprises an increase in % N (as compared with unmodified polymer) of 0.1 to 10% N by weight, where % N is determined by elemental analysis and corresponds to the amount of compound of Formula (I) in the modified polymer.

In some embodiments, the polysaccharide polymer (when comprising a saccharide monomer having the structure of Formula (I)) comprises an increase in % N (as compared with unmodified polymer) of 0.1 to 2% N by weight, where % N is determined by elemental analysis and corresponds to the amount of compound of Formula (I) in the modified polymer.

In some embodiments, the polysaccharide polymer (when comprising a saccharide monomer having the structure of Formula (I)) comprises an increase in % N (as compared with unmodified polymer) of 2 to 4% N by weight, where % N is determined by elemental analysis and corresponds to the amount of compound of Formula (I) in the modified polymer.

In some embodiments, the polysaccharide polymer (when comprising a saccharide monomer having the structure of Formula (I)) comprises an increase in % N (as compared with unmodified polymer) of 4 to 8% N by weight, where % N is determined by elemental analysis and corresponds to the amount of compound of Formula (I) in the modified polymer.

In some embodiments, any of the polysaccharide polymers described herein (e.g., an alginate) comprise a saccharide monomer having one or more of Formula (I-a), (I-b), (I-c), (I-d), (I-e), (I-f), or a pharmaceutically acceptable salt thereof. In some embodiments, the polymer (e.g., an alginate) is modified with a compound shown in Table 2. In some embodiments, a polymer (e.g., an alginate) modified with a compound of Formula (I) is not a modified polymer described in any one of WO2012/112982, WO2012/167223, WO2014/153126, WO2016/187225, WO2016/019391, WO2017/075630, WO 2017/075631, WO 2018/067615, WO 2019/169333, and US 2016-0030359.

### Implantable Elements

The disclosure also features an implantable element (e.g., a device or material) comprising a polysaccharide polymer of Formula (I) or a pharmaceutically acceptable salt thereof, as described herein. The surface of the implantable element may further comprise a material modified with an afibrotic compound, e.g., as shown in Table 2. In an embodiment, the polysaccharide polymer of Formula (I) present on a surface (e.g., an exterior surface or interior surface) of the implantable element. The implantable element comprising a polysaccharide polymer of Formula (I) may have an improved property compared to a reference implantable element, e.g., an otherwise identical implantable element that lacks polysaccharide polymer of Formula (I). In an embodiment, the improved property is a reduced foreign body response to the implantable element when administered to a subject (e.g., lower amount and/or later occurrence of PFO).

In some embodiments, the implantable element comprises a cell. In some embodiments, the cell is an engineered cell. In some embodiments, the cell is entirely or partially disposed with the implantable element. The implantable element may comprise an enclosing element that encapsulates or coats a cell, in part or in whole. In an embodiment, an implantable element comprises an enclosing component that is formed, or could be formed, in situ on or surrounding a cell, e.g., a plurality of cells, e.g., a cluster of cells, or on a microcarrier, e.g., a bead, or a matrix comprising a cell or cells.

Implantable elements can include any material, such as a polymer or other material described herein. In some embodiments, an implantable element is made up of one material or many types of materials. Implantable elements can comprise non-organic or metal components or materials, e.g., steel (e.g., stainless steel), titanium, other metal or alloy. Implantable elements can include nonmetal components or materials, e.g., ceramic, or hydroxyapatite elements.

Implantable elements can include components or materials that are made of a conductive material (e.g., gold, platinum, palladium, titanium, copper, aluminum, silver, metals, any combinations of these, etc.).

Implantable elements can include more than one component, e.g., more than one component disclosed herein, e.g., more than one of a metal, plastic, ceramic, composite, or hybrid material.

Exemplary implantable elements comprise materials such as metals, metallic alloys, ceramics, polymers, fibers, inert materials, and combinations thereof. An implantable element may be completely made up of one type of material, or may just refer to a surface or the surface of an implantable element (e.g., the outer surface or an inner surface).

In some embodiments, the implantable element (e.g., a device or material) comprises a metal or a metallic alloy. Exemplary metallic or metallic alloys include comprising titanium and titanium group alloys (e.g., nitinol, nickel titanium alloys, thermo-memory alloy materials), platinum, platinum group alloys, stainless steel, tantalum, palladium, zirconium, niobium, molybdenum, nickel-chrome, chromium molybdenum alloys, or certain cobalt alloys (e.g., cobalt-chromium and cobalt-chromium-nickel alloys. For example, a metallic material may be stainless steel grade 316 (SS 316L) (comprised of Fe, <0.3% C, 16-18.5% Cr, 10-14% Ni, 2-3% Mo, <2% Mn, <1% Si, <0.45% P, and <0.03% S). In metal-containing implantable elements, the amount of metal (e.g., by % weight, actual weight) can be at least 5 percent, e.g., at least 5, 10, 20, 30, 40, 50, 60, 70, 80, 90, 95, 99 percent, or more, e.g., w/w; less than 20 percent, e.g., less than 20, 15, 10, 5, 1, 0.5, 0.1 percent, or less.

In some embodiments, the implantable element (e.g., a device or material) is a ceramic. Exemplary ceramic materials include oxides, carbides, or nitrides of the transition elements, such as titanium oxides, hafnium oxides, iridium oxides, chromium oxides, aluminum oxides, and zirconium oxides. Silicon based materials, such as silica, may also be used. In ceramic-containing implantable elements, the amount of ceramic (e.g., by % weight, actual weight) can be at least 5 percent, e.g., at least 5, 10, 20, 30, 40, 50, 60, 70, 80, 90, 95, 99 percent, or more, e.g., w/w; less than 20 percent, e.g., less than 20, 15, 10, 5, 1, 0.5, 0.1 percent, or less.

In some embodiments, an implantable element comprises a polymer (e.g., hydrogel, plastic) component. Exemplary polymers include polyethylene, polypropylene, polystyrene, polyester (e.g., PLA, PLG, or PGA, polyhydroxyalkanoates (PHAs), or other biosorbable plastic), polycarbonate, polyvinyl chloride (PVC), polyethersulfone (PES), polyacrylate (e.g., acrylic or PMMA), hydrogel (e.g., acrylic polymer or blend of acrylic and silicone polymers), polysulfone, polyetheretherketone, thermoplastic elastomers (TPE or TPU), thermoset elastomer (e.g., silicone (e.g., silicone elastomer)), poly-p-xylylene (Parylene), fluoropolymers (e.g., PTFE), and polyacrylics such as poly(acrylic acid) and/or poly(acrylamide), or mixtures thereof. In polymer-containing implantable elements, the amount of polymer (e.g., by % weight, actual weight) can be at least 5 percent, e.g., at least 5, 10, 20, 30, 40, 50, 60, 70, 80, 90, 95, 99 percent or more, e.g., w/w; less than 20 percent, e.g., less than 20, 15, 10, 5, 1, 0.5, 0.1 percent, or less.

In some embodiments, the implantable element (e.g., a device or material) comprises a polymer that is (i) modified with a compound of Formula (I) and (ii) is covalently or non-covalently associated with a component of the implantable element (e.g., the surface of the implantable element). In some embodiments, the polymer is covalently associated with a component of the implantable element (e.g., on the inner surface or outer surface of an implantable element). In some embodiments, the polymer is non-covalently associated with a component of the implantable element (e.g., on the inner surface or outer surface of an implantable element). The polymer can be applied to an implantable element by a variety of techniques in the art including, but not limited to, spraying, wetting, immersing, dipping, such as dip coating (e.g., intraoperative dip coating), painting, or otherwise applying a hydrophobic polymer to a surface of the implantable element.

In an embodiment, the implantable element comprises a flexible polymer, e.g., alginate (e.g., any of the chemically modified alginates described herein), PLA, PLG, PEG, CMC, or mixtures thereof (referred to herein as a “polymer encapsulated implantable device”).

In some embodiments, the implantable element comprises a hydrogel-forming polymer. Hydrogel-forming polymers comprise a hydrophilic structure that renders them capable of holding large amounts of water in a three-dimensional network. Hydrogel-forming polymers may include polymers which form homopolymeric hydrogels, copolymeric hydrogels, or multipolymer interpenetrating polymeric hydrogels, and may be amorphous, semicrystalline, or crystalline in nature, e.g., as described in Ahmed (2015) J Adv Res 6:105-121. Exemplary hydrogel-forming polymers include proteins (e.g., collagen), gelatin, polysaccharides (e.g., starch, alginate, hyaluronate, agarose), and synthetic polymers. In some embodiments, the hydrogel-forming polymer is a polysaccharide (e.g., alginate).

In some embodiments, the implantable element comprises a polysaccharide. Exemplary polysaccharides include alginate, agar, agarose, carrageenan, hyaluronate, amylopectin, glycogen, gelatin, cellulose, amylose, chitin, chitosan, or a derivative or variant thereof, e.g., as described in Laurienzo (2010), Mar Drugs 9:2435-65. An implantable element may comprise a polysaccharide comprising heparin, chondoitin sulfate, dermatan, dextran, or carboxymethylcellulose. In some embodiments, a polysaccharide is a cross-linked polymer. In some embodiments, a polysaccharide is a cell-surface polysaccharide.

In some embodiments, the implantable element comprises an alginate. In some embodiments, the ratio of M:G in the alginate is about 1. In some embodiments, the ratio of M:G in the alginate is less than 1. In some embodiments, the ratio of M:G in the alginate is greater than 1. In some embodiments, the alginate is any of the modified alginates described herein.

In an embodiment, an implantable element comprises is formed, or could be formed, in situ on or surrounding cell, e.g., a plurality of cells, e.g., a cluster of cells, or on a microcarrier, e.g., a bead, or a matrix comprising cell or cells.

In an embodiment, an implantable element comprises is preformed prior to combination with the enclosed cell, e.g., a plurality of cells, e.g., a cluster of cells, or on a microcarrier, e.g., a bead, or a matrix comprising cell or cells. An implantable element can include a protein or polypeptide, e.g., an antibody, protein, enzyme, or growth factor. An implantable element can include an active or inactive fragment of a protein or polypeptide, such as a glucose oxidase (e.g., for glucose sensor), kinase, phosphatase, oxygenase, hydrogenase, or reductase.

Implantable elements included herein include implantable elements that are configured with a lumen, e.g., a lumen having one, two or more openings, e.g., tubular devices, e.g., a catheter. A typical stent is an example of a device configured with a lumen and having two openings. Other examples include shunts.

Implantable elements included herein include flexible implantable elements, e.g., that are configured to conform to the shape of the body.

Implantable elements included herein include components that stabilize the location of the implantable element, e.g., an adhesive, or fastener, e.g., a torque-based or friction-based fastener, e.g., a screw or a pin.

Implantable elements included herein may be configured to monitor a substance, e.g., an exogenous substance, e.g., a therapeutic agent or toxin, or an endogenous body product, e.g., a polypeptide e.g., insulin or glucose. In some embodiments, the implantable element is a diagnostic.

Implantable elements included herein may be configured to release a substance, e.g., an exogenous substance, e.g., a therapeutic agent described herein. In some embodiments, the therapeutic agent is a compound of Formula (I) or a pharmaceutically acceptable salt thereof. In some embodiments, the therapeutic agent is a biological material. In some embodiments, the therapeutic agent is a nucleic acid (e.g., an RNA or DNA), protein (e.g., a hormone, enzyme, antibody, antibody fragment, antigen, or epitope), small molecule, lipid, drug, vaccine, or any derivative thereof.

Implantable elements herein may be configured to change conformation in response to a signal or movement of the body, e.g., an artificial joint, e.g., a knee, hip, or other artificial joint.

Exemplary implantable elements include a stent, shunt, dressing, ocular device, port, sensor, orthopedic fixation device, implant (e.g., a dental implant, ocular implant, silicon implant, corneal implant, dermal implant, intragastric implant, facial implant, hip implant, bone implant, cochlear implant, penile implant, implants for control of incontinence), skin covering device, dialysis media, drug-delivery device, artificial or engineered organ (e.g., a spleen, kidney, liver, or heart), drainage device (e.g., a bladder drainage device), cell selection system, adhesive (e.g., a cement, clamp, clip), contraceptive device, intrauterine device, defibrillator, dosimeter, electrode, pump (e.g., infusion pump) filter, embolization device, fastener, fillers, fixative, graft, hearing aid, cardio or heart-related device (e.g., pacemaker, heart valve), battery or power source, hemostatic agent, incontinence device, intervertebral body fusion device, intraoral device, lens, mesh, needle, nervous system stimulator, patch, peritoneal access device, plate, plug, pressure monitoring device, ring, transponder, and valve. Also included are devices used in one or more of anesthesiology, cardiology, clinical chemistry, otolaryngology, dentistry, gastroenterology, urology, hematology, immunology, microbiology, neurology, obstetrics/gynecology, ophthalmology, orthopedic, pathology, physical medicine, radiology, general or plastic surgery, veterinary medicine, psychiatry, surgery, and/or clinical toxicology.

Implantable elements included herein include FDA class 1, 2, or 3 devices, e.g., devices that are unclassified or not classified, or classified as a humanitarian use device (HUD).

In some embodiments, an implantable element includes encapsulated or entrapped cells or tissues. The cells or tissue can be encapsulated or entrapped in a polymer. In some embodiments, an implantable element includes cells, e.g., cells disposed within a polymeric enclosing component (e.g., alginate).

In some embodiments, an implantable element targets or is designed for a certain system of the body, e.g. the nervous system (e.g., peripheral nervous system (PNS) or central nervous system (CNS)), vascular system, skeletal system, respiratory system, endocrine system, lymph system, reproductive system, or gastrointestinal tract. In some embodiments, an implantable element is targeted to the CNS. In some embodiments, an implantable element targets or is designed for a certain part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis.

Components or materials used in an implantable element (or the entire implantable element) can be optimized for one or more of biocompatibility (e.g., it minimizes immune rejection or fibrosis; heat-resistance; elasticity; tensile strength; chemical resistance (e.g., resistance to oils, greases, disinfectants, bleaches, processing aids, or other chemicals used in the production, use, cleaning, sterilizing and disinfecting of the device); electrical properties; surface and volume conductivity or resistivity, dielectric strength; comparative tracking index; mechanical properties; shelf life, long term durability sterilization capability (e.g., capable of withstanding sterilization processes, such as steam, dry heat, ethylene oxide (EtO), electron beam, and/or gamma radiation, e.g., while maintaining the properties for the intended use of the device), e.g., thermal resistance to autoclave/steam conditions, hydrolytic stability for steam sterilization, chemical resistance to EtO, resistance to high-energy radiation (e.g., electron beam, UV, and gamma); or crystal structure.

An implantable element can be assembled in vivo (e.g., injectable substance that forms a structured shape in vivo, e.g., at body temperature) or ex vivo.

An implantable element can have nanodimensions, e.g., can comprise a nanoparticle, e.g., nanoparticle made of a polymer described herein, e.g., PLA. Nanoparticles can be chemically modified nanoparticles, e.g., modified to prevent uptake by macrophages and Kupfer cells (e.g., a process called opsonization); or to alter the circulation half-life of the nanoparticle. Nanoparticles can include iron nanoparticle (injectable) (e.g., Advanced Magnetics iron nanoparticles). Exemplary nanoparticles are described in Veiseh et al (2010) Adv Drug Deliv Rev 62:284-304.

An implantable element can be configured for implantation in, administration to, or is administered to, implanted in or otherwise disposed into or onto any site of the body of a subject, including, but not limited to, the skin, a mucosal surface, a body cavity, intraperitoneal (IP) space, central nervous system (CNS) (e.g., brain or spinal cord), peripheral nervous system, an organ (e.g., heart, liver, kidney, bladder, pancreas, prostate, spleen, lung), lymphatic system, vasculature, oral cavity, nasal cavity, teeth, the gums, gastrointestinal tract, bone, hip, fat tissue (e.g., subcutaneous fat), muscle tissue, breast tissue, circulating blood, the eye, breast, vagina; uterus, a joint (e.g., in the knee, hip or spine): adjacent to a nerve, and a malignant or non-malignant tumor located on, in or near any of the foregoing.

In some embodiments, the implantable element is configured for implantation in, administration to, or is implanted or disposed into the IP space, e.g., within the peritoneal cavity, the omentumthe lesser sac. The lesser sac, also known as the omental bursa, refers to a cavity located in the abdomen formed by the omentum, and is in close proximity to, for example, the greater omentum, lesser omentum, stomach, small intestine, large intestine, liver, spleen, gastrosplenic ligament, adrenal glands, and pancreas. Typically, the lesser sac is connected to the greater sac via the omental foramen (i.e., the Foramen of Winslow). An implantable element may be implanted in or administered to the IP space, peritoneal cavity (e.g., the omentum, e.g., the lesser sac) or disposed on a surface within the peritoneal cavity (e.g., omentum, e.g., lesser sac) via injection or catheter. Additional considerations for implantation, administration or disposition of an implantable element into the omentum (e.g., the lesser sac) are provided in M. Pellicciaro et al. (2017) CellR4 5(3):e2410.

In some embodiments, the implantable element is configured for implantation in, administration to, or is implanted, administered or otherwise disposed into the CNS, e.g., the brain or spinal cord and their corresponding tissues and cavities, e.g., the dorsal body cavity, including the cranial cavity and the spinal canal. In some embodiments, the implantable element is configured for implantation in, administration to, or is implanted, administered to or otherwise disposed into an intracerebral space, e.g., the intraparenchymal space, the intraventricular space, or the subdural space. An implantable element may be implanted in the CNS or disposed on a surface within the CNS through a hole made in the skull and delivered via injection or catheter.

In some embodiments, the implantable element is configured for implantation in, administration to, or is implanted in, administered to or otherwise disposed into the eye, e.g., at one or more of the following: any surface or cavity within the eye, such as the retina, cornea, epithelium, aqueous humor, or vitreal space. An implantable element may be implanted in the eye or disposed on a surface within the eye through incision and/or injection.

An implantable element can comprise an electrochemical sensor, e.g., an electrochemical sensor including a working electrode and a reference electrode. For example, an electrochemical sensor includes a working electrode and a reference electrode that reacts with an analyte to generate a sensor measurement related to a concentration of the analyte in a fluid to which the eye-mountable device is exposed. The implantable element can comprise a window, e.g., of a transparent polymeric material having a concave surface and a convex surface a substrate, e.g., at least partially embedded in a transparent polymeric material. An implantable element can also comprise an electronics module including one or more of an antenna; and a controller electrically connected to the electrochemical sensor and the antenna, wherein the controller is configured to control the electrochemical sensor to obtain a sensor measurement related to a concentration of an analyte in a fluid to which the implantable element, e.g., an mountable implantable element is exposed and use the antenna to indicate the sensor measurement.

An implantable element may take any suitable shape, such as a sphere, spheroid, ellipsoid, disk, cylinder, torus, cube, stadiumoid, cone, pyramid, triangle, rectangle, square, or rod, or may comprise a curved or flat section. Any shaped, curved, or flat implantable element may be coated or chemically derivatized with an afibrotic compound (e.g., as shown in Table 2), a polymer modified with an afibrotic compound (e.g., as shown in Table 2), or a pharmaceutically acceptable salt thereof.

In some embodiments, an implantable element has a largest linear dimension (LLD), mean diameter or size that is 1 millimeter (mm) or smaller, or is within a range of 0.2 mm to 1 mm, e.g., any of 0.2, 0.3, 0.4, 0.5, 0.6, 0.7, 0.8, 0.9 or 1 mm. In some embodiments, an implantable element has an LLD, mean diameter or size that is greater than 0.5 mm, 1 mm, or 1.5 mm. In some embodiments, an implantable element described herein is in a size range of 1 mm to 8 mm, 1 mm to 6 mm, 1 mm to 5 mm, 1 mm to 4 mm, 1 mm to 3 mm, 1 mm to 2 mm, 1 mm to 1.5 mm, 1.5 mm to 8 mm, 1.5 mm to 6 mm, 1.5 mm to 5 mm, 1.5 mm to 4 mm, 1.5 mm 20 to 3 mm, 1.5 mm to 2 mm, 2 mm to 8 mm, 2 mm to 7 mm, 2 mm to 6 mm, 2 mm to 5 mm, 2 mm to 4 mm, 2 mm to 3 mm, 2.5 mm to 8 mm, 2.5 mm to 7 mm, 2.5 mm to 6 mm, 2.5 mm to 5 mm, 2.5 mm to 4 mm, 2.5 mm to 3 mm, 3 mm to 8 mm, 3 mm to 7 mm, 3 mm to 6 mm, 3 mm to 5 mm, 3 mm to 4 mm, 3.5 mm to 8 mm, 3.5 mm to 7 mm, 3.5 mm to 6 mm, 3.5 mm to 5 mm, 3.5 mm to 4 mm, 4 mm to 8 mm, 4 mm to 7 mm, 4 mm to 6 mm, 4 mm to 5 mm, 4.5 mm to 8 mm, 4.5 mm to 7 mm, 4.5 mm to 6 mm, 4.5 mm to 5 mm, 5 mm to 8 mm, 5 mm to 7 mm, 5 mm to 6 mm, 5.5 mm to 8 mm, 5.5 mm to 7 mm, 5.5 mm to 6 mm, 6 mm to 8 mm, 6 mm to 7 mm, 6.5 mm to 8 mm, 6.5 mm to 7 mm, 7 mm to 8 mm, or 7.5 mm to 8 mm. In some embodiments, the implantable element has an LLD, mean diameter or size of 0.5 mm to 1 mm or 1 mm to 4 mm. In some embodiments, the implantable element has an LLD, mean diameter or size 1 mm to 2 mm. In some embodiments, the implantable element has a spherical shape and a mean diameter within any of the foregoing numerical ranges.

In some embodiments, an implantable element comprises at least one pore or opening, e.g., to allow for the free flow of materials. In some embodiments, the mean pore size of an implantable element is between about 0.1 μm to about 10 μm. For example, the mean pore size may be between 0.1 μm to 10 μm, 0.1 μm to 5 μm, 0.1 μm to 2 μm, 0.15 μm to 10 μm, 0.15 μm to 5 μm, 0.15 μm to 2 μm, 0.2 μm to 10 μm, 0.2 μm to 5 μm, 0.25 μm to 10 μm, 0.25 μm to 5 μm, 0.5 μm to 10 μm, 0.75 μm to 10 μm, 1 μm to 10 μm, 1 μm to 5 μm, 1 μm to 2 μm, 2 μm to 10 μm, 2 μm to 5 μm, or 5 μm to 10 μm. In some embodiments, the mean pore size of an implantable element is between about 0.1 μm to 10 μm. In some embodiments, the mean pore size of an implantable element is between about 0.1 μm to 5 μm. In some embodiments, the mean pore size of an implantable element is between about 0.1 μm to 1 μm.

In some embodiments, an implantable element is capable of preventing materials over a certain size from passing through a pore or opening. In some embodiments, an implantable element is capable of preventing materials greater than 50 kD, 75 kD, 100 kD, 125 kD, 150 kD, 175 kD, 200 kD, 250 kD, 300 kD, 400 kD, 500 kD, 750 kD, 1,000 kD from passing through.

An implantable element (e.g., an implantable element described herein) may be provided as a preparation or composition for implantation or administration to a subject. In some embodiments, at least 20%, 30%, 35%, 40%, 45%, 50%, 55%, 60%, 65%, 70%, 75%, 80%, 85%, 90%, 95% or 100% of the implantable elements in a preparation or composition have a characteristic as described herein, e.g., mean pore size.

In some embodiments, an implantable element may be configured for or used for varying periods of time, ranging from a few minutes to several years. For example, an implantable element may be configured for or used from about 1 hour to about 10 years. In some embodiments, an implantable element is configured for, or is used for, longer than about any of the following time periods: 1 to 24 hours; 1 to 7 days; 1 to 4 weeks; 1 to 24 months; 2 to 10 years, or longer. An implantable element may be configured to function for the expected duration of implantation, e.g., configured to resist inactivation by PFO for all or part of the expected duration.

In some embodiments, the implantable element is easily retrievable from a subject, e.g., without causing injury to the subject or without causing significant disruption of the surrounding tissue. In an embodiment, the implantable element can be retrieved with minimal or no surgical separation of the implantable element from surrounding tissue, e.g., via minimally invasive surgical insection, extraction, or resection.

In some embodiments, the implantable element is not an implantable element disclosed in any of WO2012/112982, WO2012/167223, WO2014/153126, WO2016/187225, WO2016/019391, WO2017/075630, WO 2017/075631, WO 2018/067615, WO 2019/169333, or US 2016-0030359.

In some embodiments, an implantable element is associated with a polysaccharide polymer of Formula (I). In some embodiments, an implantable element comprises with a polysaccharide polymer of Formula (I) and a cell that is entirely or partially disposed within the implantable element.

In some embodiments, a surface of the implantable element comprising a cell (e.g., an engineered cell) is chemically modified with an afibrotic compound (e.g., as shown in Table 2). In some embodiments, a surface comprises an outer surface or an inner surface of the implantable element. In some embodiments, the surface (e.g., outer surface) of the implantable element comprising a cell (e.g., an engineered cell) is chemically modified with an afibrotic compound (e.g., as shown in Table 2).=. In some embodiments, the surface (e.g., outer surface) is covalently linked to an afibrotic compound (e.g., as shown in Table 2).

An implantable element may be coated an afibrotic compound (e.g., as shown in Table 2) or a pharmaceutically acceptable salt thereof, or a polymer comprising an afibrotic compound (e.g., as shown in Table 2) or a pharmaceutically acceptable salt thereof. In an embodiment, the afibrotic compound (e.g., as shown in Table 2) is disposed on a surface, e.g., an inner or outer surface, of the implantable element. In some embodiments, the afibrotic compound (e.g., as shown in Table 2) is disposed on a surface, e.g., an inner or outer surface, of an enclosing component associated with an implantable element. In an embodiment, the afibrotic compound (e.g., as shown in Table 2) is distributed evenly across a surface. In an embodiment, the afibrotic compound (e.g., as shown in Table 2) is distributed unevenly across a surface.

In some embodiments, an implantable element (e.g., or an enclosing component thereof) is coated (e.g., covered, partially or in full), with an afibrotic compound (e.g., as shown in Table 2) or a polymer modified with an afibrotic compound (e.g., as shown in Table 2) or a pharmaceutically acceptable salt thereof. In some embodiments, an implantable element (e.g., or an enclosing component thereof) is coated with a single layer of an afibrotic compound (e.g., as shown in Table 2). In some embodiments, an implantable element is coated with multiple layers of an afibrotic compound (e.g., as shown in Table 2), e.g., at least 2 layers, 3 layers, 4 layers, 5 layers, 10 layers, 20 layers, 50 layers or more.

In an embodiment, a first portion of the surface of the implantable element comprises a an afibrotic compound (e.g., as shown in Table 2) and a second portion of the implantable element lacks the compound, or has a substantially lower density of the compound.

In some embodiments, an implantable element is coated or chemically derivatized in a symmetrical manner with an afibrotic compound (e.g., as shown in Table 2), or a material comprising an afibrotic compound (e.g., as shown in Table 2), or a pharmaceutically acceptable salt thereof. In some embodiments, an implantable element is coated or chemically derivatized in an asymmetrical manner with an afibrotic compound (e.g., as shown in Table 2), or a polymer modified with an afibrotic compound (e.g., as shown in Table 2), or a pharmaceutically acceptable salt thereof. For example, an exemplary implantable element may be partially coated (e.g., at least about 5%, 10%, 15%, 20%, 25%, 30%, 35%, 40%, 45%, 50%, 55%, 60%, 65%, 70%, 75%, 80%, 85%, 90%, 95%, 99%, or 99.9% coated) with a compound of Formula (I) or a polymer modified with an afibrotic compound (e.g., as shown in Table 2) or a pharmaceutically acceptable salt thereof.

Exemplary implantable elements coated or chemically derivatized with an afibrotic compound (e.g., as shown in Table 2), or a polymer modified with an afibrotic compound (e.g., as shown in Table 2), or a pharmaceutically acceptable salt thereof may be prepared using any method known in the art, such as through self-assembly (e.g., via block copolymers, adsorption (e.g., competitive adsorption), phase separation, microfabrication, or masking).

In some embodiments, the implantable element comprises a surface exhibiting two or more distinct physicochemical properties (e.g., 3, 4, 5, 6, 7, 8, 9, 10, or more distinct physicochemical properties).

In some embodiments, the coating or chemical derivatization of the surface of an exemplary implantable element with an afibrotic compound (e.g., as shown in Table 2), a polymer modified with an afibrotic compound (e.g., as shown in Table 2), or a pharmaceutically acceptable salt thereof is described as the average number of attached compounds per given area, e.g., as a density. For example, the density of the coating or chemical derivatization of an exemplary implantable element may be 0.01, 0.1, 0.5, 1, 5, 10, 15, 20, 50, 75, 100, 200, 400, 500, 750, 1,000, 2,500, or 5,000 compounds per square μm or square mm, e.g., on the surface or interior of said implantable element.

An implantable element comprising an afibrotic compound (e.g., as shown in Table 2) or a pharmaceutically acceptable salt thereof may have a reduced immune response (e.g., a marker of an immune response) compared to an otherwise identical implantable element that does not comprise an afibrotic compound (e.g., as shown in Table 2) or a pharmaceutically acceptable salt thereof. A marker of immune response is one or more of: PFO, cathepsin level or the level of a marker of immune response, e.g., TNF-α, IL-13, IL-6, G-CSF, GM-CSF, IL-4, CCL2, or CCL4, as measured, e.g., by ELISA. In some embodiments, the immune response to an implantable element comprising an afibrotic compound (e.g., as shown in Table 2) or a pharmaceutically acceptable salt thereof is reduced by at least about 1 percent and up to about 100 percent, e.g., about 5, 10, 15, 20, 25, 30, 35, 40, 45, 50, 55, 60, 65, 70, 75, 80, 85, 90, 95, or 99 percent. In some embodiments, the reduced immune response (e.g., a marker of an immune response) is measured after about any of 30 minutes, 1 hour, 6 hours, 12 hours, about any of 1 day, 2 days, 3 days or 4 days, about 1 week or 2 weeks, about any of 1 month, 2 months, 3 months, 6 months or longer. In some embodiments, an implantable element comprising an afibrotic compound (e.g., as shown in Table 2) is coated by an afibrotic compound (e.g., as shown in Table 2) or encapsulated in a layer (e.g., a polymeric layer) comprising an afibrotic compound (e.g., as shown in Table 2).

An implantable element may have a smooth surface, or may comprise a protuberance, depression, well, slit, or hole, or any combination thereof. Said protuberance, depression, well, slit or hole may be any size, e.g., from 10 μm to about 1 nm, about 5 μm to about 1 nm, about 2.5 μm to about 1 nm, 1 μm to about 1 nm, 500 nm to about 1 nm, or about 100 nm to about 1 nm. The smooth surface or protuberance, depression, well, slit, or hole, or any combination thereof, may be coated or chemically derivatized with an afibrotic compound (e.g., as shown in Table 2), polymer modified with an afibrotic compound (e.g., as shown in Table 2), or a pharmaceutically acceptable salt thereof.

In an embodiment, an implantable element comprises any of the polymers described herein, modified with an afibrotic compound (e.g., as shown in Table 2) or a pharmaceutically acceptable salt thereof.

In some embodiments, the implantable element comprises between 5 to 50% of an afibrotic compound (e.g., as shown in Table 2), e.g., as measured using a quantative amine assay. In some embodiments, the implantable element comprises between 10 to 50% of an afibrotic compound (e.g., as shown in Table 2), e.g., 15 to 45% of an afibrotic compound (e.g., as shown in Table 2), 15 to 40% of an afibrotic compound (e.g., as shown in Table 2), 15 to 35% of an afibrotic compound (e.g., as shown in Table 2), 15 to 30% of an afibrotic compound (e.g., as shown in Table 2), 20 to 45% of an afibrotic compound (e.g., as shown in Table 2), 20 to 40% of an afibrotic compound (e.g., as shown in Table 2), 20 to 35% of an afibrotic compound (e.g., as shown in Table 2), or 20 to 30% of an afibrotic compound (e.g., as shown in Table 2), as measured using a quantative amine assay.

In some embodiments, an implantable element comprises an alginate (e.g., any of the alginates described herein) modified with an afibrotic compound (e.g., as shown in Table 2) In some embodiments, an implantable element comprises an alginate modified with a compound shown in Table 2. In some embodiments, an implantable element comprises an alginate modified with Compound 200. In some embodiments, an implantable element comprises an alginate modified with Compound 218. In some embodiments, an implantable element comprises an alginate modified with Compound 219. In some embodiments, an implantable element comprises an alginate modified with Compound 224. In some embodiments, an implantable element comprises an alginate modified with Compound 222.

### Cells and Therapeutic Agents

The implantable elements of the present disclosure may comprise a wide variety of different cell types (e.g., human cells), including but not limited to: adipose cells, epidermal cells, epithelial cells, endothelial cells, fibroblast cells, islet cells, mesenchymal stem cells, pericytes, subtypes of any of the foregoing, cells derived from any of the foregoing, cells derived from induced pluripotent stem cells and mixtures of one or more of any of the foregoing. Exemplary cell types include the cell types recited in WO 2017/075631 and WO 2019/195055. In an embodiment, the implantable elements described herein comprise a plurality of cells. In an embodiment, the plurality of cells is in the form of a cell suspension prior to being encapsulated within an implantable element described herein. The cells in the suspension may take the form of single cells (e.g., from a monolayer cell culture), or provided in another form, e.g., disposed on a microcarrier (e.g., a bead or matrix) or as a three-dimensional aggregate of cells (e.g., a cell cluster or spheroid). The cell suspension can comprise multiple cell clusters (e.g., as spheroids) or microcarriers. In some embodiments, the device does not comprise any islet cells and does not comprise any cells that are capable of producing insulin in a glucose-responsive manner.

The present disclosure features a cell that produces or is capable of producing a therapeutic agent for the prevention or treatment of a disease, disorder, or condition described herein. In an embodiment, the cell is an engineered cell. In an embodiment, the cell is engineered to sense a stimulus, e.g., a chemical signal, and express the therapeutic agent in response to the stimulus. The therapeutic agent may be any biological substance, such as a nucleic acid (e.g., a nucleotide, DNA, or RNA), a polypeptide, a lipid, a sugar (e.g., a monosaccharide, disaccharide, oligosaccharide, or polysaccharide), or a small molecule, each of which are further elaborated below. Exemplary therapeutic agents include the agents listed in WO 2017/075631 and WO 2019/195055.

In some embodiments, the cells (e.g., engineered cells) produce a nucleic acid. A nucleic acid produced by a cell described herein may vary in size and contain one or more nucleosides or nucleotides, e.g., greater than 2, 3, 4, 5, 10, 25, 50, or more nucleosides or nucleotides. In some embodiments, the nucleic acid is a short fragment of RNA or DNA, e.g., and may be used as a reporter or for diagnostic purposes. Exemplary nucleic acids include a single nucleoside or nucleotide (e.g., adenosine, thymidine, cytidine, guanosine, uridine monophosphate, inosine monophosphate), RNA (e.g., mRNA, siRNA, miRNA, RNAi), and DNA (e.g., a vector, chromosomal DNA). In some embodiments, the nucleic acid has an average molecular weight (in kD) of about 0.25, 0.5, 1, 1.5, 2, 2.5, 5, 10, 25, 50, 100, 150, 200 or more.

In some embodiments, the therapeutic agent is a peptide or polypeptide (e.g., a protein), such as a hormone, enzyme, cytokine (e.g., a pro-inflammatory cytokine or an anti-inflammatory cytokine), growth factor, clotting factor, or lipoprotein. A peptide or polypeptide (e.g., a protein, e.g., a hormone, growth factor, clotting factor or coagulation factor, antibody molecule, enzyme, cytokine, cytokine receptor, or a chimeric protein including cytokines or a cytokine receptor) produced by a cell in an implantable element can have a naturally occurring amino acid sequence, or may contain a variant of the naturally occurring sequence. The variant can be a naturally occurring or non-naturally occurring amino acid substitution, mutation, deletion or addition relative to the reference naturally occurring sequence. The naturally occurring amino acid sequence may be a polymorphic variant. The naturally occurring amino acid sequence can be a human or a non-human amino acid sequence. In some embodiments, the naturally occurring amino acid sequence or naturally occurring variant thereof is a human sequence. In addition, a peptide or polypeptide (e.g., a protein) for use with the present invention may be modified in some way, e.g., via chemical or enzymatic modification (e.g., glycosylation, phosphorylation). In some embodiments, the peptide has about 2, 3, 4, 5, 6, 7, 8, 9, 10, 12, 14, 16, 18, 20, 25, 30, 35, 40, 45, or 50 amino acids. In some embodiments, the protein has an average molecular weight (in kD) of 5, 10, 25, 50, 100, 150, 200, 250, 500 or more.

In some embodiments, the protein is a hormone. Exemplary hormones include anti-diuretic hormone (ADH), oxytocin, growth hormone (GH), prolactin, growth hormone-releasing hormone (GHRH), thyroid stimulating hormone (TSH), thyrotropin-release hormone (TRH), adrenocorticotropic hormone (ACTH), follicle-stimulating hormone (FSH), luteinizing hormone (LH), luteinizing hormone-releasing hormone (LHRH), thyroxine, calcitonin, parathyroid hormone, aldosterone, cortisol, epinephrine, glucagon, insulin, estrogen, progesterone, and testosterone. In some embodiments, the protein is insulin (e.g., insulin A-chain, insulin B-chain, or proinsulin). In some embodiments, the protein is a growth hormone, such as human growth hormone (hGH), recombinant human growth hormone (rhGH), bovine growth hormone, methione-human growth hormone, des-phenylalanine human growth hormone, and porcine growth hormone. In some embodiments, the protein is not insulin (e.g., insulin A-chain, insulin B-chain, or proinsulin).

In some embodiments, the protein is a growth factor, e.g., vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

In some embodiments, the protein is a clotting factor or a coagulation factor, e.g., a blood clotting factor or a blood coagulation factor. In some embodiments, the protein is a protein involved in coagulation, i.e., the process by which blood is converted from a liquid to solid or gel. Exemplary clotting factors and coagulation factors include Factor I (e.g., fibrinogen), Factor II (e.g., prothrombin), Factor III (e.g., tissue factor), Factor V (e.g., proaccelerin, labile factor), Factor VI, Factor VII (e.g., stable factor, proconvertin), Factor VIII (e.g., antihemophilic factor A), Factor VIIIC, Factor IX (e.g., antihemophilic factor B), Factor X (e.g., Stuart-Prower factor), Factor XI (e.g., plasma thromboplastin antecedent), Factor XII (e.g., Hagerman factor), Factor XIII (e.g., fibrin-stabilizing factor), von Willebrand factor, prekallikrein, heparin cofactor II, high molecular weight kininogen (e.g., Fitzgerald factor), antithrombin III, and fibronectin. In some embodiments, the protein is an anti-clotting factor, such as Protein C.

In some embodiments, the protein is an antibody molecule. As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full-length antibody which has an immunoglobulin Fc region). In an embodiment, an antibody molecule comprises a full-length antibody, or a full-length immunoglobulin chain. In an embodiment, an antibody molecule comprises an antigen binding or functional fragment of a full-length antibody, or a full-length immunoglobulin chain. In an embodiment, an antibody molecule is a monospecific antibody molecule and binds a single epitope, e.g., a monospecific antibody molecule having a plurality of immunoglobulin variable domain sequences, each of which binds the same epitope. In an embodiment, an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

Various types of antibody molecules may be produced by a cell in an implantable element described herein, including whole immunoglobulins of any class, fragments thereof, and synthetic proteins containing at least the antigen binding variable domain of an antibody. The antibody molecule can be an antibody, e.g., an IgG antibody, such as IgG₁, IgG₂, IgG₃, or IgG₄. An antibody molecule can be in the form of an antigen binding fragment including a Fab fragment, F(ab′)2 fragment, a single chain variable region, and the like. Antibodies can be polyclonal or monoclonal (mAb). Monoclonal antibodies may include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity. In some embodiments, the antibody molecule is a single-domain antibody (e.g., a nanobody). The described antibodies can also be modified by recombinant means, for example by deletions, additions or substitutions of amino acids, to increase efficacy of the antibody in mediating the desired function. Exemplary antibodies include anti-beta-galactosidase, anti-collagen, anti-CD14, anti-CD20, anti-CD40, anti-HER2, anti-IL-1, anti-IL-4, anti-IL6, anti-IL-13, anti-IL17, anti-IL18, anti-IL-23, anti-IL-28, anti-IL-29, anti-IL-33, anti-EGFR, anti-VEGF, anti-CDF, anti-flagellin, anti-IFN-α, anti-IFN-β, anti-IFN-γ, anti-mannose receptor, anti-VEGF, anti-TLR1, anti-TLR2, anti-TLR3, anti-TLR4, anti-TLR5, anti-TLR6, anti-TLR9, anti-PDF, anti-PD1, anti-PDL-1, or anti-nerve growth factor antibody. In some embodiments, the antibody is an anti-nerve growth factor antibody (e.g., fulranumab, fasinumab, tanezumab).

In some embodiments, the protein is a cytokine or a cytokine receptor, or a chimeric protein including cytokines or their receptors, including, for example tumor necrosis factor alpha and beta, their receptors and their derivatives, renin; lipoproteins; colchicine; corticotrophin; vasopressin; somatostatin; lypressin; pancreozymin; leuprolide; alpha-1-antitrypsin; atrial natriuretic factor; lung surfactant; a plasminogen activator other than a tissue-type plasminogen activator (t-PA), for example a urokinase; bombesin; thrombin; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; a microbial protein, such as beta-lactamase; DNase; inhibin; activin; receptors for hormones or growth factors; integrin; protein A or D; rheumatoid factors; platelet-derived growth factor (PDGF); epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; an interferon such as interferon-alpha (e.g., interferon.alpha.2A), -beta, -gamma, -lambda and consensus interferon; colony stimulating factors (CSFs), e.g., M-CSF, GM-CSF, and G-CSF; interleukins (TLs), e.g., IL-1 to IL-10; superoxide dismutase; T-cell receptors; surface membrane proteins; decay accelerating factor; transport proteins; homing receptors; addressins; fertility inhibitors such as the prostaglandins; fertility promoters; regulatory proteins; antibodies (including fragments thereof) and chimeric proteins, such as immunoadhesins; precursors, derivatives, prodrugs and analogues of these compounds, and pharmaceutically acceptable salts of these compounds, or their precursors, derivatives, prodrugs and analogues. Suitable proteins or peptides may be native or recombinant and include, e.g., fusion proteins.

Examples of a polypeptide (e.g., a protein) produced by a cell in an implantable element described herein also include CCL1, CCL2 (MCP-1), CCL3 (MIP-1α), CCL4 (MIP-1β), CCL5 (RANTES), CCL6, CCL7, CCL8, CCL9 (CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1 (KC), CXCL2 (SDF1a), CXCL3, CXCL4, CXCL5, CXCL6, CXCL7, CXCL8 (IL8), CXCL9, CXCL10, CXCL11, CXCL12, CXCL13, CXCL14, CXCL15, CXCL16, CXCL17, CX3CL1, XCL1, XCL2, TNFA, TNFB (LTA), TNFC (LTB), TNFSF4, TNFSF5 (CD40LG), TNFSF6, TNFSF7, TNFSF8, TNFSF9, TNFSF10, TNFSF11, TNFSF13B, EDA, IL2, IL15, IL4, IL13, IL7, IL9, IL21, IL3, ILS, IL6, IL11, IL27, IL30, IL31, OSM, LIF, CNTF, CTF1, IL12a, IL12b, IL23, IL27, IL35, IL14, IL16, IL32, IL34, IL10, IL22, IL19, IL20, IL24, IL26, IL29, IFNL1, IFNL2, IFNL3, IL28, IFNA1, IFNA2, IFNA4, IFNA5, IFNA6, IFNA7, IFNA8, IFNA10, IFNA13, IFNA14, IFNA16, IFNA17, IFNA21, IFNB1, IFNK, IFNW1, IFNG, ILlA (IL1F1), IL1B (IL1F2), IL1Ra (IL1F3), IL1F5 (IL36RN), IL1F6 (IL36A), IL1F7 (IL37), IL1F8 (IL36B), IL1F9 (IL36G), IL1F10 (IL38), IL33 (IL1F11), IL18 (IL1G), IL17, KITLG, IL25 (IL17E), CSF1 (M-CSF), CSF2 (GM-CSF), CSF3 (G-CSF), SPP1, TGFB1, TGFB2, TGFB3, CCL3L1, CCL3L2, CCL3L3, CCL4L1, CCL4L2, IL17B, IL17C, IL17D, IL17F, AIMP1 (SCYE1), MIF, Areg, BC096441, Bmp1, Bmp10, Bmp15, Bmp2, Bmp3, Bmp4, Bmp5, Bmp6, Bmp7, Bmp8a, Bmp8b, C1qtnf4, Ccl21a, Ccl27a, Cd70, Cer1, Cklf, Clcf1, Cmtm2a, Cmtm2b, Cmtm3, Cmtm4, Cmtm5, Cmtm6, Cmtm7, Cmtm8, Crlfl, Ctf2, Ebi3, Edn1, Fam3b, Fasl, Fgf2, Flt31, Gdf10, Gdf11, Gdf15, Gdf2, Gdf3, Gdf5, Gdf6, Gdf7, Gdf9, Gm12597, Gm13271, Gm13275, Gm13276, Gm13280, Gm13283, Gm2564, Gpi1, Grem1, Grem2, Grn, Hmgb1, Ifnal1, Ifna12, Ifna9, Ifnab, Ifne, Il17a, Il23a, 1125, 1131, Iltifb, Inhba, Lefty1, Lefty2, Mstn, Nampt, Ndp, Nodal, Pf4, Pglyrp1, Prl7d1, Scg2, Scgb3a1, Slurp1, Spp1, Thpo, Tnfsf10, Tnfsf11, Tnfsf12, Tnfsf13, Tnfsf13b, Tnfsf14, Tnfsf15, Tnfsf18, Tnfsf4, Tnfsf8, Tnfsf9, Tslp, Vegfa, Wnt1, Wnt2, Wnt5a, Wnt7a, Xcl1, epinephrine, melatonin, triiodothyronine, a prostaglandin, a leukotriene, prostacyclin, thromboxane, islet amyloid polypeptide, mullerian inhibiting factor or hormone, adiponectin, corticotropin, angiotensin, vasopressin, arginine vasopressin, atriopeptin, brain natriuretic peptide, calcitonin, cholecystokinin, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, hepcidin, human chorionic gonadotropin, human placental lactogen, inhibin, somatomedin, leptin, lipotropin, melanocyte stimulating hormone, motilin, orexin, oxytocin, pancreatic polypeptide, pituitary adenylate cyclase-activating peptide, relaxin, renin, secretin, somatostatin, thrombopoietin, thyrotropin, thyrotropin-releasing hormone, vasoactive intestinal peptide, androgen, alpha-glucosidase (also known as acid maltase), glycogen phosphorylase, glycogen debrancher enzyme, phosphofructokinase, phosphoglycerate kinase, phosphoglycerate mutase, lactate dehydrogenase, carnitine palymityl transferase, carnitine, and myoadenylate deaminase.

In some embodiments, the protein is a replacement therapy or a replacement protein. In some embodiments, the replacement therapy or replacement protein is a clotting factor or a coagulation factor, e.g., Factor VIII (e.g., comprises a naturally occurring human Factor VIII amino acid sequence or a variant thereof) or Factor IX (e.g., comprises a naturally occurring human Factor IX amino acid sequence or a variant thereof).

In some embodiments, the cell is engineered to express a Factor VIII, e.g., a recombinant Factor VIII. In some embodiments, the cell is derived from human tissue and is engineered to express a Factor VIII, e.g., a recombinant Factor VIII. In some embodiments, the recombinant Factor VIII is a B-domain-deleted recombinant Factor VIII (FVIII-BDD).

In some embodiments, the cell is derived from human tissue and is engineered to express a Factor IX, e.g., a recombinant Factor IX. In some embodiments, the cell is engineered to express a Factor IX, e.g., a wild-type human Factor IX (FIX), or a polymorphic variant thereof. In some embodiments, the cell is engineered to express a gain-in-function (GIF) variant of a wild-type FIX protein (FIX-GIF), wherein the GIF variant has higher specific activity than the corresponding wild-type FIX.

In some embodiments, the replacement therapy or replacement protein is an enzyme, e.g., alpha-galactosidase, alpha-L-iduronidase (IDUA), or N-sulfoglucosamine sulfohydrolase (SGSH). In some embodiments, the replacement therapy or replacement protein is an enzyme, e.g., an alpha-galactosidase A (e.g., comprises a naturally-occurring human alpha-galactosidase A amino acid sequence or a variant thereof). In some embodiments, the replacement therapy or replacement protein is a cytokine or an antibody.

In some embodiments, the therapeutic agent is a sugar, e.g., monosaccharide, disaccharide, oligosaccharide, or polysaccharide. In some embodiments, a sugar comprises a triose, tetrose, pentose, hexose, or heptose moiety. In some embodiments, the sugar comprises a a linear monosaccharide or a cyclized monosaccharide. In some embodiments, the sugar comprises a glucose, galactose, fructose, rhamnose, mannose, arabinose, glucosamine, galactosamine, sialic acid, mannosamine, glucuronic acid, galactosuronic acid, mannuronic acid, or guluronic acid moiety. In some embodiments, the sugar is attached to a protein (e.g., an N-linked glycan or an O-linked glycan). Exemplary sugars include glucose, galactose, fructose, mannose, rhamnose, sucrose, ribose, xylose, sialic acid, maltose, amylose, inulin, a fructooligosaccharide, galactooligosaccharide, a mannan, a lectin, a pectin, a starch, cellulose, heparin, hyaluronic acid, chitin, amylopectin, or glycogen. In some embodiments, the therapeutic agent is a sugar alcohol.

In some embodiments, the therapeutic agent is a lipid. A lipid may be hydrophobic or amphiphilic, and may form a tertiary structure such as a liposome, vesicle, or membrane or insert into a liposome, vesicle, or membrane. A lipid may comprise a fatty acid, glycerolipid, glycerophospholipid, sterol lipid, prenol lipid, sphingolipid, saccharolipid, polyketide, or sphingolipid. Examples of lipids produced by a cell described herein include anandamide, docosahexaenoic acid, aprostaglandin, a leukotriene, a thromboxane, an eicosanoid, a triglyceride, a cannabinoid, phosphatidylcholine, phosphatidylethanolamine, a phosphatidylinositol, a phosohatidic acid, a ceramide, a sphingomyelin, a cerebroside, a ganglioside, estrogen, androsterone, testosterone, cholesterol, a carotenoid, a quinone, a hydroquinone, or a ubiquinone.

In some embodiments, the therapeutic agent is a small molecule. A small molecule may include a natural product produced by a cell. In some embodiments, the small molecule has poor availability or does not comply with the Lipinski rule of five (a set of guidelines used to estimate whether a small molecule will likely be an orally active drug in a human; see, e.g., Lipinski, C. A. et al (2001) Adv Drug Deliv 46:2-36). Exemplary small molecule natural products include an anti-bacterial drug (e.g., carumonam, daptomycin, fidaxomicin, fosfomycin, ispamicin, micronomicin sulfate, miocamycin, mupiocin, netilmicin sulfate, teicoplanin, thienamycin, rifamycin, erythromycin, vancomycin), an anti-parasitic drug (e.g., artemisinin, ivermectin), an anticancer drug (e.g., doxorubicin, aclarubicin, aminolaevulinic acid, arglabin, omacetaxine mepesuccinate, paclitaxel, pentostatin, peplomycin, romidepsin, trabectdin, actinomycin D, bleomycin, chromomycin A, daunorubicin, leucovorin, neocarzinostatin, streptozocin, trabectedin, vinblastine, vincristine), anti-diabetic drug (e.g., voglibose), a central nervous system drug (e.g., L-dopa, galantamine, zicontide), a statin (e.g., mevastatin), an anti-fungal drug (e.g., fumagillin, cyclosporin), 1-deoxynojirimycin, and theophylline, sterols (cholesterol, estrogen, testosterone). Additional small molecule natural products are described in Newman, D. J. and Cragg, M. (2016) J Nat Prod 79:629-661 and Butler, M. S. et al (2014) Nat Prod Rep 31:1612-1661.

In some embodiments, the cell is engineered to synthesize a non-protein or non-peptide small molecule. For example, in an embodiment a cell can produce a statin (e.g., taurostatin, pravastatin, fluvastatin, or atorvastatin).

In some embodiments, the therapeutic agent is an antigen (e.g., a viral antigen, a bacterial antigen, a fungal antigen, a plant antigen, an environmental antigen, or a tumor antigen). An antigen is recognized by those skilled in the art as being immunostimulatory, i.e., capable of stimulating an immune response or providing effective immunity to the organism or molecule from which it derives. An antigen may be a nucleic acid, peptide, protein, sugar, lipid, or a combination thereof.

The cells, e.g., engineered cells, e.g., engineered cells described herein, may produce a single therapeutic agent or a plurality of therapeutic agents. In some embodiments, the cells produce a single therapeutic agent. In some embodiments, a cluster of cells comprises cells that produce a single therapeutic agent. In some embodiments, at least about 1 percent, or about 5, 10, 20, 25, 30, 40, 50, 60, 70, 80, 90, 95, or 99 percent of the cells in a cluster produce a single therapeutic agent (e.g., a therapeutic agent described herein). In some embodiments, the cells produce a plurality of therapeutic agents, e.g., at least 2, 3, 4, 5, 6, 7, 8, 9, or 10 therapeutic agents. In some embodiments, a cluster of cells comprises cells that produce a plurality of therapeutic agents. In some embodiments, at least about 1 percent, or about 5, 10, 20, 25, 30, 40, 50, 60, 70, 80, 90, 95, or 99 percent of the cells in a cluster produce a plurality of therapeutic agents (e.g., a therapeutic agent described herein).

The therapeutic agents may be related or may form a complex. In some embodiments, the therapeutic agent secreted or released from a cell in an active form. In some embodiments, the therapeutic agent is secreted or released from a cell in an inactive form, e.g., as a prodrug. In the latter instance, the therapeutic agent may be activated by a downstream agent, such as an enzyme. In some embodiments, the therapeutic agent is not secreted or released from a cell, but is maintained intracellularly. For example, the therapeutic agent may be an enzyme involved in detoxification or metabolism of an unwanted substance, and the detoxification or metabolism of the unwanted substance occurs intracellularly.

### Methods of Treatment

Described herein are methods for preventing or treating a disease, disorder, or condition in a subject through administration or implantation of an implantable element comprising a compound of Formula (I) or a pharmaceutically acceptable salt thereof. In some embodiments, the methods described herein directly or indirectly reduce or alleviate at least one symptom of a disease, disorder, or condition. In some embodiments, the methods described herein prevent or slow the onset of a disease, disorder, or condition. In some embodiments, the subject is a human.

In some embodiments, the disease, disorder, or condition affects a system of the body, e.g. the nervous system (e.g., peripheral or central nervous system), vascular system, skeletal system, respiratory system, endocrine system, lymph system, reproductive system, or gastrointestinal tract. In some embodiments, the disease, disorder, or condition affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis.

In some embodiments, the disease, disorder or condition is a neurodegenerative disease, diabetes (Type 1 or Type 2), a heart disease, an autoimmune disease, a cancer, a liver disease, a lysosomal storage disease, a blood clotting disorder or a coagulation disorder, an orthopedic condition, an amino acid metabolism disorder.

In some embodiments, the disease, disorder or condition is a neurodegenerative disease. Exemplary neurodegenerative diseases include Alzheimer's disease, Huntington's disease, Parkinson's disease (PD) amyotrophic lateral sclerosis (ALS), multiple sclerosis (MS) and cerebral palsy (CP), dentatorubro-pallidoluysian atrophy (DRPLA), neuronal intranuclear hyaline inclusion disease (NIHID), dementia with Lewy bodies, Down's syndrome, Hallervorden-Spatz disease, prion diseases, argyrophilic grain dementia, cortocobasal degeneration, dementia pugilistica, diffuse neurofibrillary tangles, Gerstmann-Straussler-Scheinker disease, Jakob-Creutzfeldt disease, Niemann-Pick disease type 3, progressive supranuclear palsy, subacute sclerosing panencephalitis, spinocerebellar ataxias, Pick's disease, and dentatorubral-pallidoluysian atrophy.

In some embodiments, the disease, disorder, or condition is an autoimmune disease, e.g., scleroderma, multiple sclerosis, lupus, or allergies.

In some embodiments, the disease is a liver disease, e.g., hepatitis B, hepatitis C, cirrhosis, NASH.

In some embodiments, the disease, disorder, or condition is cancer. Exemplary cancers include leukemia, lymphoma, melanoma, lung cancer, brain cancer (e.g., glioblastoma), sarcoma, pancreatic cancer, renal cancer, liver cancer, testicular cancer, prostate cancer, or uterine cancer.

In some embodiments, the disease, disorder, or condition is an orthopedic condition. Exemplary orthopedic conditions include osteoporosis, osteonecrosis, Paget's disease, or a fracture.

In some embodiments, the disease, disorder or condition is a lysosomal storage disease. Exemplary lysosomal storage diseases include Gaucher disease (e.g., Type I, Type II, Type III), Tay-Sachs disease, Fabry disease, Farber disease, Hurler syndrome (also known as mucopolysaccharidosis type I (MPS I)), Hunter syndrome, lysosomal acid lipase deficiency, Niemann-Pick disease, Salla disease, Sanfilippo syndrome (also known as mucopolysaccharidosis type IIIA (MPS3A)), multiple sulfatase deficiency, Maroteaux-Lamy syndrome, metachromatic leukodystrophy, Krabbe disease, Scheie syndrome, Hurler-Scheie syndrome, Sly syndrome, hyaluronidase deficiency, Pompe disease, Danon disease, gangliosidosis, or Morquio syndrome.

In some embodiments, the disease, disorder, or condition is a blood clotting disorder or a coagulation disorder. Exemplary blood clotting disorders or coagulation disorders include hemophilia (e.g., hemophilia A or hemophilia B), Von Willebrand diaease, thrombocytopenia, uremia, Bernard-Soulier syndrome, Factor XII deficiency, vitamin K deficiency, or congenital afibrinogenimia.

In some embodiments, the disease, disorder, or condition is an amino acid metabolism disorder, e.g., phenylketonuria, tyrosinemia (e.g., Type 1 or Type 2), alkaptonuria, homocystinuria, hyperhomocysteinemia, maple syrup urine disease.

In some embodiments, the disease, disorder, or condition is a fatty acid metabolism disorder, e.g., hyperlipidemia, hypercholesterolemia, galactosemia.

In some embodiments, the disease, disorder, or condition is a purine or pyrimidine metabolism disorder, e.g., Lesch-Nyhan syndrome.

In some embodiments, the disease, disorder, or condition is not Type I diabetes and/or is not Type II diabetes.

The present invention further comprises methods for identifying a subject having or suspected of having a disease, disorder, or condition described herein, and upon such identification, administering to the subject implantable element comprising a cell, e.g., optionally encapsulated by an enclosing component, and optionally modified with a compound of Formula (I) as described herein, or a composition thereof. In an embodiment, the subject is a human.

### ENUMERATED EMBODIMENTS

1. A polysaccharide polymer comprising a saccharide monomer, wherein the saccharide monomer comprises a hydroxyl-modifying agent covalently bound to a hydroxyl moiety.
2. The polysaccharide polymer of embodiment 1, wherein the hydroxyl-modifying agent comprises an alkyl, alkenyl, alkynyl, heteroalkyl, alkoxy, amine, amide, haloalkyl, haloalkoxy, ester, ether, carbamate, aryl, heteroaryl, cycloalkyl, or heterocyclyl moiety.
3. The polysaccharide polymer of any one of embodiments 1-2, wherein the polysaccharide polymer is selected from hyaluronate, alginate, cellulose, chitosan, chitin, amylose, dextran, starch, glycogen, chondroitin, and pectin.
4. The polysaccharide polymer of any one of embodiments 1-3, wherein the saccharide monomer is selected from glucose, galactose, mannose, allose, altrose, talose, idose, gulose, fructose, ribose, arabinose, lyxose, xylose, rhamnose, glucuronic acid, galacturonic acid, mannuronic acid, and guluronic acid.
5. The polysaccharide polymer of any one of embodiments 1-4, wherein the saccharide monomer is mannuronic acid or guluronic acid.
6. The polysaccharide polymer of any one of embodiments 1-5, wherein the saccharide monomer has a structure of Formula (I):

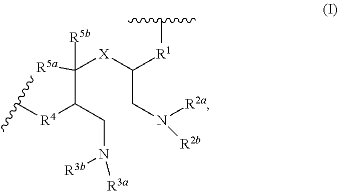

or a pharmaceutically acceptable salt thereof, wherein:
X is O, NR⁶, or S;
each of R¹ and R⁴ is independently absent, alkylene, alkenylene, alkynylene, heteroalkylene, haloalkylene, —N(R^{7a})—, —O—, —C(O)—, —C(O)O—, —C(O)N(R^{C})—, or —N(R^{c})C(O)—, wherein alkylene, alkenylene, alkynylene, heteroalkylene, and haloalkylene is optionally substituted by one or more R⁸;
R^{2a}, R^{2b}, R^{3a}, and R^{3b} are each independently hydrogen, alkyl, heteroalkyl, haloalkyl, C(O)R^{B}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted by one or more R⁹, wherein at least one of R^{2a} and R^{2b} and at least one of R^{3a} and R^{3b} is not hydrogen;
each of R^{5a} and R^{5b} is independently hydrogen, alkyl, alkenyl, alkynyl, heteroalkyl, haloalkyl, N(R^{7a})(R^{7b}), OR^{A}, C(O)R^{B}, C(O)OR^{A}, C(O)N(R^{C})(R^{D}), N(R^{C})C(O)R^{B}, halogen, cyano, azido, aryl, heteroaryl, cycloalkyl, heterocyclyl, an afibrotic compound, or a peptide, wherein alkyl, alkenyl, alkynyl, heteroalkyl, haloalkyl, aryl, heteroaryl, cycloalkyl, and heterocyclyl is optionally substituted by one or more R⁸;
R⁶ is hydrogen, alkyl, heteroalkyl, or haloalkyl, wherein alkyl, heteroalkyl, and haloalkyl is optionally substituted by one or more R⁹;
R^{7a} and R^{7b} are each independently hydrogen, alkyl, cycloalkyl, or heterocyclyl, wherein alkyl, cycloalkyl, or heterocyclyl is optionally substituted by one or more R⁹;
each R⁸ is independently alkyl, alkenyl, alkynyl, heteroalkyl, haloalkyl, halogen, oxo, cyano, azido, aryl, heteroaryl, cycloalkyl, heterocyclyl, OR^{A}, N(R^{C})(R^{D}), C(O)OR^{A}, C(O)R^{B}, C(O)N(R^{C})(R^{D}), or N(R^{C})C(O)R^{B}, wherein each alkyl, alkenyl, alkynyl, heteroalkyl, haloalkyl, aryl, heteroaryl, cycloalkyl, and heterocyclyl is substituted by 0-12 R¹⁰;
R^{A} is hydrogen, alkyl, alkenyl, alkynyl, heteroalkyl, haloalkyl, aryl, heteroaryl, cycloalkyl, heterocyclyl, an afibrotic compound, or a peptide, wherein each alkyl, alkenyl, alkynyl, heteroalkyl, haloalkyl, aryl, heteroaryl, cycloalkyl, and heterocyclyl is substituted by 0-12 R¹⁰;
R^{B}, R^{C}, and R^{D} are each independently alkyl, alkenyl, alkynyl, heteroalkyl, haloalkyl, halogen, aryl, heteroaryl, cycloalkyl, heterocyclyl, an afibrotic compound, or a peptide; wherein each alkyl, alkenyl, alkynyl, heteroalkyl, haloalkyl, aryl, heteroaryl, cycloalkyl, and is optionally substituted by one or more R¹⁰; or
R^{B} and R^{C} are taken together with the atoms to which they are attached to form a 3-10 membered heterocyclyl or heteroaryl ring, each of which is optionally substituted with one or more R¹⁰;
each R⁹ is independently alkyl, heteroalkyl, haloalkyl, halogen, oxo, OR^{A}, N(R^{C})(R^{D}), C(O)OR^{A}, C(O)R^{B}, C(O)N(R^{C})(R^{D}), or N(R^{C})C(O)R^{B}, wherein each alkyl, heteroalkyl, and haloalkyl is optionally substituted by one or more R¹⁰; and
each R¹⁰ is independently C₁₋₆ alkyl, halogen, oxo, cycloalkyl, or heterocyclyl.
   7. The polysaccharide polymer of any one of embodiments 1-6, wherein the saccharide monomer has a structure of Formula (I-a):

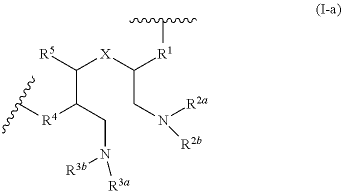

or a pharmaceutically acceptable salt thereof, wherein:
X is O, NR⁶ or S;
R¹ is absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{c})C(O)—, wherein alkylene, alkenylene, alkynylene, heteroalkylene, and haloalkylene is optionally substituted by one or more R⁸;
R^{2a}, R^{2b}, R^{3a}, and R^{3b} are each independently hydrogen, C₁₋₆ alkyl, C₁₋₆ heteroalkyl, C₁₋₆ haloalkyl, C(O)R^{B}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted by one or more R⁹, wherein at least one of R^{2a} and R^{2b} and at least one of R^{3a} and R^{3b} is not hydrogen;
each R⁴ is absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{c})C(O)—, wherein each alkylene, alkenylene, alkynylene, heteroalkylene, and is optionally substituted by one or more R¹;
R⁵ is hydrogen, C₁₋₆ alkyl, C₂₋₆ alkenyl, C₂₋₆ alkynyl, C₁₋₆ heteroalkyl, C₁₋₆ haloalkyl, N(R^{7a})(R^{7b}), OR^{A}, C(O)R^{B}, C(O)OR^{A}, C(O)N(R^{C})(R^{D}), N(R^{C})C(O)R^{B}, halogen, cyano, azido, aryl, heteroaryl, cycloalkyl, heterocyclyl, an afibrotic compound, or a peptide, wherein alkyl, alkenyl, alkynyl, heteroalkyl, haloalkyl, aryl, heteroaryl, cycloalkyl, and heterocyclyl is optionally substituted by one or more R¹;
R⁶ is hydrogen, C₁₋₆ alkyl, C₁₋₆ heteroalkyl, or C₁₋₆ haloalkyl, wherein alkyl, heteroalkyl, and haloalkyl is optionally substituted by one or more R⁹;
R^{7a} and R^{7b} are each independently hydrogen, C₁₋₆ alkyl, cycloalkyl, or heterocyclyl, wherein alkyl, cycloalkyl, or heterocyclyl is optionally substituted by one or more R⁹;
each R⁸ is independently C₁-C₆ alkyl, C₂-C₆ alkenyl, C₂-C₆ alkynyl, C₁-C₆ heteroalkyl, C₁-C₆ haloalkyl, halogen, oxo, cyano, azido, aryl, heteroaryl, cycloalkyl, heterocyclyl, OR^{A}, N(R^{C})(R^{D}), C(O)OR^{A}, C(O)R^{B}, C(O)N(R^{C})(R^{D}), or N(R^{C})C(O)R^{B}, wherein each alkyl, alkenyl, alkynyl, heteroalkyl, haloalkyl, aryl, heteroaryl, cycloalkyl, and heterocyclyl is substituted by 0-12 R¹⁰;
R^{A} is hydrogen, C₁-C₆ alkyl, C₂-C₆ alkenyl, C₂-C₆ alkynyl, C₁-C₆ heteroalkyl, C₁-C₆ haloalkyl, aryl, heteroaryl, cycloalkyl, heterocyclyl, an afibrotic compound, or a peptide, wherein each alkyl, alkenyl, alkynyl, heteroalkyl, haloalkyl, aryl, heteroaryl, cycloalkyl, and heterocyclyl is substituted by 0-12 R¹⁰;
R^{B}, R^{C}, and R^{D} are C₁-C₆ alkyl, C₂-C₆ alkenyl, C₂-C₆ alkynyl, C₁-C₆ heteroalkyl, C₁-C₆ haloalkyl, halogen, aryl, heteroaryl, cycloalkyl, heterocyclyl, an afibrotic compound, or a peptide; wherein each alkyl, alkenyl, alkynyl, heteroalkyl, haloalkyl, aryl, heteroaryl, cycloalkyl, and is optionally substituted by one or more R¹⁰; or
R^{B} and R^{C} are taken together with the atoms to which they are attached to form a 3-10 membered heterocyclyl or heteroaryl ring, each of which is optionally substituted with one or more R¹⁰;
each R⁹ is independently C₁-C₆ alkyl, C₁-C₆ heteroalkyl, C₁-C₆ haloalkyl, halogen, oxo, OR^{A}, N(R^{C})(R^{D}), C(O)OR^{A}, C(O)R^{B}, C(O)N(R^{C})(R^{D}), or N(R^{C})C(O)R^{B}, wherein each alkyl, heteroalkyl, and haloalkyl is optionally substituted by one or more R¹⁰; and
each R¹⁰ is independently C₁₋₆ alkyl, halogen, oxo, cycloalkyl, or heterocyclyl.
   8. The polysaccharide polymer of embodiments 6-7, wherein X is O.
   9. The polysaccharide polymer of any one of embodiments 6-8, wherein R¹ is OR^{A}.
   10. The polysaccharide polymer of any one of embodiments 6-8, wherein R⁵ is C(O)OR^{A} or C(O)N(R^{C})(R^{D}).
   11. The polysaccharide polymer of embodiment 10, wherein R⁵ is C(O)N(R^{C})(R^{D}), and R^{C} and R^{D} are each independently hydrogen, an afibrotic compound (e.g., an afibrotic compound provided in Table 2), or a peptide (e.g., an RGD peptide).
   12. The polysaccharide polymer of embodiment 11, wherein one of R^{C} and R is independently hydrogen and the other of R^{C} and R^{D} is independently an afibrotic compound (e.g., an afibrotic compound provided in Table 2) or a peptide (e.g., an RGD peptide).
   13. The polysaccharide polymer of any one of embodiments 6-12, wherein R² is C₁₋₆ alkyl, C₂₋₆ alkenyl, C₂₋₆ alkynyl, C₁₋₆ heteroalkyl, C₁₋₆ haloalkyl, C(O)R^{B}, aryl, heteroaryl, cycloalkyl, or heterocyclyl.
   14. The polysaccharide polymer of any one of embodiments 6-13, wherein R³ is C₁₋₆ alkyl, C₂₋₆ alkenyl, C₂₋₆ alkynyl, C₁₋₆ heteroalkyl, C₁₋₆ haloalkyl, C(O)R^{B}, aryl, heteroaryl, cycloalkyl, or heterocyclyl.
   15. The polysaccharide polymer of any one of embodiments 6-14, wherein one of R² and R³ is independently C₁₋₆ alkyl, C₁₋₆ heteroalkyl, C₁₋₆ haloalkyl, C(O)R^{B}, and the other of R² and R³ is independently hydrogen.
   16. The polysaccharide polymer of any one of embodiments 6-15, wherein R⁴ is OR^{A}. 17. The polysaccharide polymer of any one of embodiments 1-16, wherein the saccharide monomer has a structure of Formula (I-c):

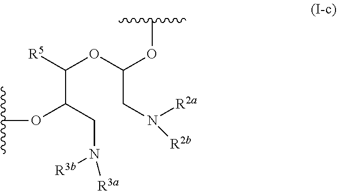

or a pharmaceutically acceptable salt thereof, wherein each of R^{2a}, R^{2b}, R^{3a}, R^{3b}, R⁵, and subvariables thereof are as defined as in embodiment 6.
18. The polysaccharide polymer of any one of embodiments 1-17, wherein the saccharide monomer has a structure of Formula (I-d):

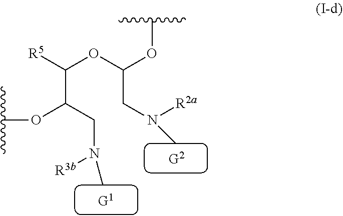

or a pharmaceutically acceptable salt thereof, wherein each of R², R^{2b}, R^{3a}, R^{3b}, R⁵, and subvariables thereof are as defined as in embodiment 6, and each of G¹ and G² is independently hydrogen, an afibrotic compound, or a peptide.
19. The polysaccharide polymer of any one of embodiments 1-18, wherein the saccharide monomer has a structure of Formula (I-e):

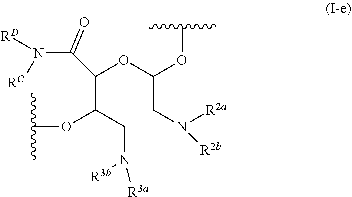

or a pharmaceutically acceptable salt thereof, wherein each of R², R^{2b}, R^{3a}, R^{3b}, R^{C}, and R^{D} and subvariables thereof are as defined as in embodiment 6.
20. The polysaccharide polymer of any one of embodiments 1-19, wherein the saccharide monomer has a structure of Formula (I-f):

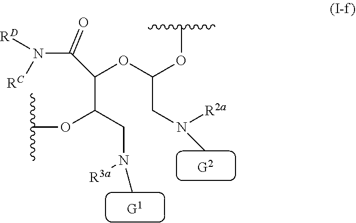

or a pharmaceutically acceptable salt thereof, wherein each of R^{2a}, R^{2b}, R^{3a}, R^{3b}, R^{C}, and R^{D} and subvariables thereof are as defined as in embodiment 6, and each of G¹ and G² is independently hydrogen, an afibrotic compound, or a peptide.
21. The polysaccharide polymer of embodiments 18-20, wherein one of G¹ and G² is independently an afibrotic compound (e.g., an afibrotic compound provided in Table 2).
22. The polysaccharide polymer of embodiments 18-20, wherein both of G¹ and G² are independently an afibrotic compound (e.g., an afibrotic compound provided in Table 2).
23. The polysaccharide polymer of any one of embodiments 1-22, wherein the saccharide monomer has a structure of Formula (I-g):

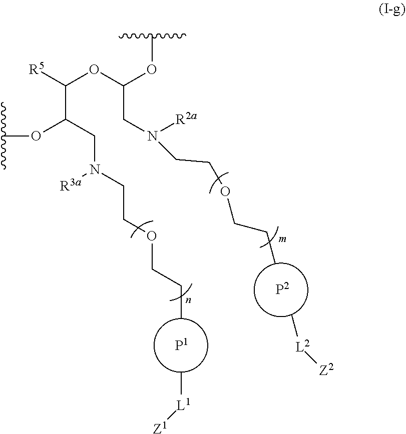

or a pharmaceutically acceptable salt thereof, wherein each of R^{2a}, R^{3a}, R⁵ and subvariables thereof are as defined as in claim 6;
P¹ and P² are each independently aryl, heteroaryl, cycloalkyl, or heterocyclyl, each of which are optionally substituted by one or more R¹²
L¹ and L² are each independently absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{C})C(O)—, wherein each alkylene, alkenylene, alkynylene, heteroalkylene, and is optionally substituted by one or more R⁸;
Z¹ and Z² are each independently hydrogen, cycloalkyl, heterocyclyl, aryl, or heteroaryl, wherein each cycloalkyl, heterocyclyl, aryl, or heteroaryl is optionally substituted by one or more R⁸
each of m and n is independently 1, 2, 3, 4, 5, 6, 7, 8, 9, 10, 11, or 12; and
R^{7a}, R⁸, and R¹² are as defined in embodiment 6.
   24. The polysaccharide polymer of any one of embodiments 1-23, wherein the saccharide monomer has a structure of Formula (I-h):

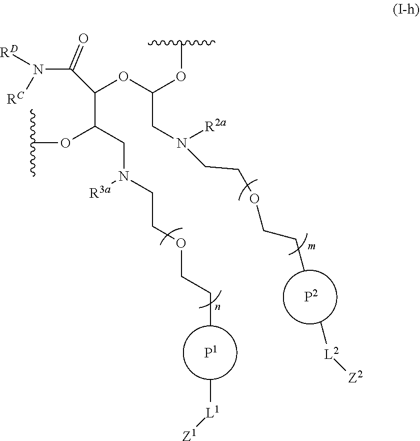

or a pharmaceutically acceptable salt thereof, wherein each of R^{2a}, R^{3a}, R^{C}, R^{D} and subvariables thereof are as defined as in embodiment 6;
P¹ and P² are each independently aryl, heteroaryl, cycloalkyl, or heterocyclyl, each of which are optionally substituted by one or more R¹²
L¹ and L² are each independently absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{c})C(O)—, wherein each alkylene, alkenylene, alkynylene, heteroalkylene, and is optionally substituted by one or more R⁸;
Z¹ and Z² are each independently hydrogen, cycloalkyl, heterocyclyl, aryl, or heteroaryl, wherein each cycloalkyl, heterocyclyl, aryl, or heteroaryl is optionally substituted by one or more R⁸
each of m and n is independently 1, 2, 3, 4, 5, 6, 7, 8, 9, 10, 11, or 12; and
R^{7a}, R⁸, and R¹² are as defined in embodiment 6.
   25. The polysaccharide polymer of embodiments 23-24, wherein P¹ and P² are each independently heteroaryl (e.g., triazolyl).
   26. The polysaccharide polymer of embodiment 25, wherein P¹ and P² are each independently

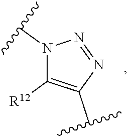

wherein R¹² is hydrogen, deuterium, C₁₋₆ alkyl, C₁₋₆ heteroalkyl, C₁₋₆ haloalkyl, or halo.
27. The polysaccharide polymer of any one of embodiments 22-25, wherein L¹ and L² are each independently absent or C₁₋₆ alkylene (e.g., —CH₂—).
28. The polysaccharide polymer of any one of embodiments 22-26, wherein Z¹ and Z² are each independently aryl, heteroaryl, or heterocyclyl.
29. The polysaccharide polymer of embodiments 22-28, wherein Z¹ and Z² are each independently heterocyclyl.
30. The polysaccharide polymer of embodiment 29, wherein Z¹ and Z² are each independently

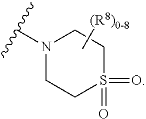

31. The polysaccharide polymer of any one of claims 1-30, wherein the afibrotic compound is selected from a moiety in Table 2 (e.g., Compound 218, Compound 219, or Compound 222).
32. The polysaccharide polymer of any one of embodiments 1-31, wherein the peptide comprises the sequence RGD.
33. The polysaccharide polymer of any one of embodiments 1-32, wherein the polysaccharide polymer is alginate.
34. The polysaccharide polymer of embodiment 33, wherein the alginate is a high guluronic acid (G) alginate or a high mannuronic acid (M) alginate.
35. The polysaccharide polymer of any one of embodiments 33-34, wherein the alginate comprises an increase in % N (as compared with unmodified polymer) of 0.1 to 10% N by weight, where % N is determined by elemental analysis.
36. The polysaccharide polymer of any one of embodiments 33-35, wherein the alginate comprises an increase in % N (as compared with unmodified polymer) of 1 to 10% N by weight, where % N is determined by elemental analysis.
37. The polysaccharide polymer of any one of embodiments 33-36, wherein the alginate comprises an increase in % N (as compared with unmodified polymer) of 2 to 8% N by weight, where % N is determined by elemental analysis and corresponds to the amount of compound of Formula (I) in the modified alginate.
38. The polysaccharide polymer of any one of embodiments 1-37, wherein the polysaccharide comprises a plurality of saccharide monomers comprising a hydroxyl-modifying agent covalently bound to a hydroxyl moiety.
39. An alginate comprising a mannuronate or guluronate monomer, wherein the mannuronate or guluronate monomer comprises a hydroxyl-modifying agent covalently bound to a hydroxyl moiety.
40. The alginate of embodiment 39, wherein the hydroxyl-modifying agent comprises an alkyl, alkenyl, alkynyl, heteroalkyl, alkoxy, haloalkyl, haloalkoxy, ester, ether, carbamate, aryl, heteroaryl, cycloalkyl, or heterocyclyl moiety.
41. The alginate of any one of embodiments 39-40, wherein the alginate comprises a monomer having a structure of Formula (I):

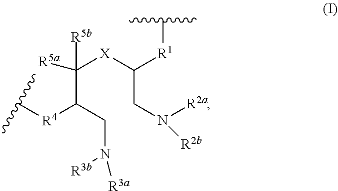

or a pharmaceutically acceptable salt thereof, wherein:
X is O, NR⁶, or S;
each of R¹ and R⁴ is independently absent, alkylene, alkenylene, alkynylene, heteroalkylene, haloalkylene, —N(R^{7a})—, —O—, —C(O)—, —C(O)O—, —C(O)N(R^{C})—, or —N(R^{c})C(O)—, wherein alkylene, alkenylene, alkynylene, heteroalkylene, and haloalkylene is optionally substituted by one or more R⁸;
R^{2a}, R^{2b}, R^{3a}, and R^{3b} are each independently hydrogen, alkyl, heteroalkyl, haloalkyl, C(O)R^{B}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted by one or more R⁹, wherein at least one of R^{2a} and R^{2b} and at least one of R^{3a} and R^{3b} is not hydrogen;
each of R^{5a} and R^{5b} is independently hydrogen, alkyl, alkenyl, alkynyl, heteroalkyl, haloalkyl, N(R^{7a})(R^{7b}), OR^{A}, C(O)R^{B}, C(O)OR^{A}, C(O)N(R^{C})(R^{D}), N(R^{C})C(O)R^{B}, halogen, cyano, azido, aryl, heteroaryl, cycloalkyl, heterocyclyl, an afibrotic compound, or a peptide, wherein alkyl, alkenyl, alkynyl, heteroalkyl, haloalkyl, aryl, heteroaryl, cycloalkyl, and heterocyclyl is optionally substituted by one or more R¹;
R⁶ is hydrogen, alkyl, heteroalkyl, or haloalkyl, wherein alkyl, heteroalkyl, and haloalkyl is optionally substituted by one or more R⁹;
R^{7a} and R^{7b} are each independently hydrogen, alkyl, cycloalkyl, or heterocyclyl, wherein alkyl, cycloalkyl, or heterocyclyl is optionally substituted by one or more R⁹;
each R⁸ is independently alkyl, alkenyl, alkynyl, heteroalkyl, haloalkyl, halogen, oxo, cyano, azido, aryl, heteroaryl, cycloalkyl, heterocyclyl, OR^{A}, N(R^{C})(R^{D}), C(O)OR^{A}, C(O)R^{B}, C(O)N(R^{C})(R^{D}), or N(R^{C})C(O)R^{B}, wherein each alkyl, alkenyl, alkynyl, heteroalkyl, haloalkyl, aryl, heteroaryl, cycloalkyl, and heterocyclyl is substituted by 0-12 R¹⁰;
R^{A} is hydrogen, alkyl, alkenyl, alkynyl, heteroalkyl, haloalkyl, aryl, heteroaryl, cycloalkyl, heterocyclyl, an afibrotic compound, or a peptide, wherein each alkyl, alkenyl, alkynyl, heteroalkyl, haloalkyl, aryl, heteroaryl, cycloalkyl, and heterocyclyl is substituted by 0-12 R¹⁰;
R^{B}, R^{C}, and R^{D} are each independently alkyl, alkenyl, alkynyl, heteroalkyl, haloalkyl, halogen, aryl, heteroaryl, cycloalkyl, heterocyclyl, an afibrotic compound, or a peptide; wherein each alkyl, alkenyl, alkynyl, heteroalkyl, haloalkyl, aryl, heteroaryl, cycloalkyl, and is optionally substituted by one or more R¹⁰; or
R^{B} and R^{C} are taken together with the atoms to which they are attached to form a 3-10 membered heterocyclyl or heteroaryl ring, each of which is optionally substituted with one or more R¹⁰;
each R⁹ is independently alkyl, heteroalkyl, haloalkyl, halogen, oxo, OR^{A}, N(R^{C})(R^{D}), C(O)OR^{A}, C(O)R^{B}, C(O)N(R^{C})(R^{D}), or N(R^{C})C(O)R^{B}, wherein each alkyl, heteroalkyl, and haloalkyl is optionally substituted by one or more R¹⁰; and
each R¹⁰ is independently C₁₋₆ alkyl, halogen, oxo, cycloalkyl, or heterocyclyl.
   42. The alginate of any one of embodiments 39-41, wherein the alginate comprises a monomer having a structure of Formula (I-a):

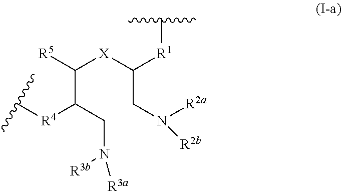

or a pharmaceutically acceptable salt thereof, wherein:
X is O, NR⁶, or S;
R¹ is absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{C})C(O)—, wherein alkylene, alkenylene, alkynylene, heteroalkylene, and haloalkylene is optionally substituted by one or more R⁸;
R^{2a}, R^{2b}, R^{3a}, and R^{3b} are each independently hydrogen, C₁₋₆ alkyl, C₁₋₆ heteroalkyl, C₁₋₆ haloalkyl, C(O)R^{B}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted by one or more R⁹, wherein at least one of R^{2a} and R^{2b} and at least one of R^{3a} and R^{3b} is not hydrogen;
each R⁴ is absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{C})C(O)—, wherein each alkylene, alkenylene, alkynylene, heteroalkylene, and is optionally substituted by one or more R⁸
R⁵ is hydrogen, C₁₋₆ alkyl, C₂₋₆ alkenyl, C₂₋₆ alkynyl, C₁₋₆ heteroalkyl, C₁₋₆ haloalkyl, N(R^{7a})(R^{7b}), OR^{A}, C(O)R^{B}, C(O)OR^{A}, C(O)N(R^{C})(R^{D}), N(R^{C})C(O)R^{B}, halogen, cyano, azido, aryl, heteroaryl, cycloalkyl, heterocyclyl, an afibrotic compound, or a peptide, wherein alkyl, alkenyl, alkynyl, heteroalkyl, haloalkyl, aryl, heteroaryl, cycloalkyl, and heterocyclyl is optionally substituted by one or more R¹;
R⁶ is hydrogen, C₁₋₆ alkyl, C₁₋₆ heteroalkyl, or C₁₋₆ haloalkyl, wherein alkyl, heteroalkyl, and haloalkyl is optionally substituted by one or more R⁹;
R^{7a} and R^{7b} are each independently hydrogen, C₁₋₆ alkyl, cycloalkyl, or heterocyclyl, wherein alkyl, cycloalkyl, or heterocyclyl is optionally substituted by one or more R⁹;
each R⁸ is independently C₁-C₆ alkyl, C₂-C₆ alkenyl, C₂-C₆ alkynyl, C₁-C₆ heteroalkyl, C₁-C₆ haloalkyl, halogen, oxo, cyano, azido, aryl, heteroaryl, cycloalkyl, heterocyclyl, OR^{A}, N(R^{C})(R^{D}), C(O)OR^{A}, C(O)R^{B}, C(O)N(R^{C})(R^{D}), or N(R^{C})C(O)R^{B}, wherein each alkyl, alkenyl, alkynyl, heteroalkyl, haloalkyl, aryl, heteroaryl, cycloalkyl, and heterocyclyl is substituted by 0-12 R¹⁰;
R^{A} is hydrogen, C₁-C₆ alkyl, C₂-C₆ alkenyl, C₂-C₆ alkynyl, C₁-C₆ heteroalkyl, C₁-C₆ haloalkyl, aryl, heteroaryl, cycloalkyl, heterocyclyl, an afibrotic compound, or a peptide, wherein each alkyl, alkenyl, alkynyl, heteroalkyl, haloalkyl, aryl, heteroaryl, cycloalkyl, and heterocyclyl is substituted by 0-12 R¹⁰;
R^{B}, R^{C}, and R^{D} are C₁-C₆ alkyl, C₂-C₆ alkenyl, C₂-C₆ alkynyl, C₁-C₆ heteroalkyl, C₁-C₆ haloalkyl, halogen, aryl, heteroaryl, cycloalkyl, heterocyclyl, an afibrotic compound, or a peptide; wherein each alkyl, alkenyl, alkynyl, heteroalkyl, haloalkyl, aryl, heteroaryl, cycloalkyl, and is optionally substituted by one or more R¹⁰; or
R^{B} and R^{C} are taken together with the atoms to which they are attached to form a 3-10 membered heterocyclyl or heteroaryl ring, each of which is optionally substituted with one or more R¹⁰;
each R⁹ is independently C₁-C₆ alkyl, C₁-C₆ heteroalkyl, C₁-C₆ haloalkyl, halogen, oxo, OR^{A}, N(R^{C})(R^{D}), C(O)OR^{A}, C(O)R^{B}, C(O)N(R^{C})(R^{D}), or N(R^{C})C(O)R^{B}, wherein each alkyl, heteroalkyl, and haloalkyl is optionally substituted by one or more R¹⁰; and
each R¹⁰ is independently C₁₋₆ alkyl, halogen, oxo, cycloalkyl, or heterocyclyl.
   43. The alginate of any one of embodiments 41-42, wherein X is O.
   44. The alginate of any one of embodiments 41-43, wherein R¹ is OR^{A}.
   45. The alginate of any one of embodiments 41-44, wherein R⁵ is C(O)OR^{A} or C(O)N(R^{C})(R^{D}).
   46. The alginate of any one of embodiments 41-45, wherein R⁵ is C(O)N(R^{C})(R^{D}), and R^{C} and R^{D} are each independently hydrogen, an afibrotic compound (e.g., an afibrotic compound provided in Table 2), or a peptide (e.g., an RGD peptide).
   47. The alginate of any one of embodiments 41-46, wherein one of R^{C} and R is independently hydrogen and the other of R^{C} and R^{D} is independently an afibrotic compound (e.g., an afibrotic compound provided in Table 2) or a peptide (e.g., an RGD peptide).
   48. The alginate of any one of embodiments 41-47, wherein R² is C₁₋₆ alkyl, C₂₋₆ alkenyl, C₂₋₆ alkynyl, C₁₋₆ heteroalkyl, C₁₋₆ haloalkyl, C(O)R^{B}, aryl, heteroaryl, cycloalkyl, or heterocyclyl.
   49. The alginate of any one of embodiments 41-48, wherein R³ is C₁₋₆ alkyl, C₂₋₆ alkenyl, C₂₋₆ alkynyl, C₁₋₆ heteroalkyl, C₁₋₆ haloalkyl, C(O)R^{B}, aryl, heteroaryl, cycloalkyl, or heterocyclyl.
   50. The alginate of any one of embodiments 41-49, wherein one of R² and R³ is independently C₁₋₆ alkyl, C₁₋₆ heteroalkyl, C₁₋₆ haloalkyl, C(O)R^{B}, and the other of R² and R³ is independently hydrogen.
   51. The alginate of any one of embodiments 41-50, wherein R⁴ is OR^{A}.
   52. The alginate of any one of embodiments 41-51, wherein the monomer has a structure of Formula (I-c):

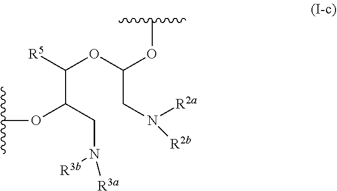

or a pharmaceutically acceptable salt thereof, wherein each of R^{2a}, R^{2b}, R^{3a}, R^{3b}, R⁵, and subvariables thereof are as defined as in embodiment 41.
53. The alginate of any one of embodiments 41-52, wherein the monomer has a structure of Formula (I-d):

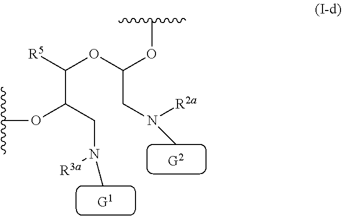

or a pharmaceutically acceptable salt thereof, wherein each of R^{2a}, R^{2b}, R^{3a}, R^{3b}, R⁵, and subvariables thereof are as defined as in embodiment 41, and each of G¹ and G² is independently hydrogen, an afibrotic compound, or a peptide.

54. The alginate of any one of embodiments 41-53, wherein the monomer has a structure of Formula (I-e):

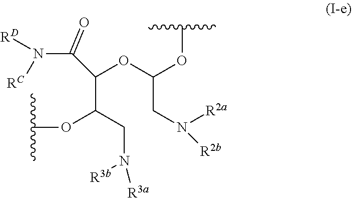

or a pharmaceutically acceptable salt thereof, wherein each of R^{2a}, R^{2b}, R^{3a}, R^{3b}, R^{C}, and R^{D} and subvariables thereof are as defined as in embodiment 41.
55. The alginate of any one of embodiments 41-54, wherein the monomer has a structure of Formula (I-f):

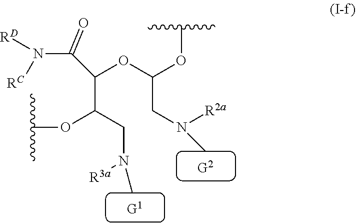

or a pharmaceutically acceptable salt thereof, wherein each of R^{2a}, R^{2b}, R^{3a}, R^{3b}, R^{C}, and R^{D} and subvariables thereof are as defined as in embodiment 41, and each of G¹ and G² is independently hydrogen, an afibrotic compound, or a peptide.
56. The alginate of embodiments 53-55, wherein one of G¹ and G² is independently an afibrotic compound.
57. The alginate of embodiments 53-55, wherein both of G¹ and G² are independently an afibrotic compound.
58. The alginate of any one of embodiments 41-55, wherein the monomer has a structure of Formula (I-g):

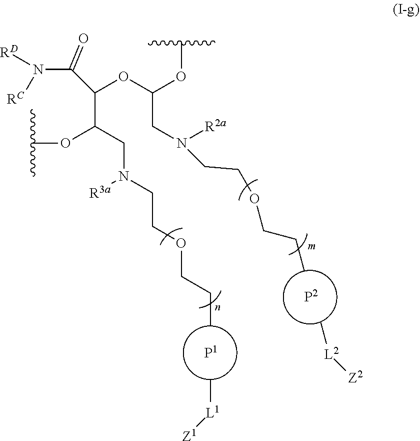

or a pharmaceutically acceptable salt thereof, wherein each of R^{2a}, R^{3a}, R⁵ and subvariables thereof are as defined as in claim 40;
P¹ and P² are each independently aryl, heteroaryl, cycloalkyl, or heterocyclyl, each of which are optionally substituted by one or more R¹²
L¹ and L² are each independently absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{c})C(O)—, wherein each alkylene, alkenylene, alkynylene, heteroalkylene, and is optionally substituted by one or more R⁸;
Z¹ and Z² are each independently hydrogen, cycloalkyl, heterocyclyl, aryl, or heteroaryl, wherein each cycloalkyl, heterocyclyl, aryl, or heteroaryl is optionally substituted by one or more R⁸
each of m and n is independently 1, 2, 3, 4, 5, 6, 7, 8, 9, 10, 11, or 12; and
R^{7a}, R⁸, and R¹² are as defined in embodiment 41.
   59. The alginate of any one of embodiments 41-58, wherein the monomer has a structure of Formula (I-h):

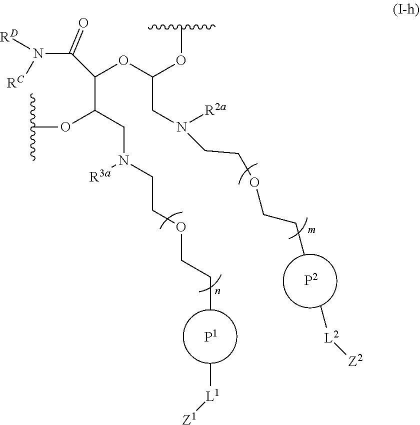

or a pharmaceutically acceptable salt thereof, wherein each of R^{2a}, R^{3a}, R^{C}, R^{D} and subvariables thereof are as defined as in embodiment 41;
P¹ and P² are each independently aryl, heteroaryl, cycloalkyl, or heterocyclyl, each of which are optionally substituted by one or more R¹²
L¹ and L² are each independently absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{c})C(O)—, wherein each alkylene, alkenylene, alkynylene, heteroalkylene, and is optionally substituted by one or more R⁸;
Z¹ and Z² are each independently hydrogen, cycloalkyl, heterocyclyl, aryl, or heteroaryl, wherein each cycloalkyl, heterocyclyl, aryl, or heteroaryl is optionally substituted by one or more R⁸;
each of m and n is independently 1, 2, 3, 4, 5, 6, 7, 8, 9, 10, 11, or 12; and
R^{7a}, R⁸, and R¹² are as defined in embodiment 41.
   60. The alginate of embodiments 58-59, wherein P¹ and P² are each independently heteroaryl (e.g., triazolyl).

61. The alginate of embodiment 60, wherein P¹ and P² are each independently

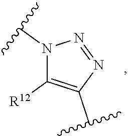

wherein R¹² is hydrogen, deuterium, C₁₋₆ alkyl, C₁₋₆ heteroalkyl, C₁₋₆ haloalkyl, or halo.
62. The alginate of any one of embodiments 60-61, wherein L¹ and L² are each independently absent or C₁₋₆ alkylene (e.g., —CH₂—).
63. The alginate of any one of embodiments 60-62, wherein Z¹ and Z² are each independently aryl, heteroaryl, or heterocyclyl.
64. The alginate of embodiment 63, wherein Z¹ and Z² are each independently heterocyclyl.
65. The alginate of embodiment 64, wherein Z¹ and Z² are each independently

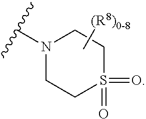

66. The alginate of any one of claims 40-65, wherein the afibrotic compound is selected from a moiety in Table 2.
67. A hydrogel comprising a polysaccharide polymer of any one of embodiments 1-40 or the alginate of any one of claims 41-66.
68. An implantable element comprising a polysaccharide polymer of any one of embodiments 1-40, an alginate of any one of embodiments 41-66, or a hydrogel of embodiment 67.
69. The implantable element of embodiment 68, further comprising a cell (e.g., an engineered cell).
70. The implantable element of embodiment 69, wherein the cell produces a therapeutic substance (e.g., an enzyme, blood clotting factor, antibody, or hormone).
71. A pharmaceutical composition comprising polysaccharide polymer of any one of embodiments 1-40, an alginate of any one of embodiments 41-66, a hydrogel of embodiment 67, or an implantable element of any one of embodiments 68-70, and a pharmaceutically acceptable excipient.
72. A method of treating a disease, disorder, or condition in a subject, e.g., a subject in need thereof, by administering to the subject an implantable element of any one of embodiment 68-70 or a pharmaceutical composition of embodiment 71, thereby treating the disease, disorder, or condition in the subject.

### Examples

In order that the invention described herein may be more fully understood, the following examples are set forth. The synthetic and biological examples described in this application are offered to illustrate the compounds, compositions, devices, and methods provided herein and are not to be construed in any way as limiting their scope.

The compounds, modified polymers, implantable elements, and compositions thereof provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Exemplary compounds, modified polymers, implantable elements, and compositions of the invention may be prepared using any of the strategies described below.

### Example 1: Synthesis of Exemplary Compounds

### General Protocols

The procedures below describe methods of preparing exemplary compounds for preparation of chemically modified implantable elements. The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Step 1: Periodate Oxidation of Alginate

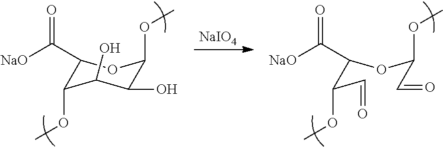

Pronova UP VLVG (Novamatrix, 2 g, ˜10.7 mmol/monomer) was dissolved in pure water (100 mL) at room temperature with vigorous stirring, then 1-propanol (10 mL) was added as a free radical scavenger. The solution was degassed for 15 min with nitrogen purging. Sodium (meta)periodate (2.3 g, 10.7 mmol) was separately dissolved in pure water (45 mL) and degassed for 15 min., then added to the reaction solution. The reaction mixture was stirred in the dark at 4° C. for 48 h, purified by a tangential flow filtration system (MWCO 10K) against saline (˜1 L), followed by pure water (˜1 L), then finally freeze-dried to afford a solid (1.72 g).

### Step 2: Reductive Amination of Oxidized Alginate

The oxidized alginate product from Step 1 (0.5 g, ˜2.7 mmol/monomer) in a 500 mL of beaker was dissolved in pure water (83 mL) to a concentration of 6 mg/ml. Methanol (11 mL) was added to a final concentration of 12% v/v. An exemplary amine to be coupled (6.6 mmol) was separately dissolved in pure water (10 mL) at room temperature. 2-Methylpyrindine borane complex (Pic-BH₃, 2.9 g, 27.1 mmol) was separately dissolved in methanol (40 mL). The amine solution was added to the oxidized alginate solution, followed by addition of Pic-BH₃ solution, resulting a suspension. The pH of the mixture was adjusted to 5.8 by addition 3 M of sodium acetate buffer solution, then 20 mL of water was added to a final alginate concentration of 3 mg/mL. The reaction mixture was vigorously stirred at room temperature for 24 hours, and floating pic-BH₃ solid was filtered out. The filtrate was purified by a tangential flow filtration system (MWCO 10K) against saline (˜1 L), followed by pure water (˜1 L), then finally freeze-dried to afford the final modified alginate as a solid. Exemplary alginates were prepared as outlined below.

Synthesis of Alginate Modified with Compound 219

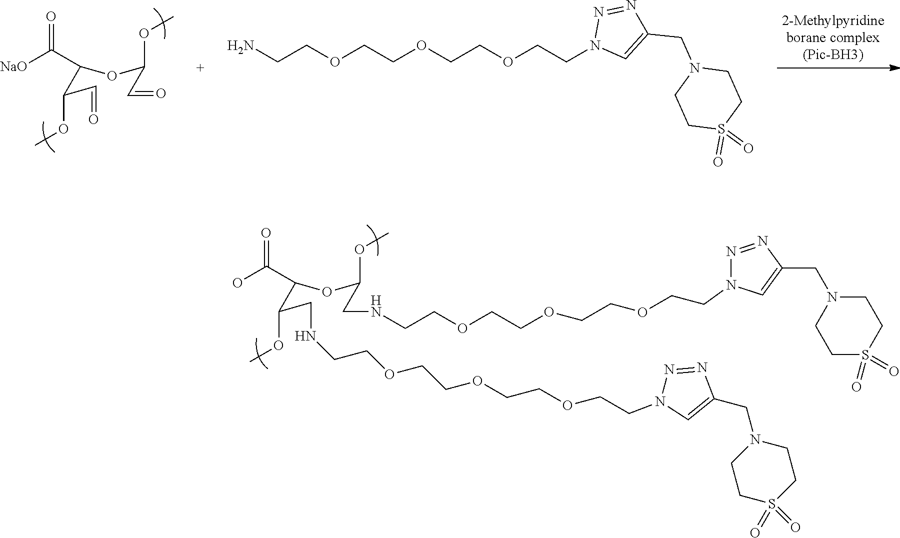

Oxidized alginate and the aminated product were prepared according to the protocols outlined in Steps 1 and 2 above. The final modified alginate containing Compound 219 was analyzed by elemental analysis indicating 7.4% of nitrogen, 37.700 carbon, and 3.200 sulfur.
Synthesis of Alginate Modified with Compound 222

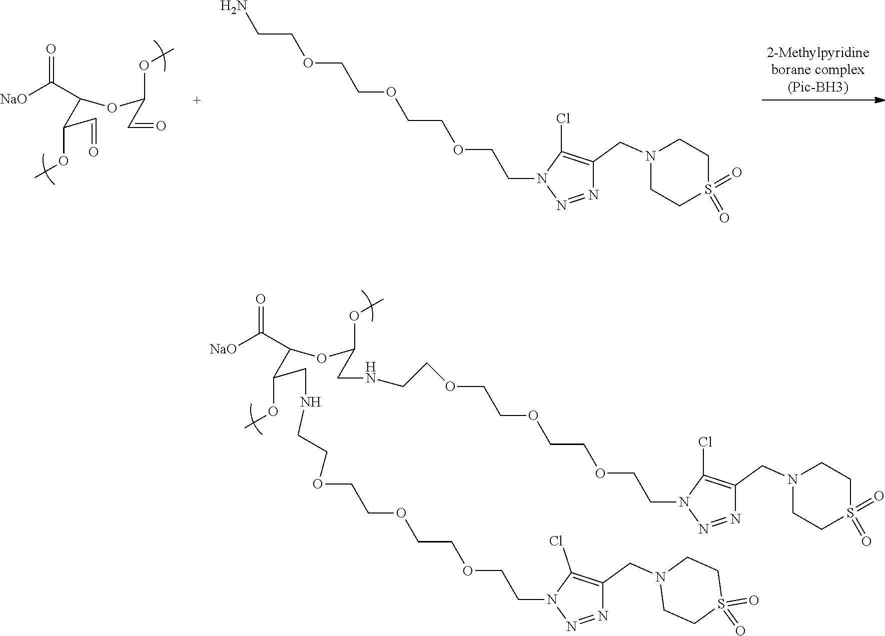

Oxidized alginate and the aminated product were prepared according to the protocols outlined in Steps 1 and 2 above. The final modified alginate analyzed by elemental analysis indicating 6.5 of nitrogen, 36.5% carbon, and 2.8% sulfur.
Synthesis of Alginate Modified with Compound 225

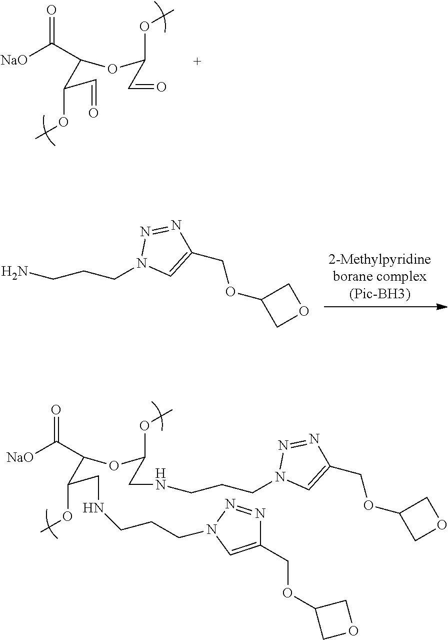

Oxidized alginate and the aminated product were prepared according to the protocols outlined in Steps 1 and 2 above. The final modified alginate was analyzed by elemental analysis indicating 10.5% of nitrogen, 41.7% carbon, and 0% sulfur.

### Example 2: Preparation of Hydrogel Capsules Comprising Modified Polysaccharide Polymers

This example describes the preparation of two compartment hydrogel capsules comprising exemplary modified polysaccharide polymers described herein. A first 5% (w/w) solution of alginate comprising Compound 219 was prepared by dissolving 0.25 g of the modified alginate in 4.75 g saline. The solution was mixed overnight until the solution was homogenous. A second alginate solution with or without an RGD peptide was also prepared (3% (w/w)) by dissolving 0.25 g RGD alginate in 8.3 g saline and mixed overnight until homogenous. 2.16 g of the 3% (w/w) RGD alginate solution was added to the 5% (w/w) modified alginate solution and mixed. The modified alginate solutions were then loaded into two separate 10 mL syringes.

A Matsusada high voltage power generator was then connected to a coaxial needle (17 gauge) sitting in a needle holder and attached to a grounding ring. The luer lock fitting on the side of the needle was attached to an 8-inch tubing extension connected to the syringe loaded with the first (5% (w/w)) alginate solution, intended to be the outer compartment. The luer lock fitting on the top of the needed is also attached to an 8-inch tubing extension connected to the syringe loaded with the second (3% (w/w)) RGD alginate, intended to be the inner compartment. Using a syring pump, the first and second alginate solutions were pumped into a glass dish containing 250 mL 20 mM BaCl₂ solution for cross-linking and hydrogel capsule formation. Cross-linked hydrogel capsules were then collected in the glass dish, washed with buffer solution (15 times), then imaged to assess morphology and sphere strength. The quality of the hydrogel capsules comprising the modified polysaccharide polymers and the afibrotic compound Compound 219 was monitored using brightfield microscopy, which revealed a smooth surface with no defects. Further testing of the stability and strength of the hydrogel capsules was carried out using texture analysis (e.g., as described in Example 7) Fracture data of the hydrogel capsules comprising modified polysaccharide polymers of comprising a monomer of Compound 219 are shown in Table 4 below.

TABLE 4
Fracture data for hydrogels modified with Compound 219

| Sphere Batch No. | Initial Fracture Strength (g) | Brittleness (mm) |
|---|---|---|
| 1 | 9.28 | 0.31 |
| 2 | 20.11 | 0.49 |
| 3 | 23.86 | 0.48 |
| 4 | 7.28 | 0.27 |
| 5 | 30.17 | 0.48 |
| 6 | 7.91 | 0.25 |
| 7 | 27.30 | 0.50 |
| 8 | 8.42 | 0.29 |
| 9 | 17.13 | 0.54 |
| 10 | 19.37 | 0.51 |
| 11 | 9.17 | 0.31 |
| 12 | 8.42 | 0.32 |
| 13 | 13.17 | 0.49 |
| 14 | 8.50 | 0.35 |
| 15 | 13.17 | 0.31 |
| 16 | 8.50 | 0.35 |
| 17 | 9.00 | 0.28 |
| 18 | 11.86 | 0.47 |
| 19 | 17.37 | 0.40 |
| 20 | 11.28 | 0.30 |

Similar fracture analysis was performed on other hydrogel capsules comprising different afibrotic compounds, specifically Compound 222 as shown in Table 5 below.

TABLE 5
Fracture data for hydrogels modified with Compound 222

| Sphere Batch No. | Initial Fracture Strength (g) | Brittleness (mm) |
|---|---|---|
| 1 | 6.35 | 0.26 |
| 2 | 6.86 | 0.273 |
| 3 | 8.05 | 0.30 |
| 4 | 6.44 | 0.25 |
| 5 | 10.03 | 0.36 |
| 6 | 7.97 | 0.31 |
| 7 | 9.65 | 0.31 |
| 8 | 8.64 | 0.29 |
| 9 | 9.61 | 0.29 |
| 10 | 6.80 | 0.27 |
| 11 | 9.93 | 0.40 |
| 12 | 9.11 | 0.35 |
| 13 | 4.57 | 0.20 |
| 14 | 6.14 | 0.24 |
| 15 | 11.61 | 0.42 |
| 16 | 8.57 | 0.28 |
| 17 | 8.55 | 0.30 |
| 18 | 5.38 | 0.22 |
| 19 | 15.04 | 0.36 |
| 20 | 9.42 | 0.28 |

### Example 3: Preparation of Exemplary Cells for Encapsulation in Hydrogel Capsules

### Engineered ARPE-19 Cells for Encapsulation as Single Cells.

ARPE-19 cells engineered to express a therapeutic agent, e.g., a blood clotting factor (e.g., a FVIII or FIX protein) may be cultured according to any method known in the art, such as according to the following protocol.

Engineered ARPE-19 cells in a 75 cm² culture flask are aspirated to remove culture medium, and the cell layer was briefly rinsed with 0.05% (w/v) trypsin/0.53 mM EDTA solution (“TrypsinEDTA”) to remove all traces of serum containing a trypsin inhibitor. 2-3 mL of TrypsinEDTA solution are added to the flask, and the cells observed under an inverted microscope until the cell layer was dispersed, usually between 5-15 minutes. To avoid clumping, cells are handled with care and hitting or shaking the flask during the dispersion period is minimized. If the cells will not detach, the flasks will be placed at 37° C. to facilitate dispersal. Once the cells are dispersed, 6-8 mL complete growth medium is added, and the cells aspirated by gentle pipetting. The cell suspension is then transferred to a centrifuge tube and spun down at approximately 125× g for 5-10 minutes to remove TrypsinEDTA. The supernatant is then discarded, and the cells re-suspended in fresh growth medium. Appropriate aliquots of cell suspension are added to new culture vessels, which is incubated at 37° C. The medium is renewed 2-3 times weekly.

### ARPE-19 Cells for Encapsulation as Clusters

Spheroid clusters of exemplary cells (e.g., engineered ARPE-19 cells) are prepared using AggreWell™ spheroid plates (STEMCELL Technologies) and the protocol outlined herein. On Day 1, rinsing solution (4 mL) is added to each plate, and the plates is spun down for 5 minutes at 3,000 RPM in a large centrifuge. The rinsing solution is removed by pipet, and 4 mL of the complete growth medium is added. The engineered ARPE-19 cells are seeded into the plates at the desired cell density and pipetted immediately to prevent aggregation, with the general rule of thumb that 3.9 million cells per well will generate 150 μm diameter clusters. The plate is spun down for 3 minutes at 800 RPM, and the plate is placed into an incubator overnight at 37° C.

On Day 2, the plate is removed from incubation. Using wide bore pipet tips, the cells are gently pipetted to dislodge the spheroid clusters. The clusters are filtered through a 40 μm or 80 μm cell strainer to remove extraneous detached single cells and then spun down in a centrifuge for 2×1 minute. The clusters are resuspended gently using wide bore pipet tips and are gently stirred to distribute them throughout the medium or another material (e.g., alginate).

Alternatively, ARPE-19 spheroids are prepared using the following protocol. On Day 1, AggreWell™ plates are removed from the packaging in a sterile tissue culture hood. 2 mL of Aggrewell™ Rinsing solution is added to each well. The plate is centrifuged at 2,000 g for 5 minutes to remove air bubbles, and the AggreWell™ Rinsing Solution is removed from the wells. Each well is rinsed with 2 mL of the complete growth medium, and 2 million engineered ARPE-19 cells in 3.9 mL of the complete growth medium is added to each well. The plate is centrifuged at 100 g for 3 minutes, then the cells are incubated the cells at 37° C. for 48 hours. On Day 3, the same protocol described above is used to dislodge the spheroid clusters. Alternatively, ARPE19 spheroids are prepared using a PBS MINI bioreactor (PBS Biotec, Inc., Camarillo CA, USA) with the following protocol. Cell culture media and 220 million ARPE19 cells are added into a PBS 0.1 L or PBS 0.5 L vessel which is then inserted into the base unit which is placed in an incubator. The PBS MINI speed adjust dial is set at 40 rpm and the vessel is incubated at 37° C. for at least 48 hours prior to collection of spheroids as described above.

### Example 4: Preparation of Hydrogel Capsules Comprising Cells

Capsules encapsulating RPE cells as single cells. Immediately before encapsulation, single ARPE-19 cells engineered to express a therapeutic protein are centrifuged at 1,400 r.p.m. for 1 min and washed with calcium-free Krebs-Henseleit (KH) Buffer (4.7 mM KCl, 25 mM HEPES, 1.2 mM KH₂PO₄, 1.2 mM MgSO₄×7H₂O, 135 mM NaCl, pH≈7.4, ≈290 mOsm). After washing, the cells are centrifuged again and all of the supernatant was aspirated. In some experiments, the cell pellet are then resuspended in a high molecular weight alginate solution (70:30) at the desired density of suspended single cells per ml alginate solution.

Prior to fabrication of one-compartment and two-compartment hydrogel capsules, buffers and alginate solutions are sterilized by filtration through a 0.2 μm filter using aseptic processes.

To prepare devices configured as two-compartment hydrogel capsules of about 1.5 mm diameter, an electrostatic droplet generator can be set up as follows: an ES series 0-100-kV, 20-watt high-voltage power generator (EQ series, Matsusada, NC, USA) will be connected to the top and bottom of a coaxial needle (inner lumen of 22 G, outer lumen of 18 G, Ramé-Hart Instrument Co., Succasunna, NJ, USA). The inner lumen will be attached to a first 5-mL syringe with luer lock, connected to a syringe pump oriented vertically. The outer lumen can be connected via a luer coupling to a second 5-mL syringe which may be connected to a second syringe pump oriented horizontally. To encapsulate cells only in the first (inner) compartment, a first alginate solution comprising the cells (as single cell suspension) may be placed in the first syringe and a second cell-free alginate solution comprising a polysaccharide polymer of Formula (I) can be placed in the second syringe. For control 2-compartment hydrogel capsules, the second (outer) compartment can be formed using an alginate solution that does not comprise a polysaccharide polymer of Formula (I). The two syringe pumps move the first and second alginate solutions from the syringes through both lumens of the coaxial needle and single droplets containing both alginate solutions are extruded from the needle into a glass dish containing a cross-linking solution. The settings of each syringe pump can be adjusted to achieve an optimal flow rate ratio for the two alginate solutions.

For fabrication of the two-compartment capsules, after extrusion of the desired volumes of alginate solutions, the alginate droplets are crosslinked for five minutes in a cross-linking solution which contained 25 mM HEPES, 20 mM BaCl₂, 0.2M mannitol, and poloxamer 188. Capsules that had fallen to the bottom of the crosslinking vessel are collected by pipetting into a conical tube. After the capsules settled in the tube, the crosslinking buffer is removed, and capsules are washed. Capsules without cells are washed four times with HEPES buffer (NaCl 15.428 g, KCl 0.70 g, MgCl2·6H₂O 0.488 g, 50 ml of HEPES (1 M) buffer solution (Gibco, Life Technologies, California, USA) in 2 liters of deionized water) and stored at 4° C. until use. Capsules encapsulating cells are washed four times in HEPES buffer, two times in 0.9% saline, and two times in culture media and stored in an incubator at 37° C.

### Example 5. In Vivo Assay of Exemplary Compounds

The afibrotic properties of exemplary compounds of the present disclosure may be interrogated by implanting hydrogel capsules prepared as described in Example 2 or 4 into the intraperitoneal (IP) space of C₅₇BL/6J mice according to the procedure below.

Preparation: Mice are prepared for surgery by being placed under anesthesia under a continuous flow of 1-4% isofluorane with oxygen at 0.5 L/min. Preoperatively, all mice may receive a 0.05-0.1 mg/kg of body weight dose of buprenorphine subcutaneously as a pre-surgical analgesic, along with 0.5 ml of 0.9% saline subcutaneously to prevent dehydration. A shaver with size #40 clipper blade will be used to remove hair to reveal an area of about 2 cm×2 cm on ventral midline of the animal abdomen. The entire shaved area is aseptically prepared with a minimum of 3 cycles of scrubbing with povidine (in an outward centrifugal direction from the center of the incision site when possible), followed by rinsing with 70% alcohol. A final skin paint with povidine is also applied. The surgical site is draped with sterile disposable paper to exclude surrounding hair from touching the surgical site, after disinfection of table top surface with 70% ethanol. Personnel will use proper PPE, gowning, surgical masks, and surgical gloves.

Surgicalprocedure: A sharp surgical blade or scissor is used to cut a 0.5-0.75 mm midline incision through the skin and the line a alba into the abdomen of the subject mice. The surgeon will attempt to keep the incision as small as possible. Flat sterile forceps are used to transfer a 0.5 mL aliquot of each capsule composition into the peritoneal cavity of each mouse (4 mice per composition). The abdominal muscle is closed by suturing with 5-0 Ethicon black silk or PDS-absorbable 5.0-6.0 monofilament absorbable thread, and the external skin layer is closed using wound clips. Blood and tissue debris are removed from the surgical instruments between procedures and the instruments are also re-sterilized between animal using a hot bead sterilizer. After the surgery, the animals are put back in the cage on a heat pad or under a heat lamp and monitored until they came out of anesthesia.

Intraoperative care: Animals are kept warm using Deltaphase isothermal pad. The animal's eyes are hydrated with sterile ophthalmic ointment during the period of surgery. Care is taken to avoid wetting the surgical site excessively to avoid hypothermia. Respiratory rate and character are monitored continuously. If vital signs are indicative of extreme pain and distress, the animal is euthanized in a carbon dioxide chamber followed by cervical dislocation.

Post-operative analysis: At 4 weeks post-implantation, the large majority of the capsules are retrieved from the mice and capsule cell numbers (one capsule in duplicate for each mouse) is measured using a CellTiter Glo® 3D Cell Viability Assay (Promega Corporation, Madison, WI USA). Briefly, one capsule per well is analyzed in duplicate and compared to a standard curve of plated cells. 100 μl of the CellTiter Glo® 3D reagent is added to the each well containing 100 μl of medium, the plate is placed onto a shaker at 400 rpm for 15 minutes and then luminescence read on a plate reader. Also, a texture analyzer is used to measure the mechanical strength (initial fracture) of the capsules in aliquots of each composition at pre-implantation and upon retrieval after the 1-month implantation period.

### Example 6: Conjugation of Additional Compounds to Polymers

The exemplary polymers may be further modified by conjugation of additional compounds to at one or more monomers at another position on the monomer, such as at a free carboxylate.

For example, compounds may be conjugated to reactive carboxylic acid groups on an alginate polymer. Any of the components capable of coupling to a carboxylic acid, such as an amine described herein, may be an appropriate partner for this coupling reaction.

An exemplary compound comprising a free amine may be conjugated to alginate using the method outlined herein. The alginate polymer is dissolved in water (30 mL/gram alginate) and treated with 2-chloro-4,6-dimethoxy-1,3,5-triazine (0.5 eq) and N-methylmorpholine (1 eq). The amine-containing compound of interest is then dissolved in acetonitrile (0.3M) and added to the alginate solution. The reaction is then warmed to 55° C. for 16 h, cooled to room temperature, concentrated via rotary evaporation, then dissolved in water. The mixture is then filtered through a bed of cyano-modified silica gel (Silicycle) and the filter cake washed with water. The resulting solution is then dialyzed (10,000 MWCO membrane) against water for 24 hours, replacing the water twice. The resulting solution was concentrated via lyophilization to afford the additionally functionalized alginate.

### Example 7: Particle Analysis

Particle preparations may be analyzed for various properties in vitro or at various times after implant into a subject, e.g. a test animal or a human patient. For example, the mechanical strength of a particle (e.g., a hydrogel capsule) may be determined after manufacture but before implantation by performing a fracture test using a texture analyzer. Mechanical testing of hydrogel capsules is performed on a TA.XT plus Texture Analyzer (Stable Micro Systems, Surrey, United Kingdom) using a 5 mm probe attached to a 5 kg load cell. Individual capsules are placed on a platform and are compressed from above by the probe at a fixed rate of 0.5 mm/sec. Contact between the probe and capsule is detected when a repulsive force of 1 g is measured. The probe continues to travel 90% of the distance between the contact height of the probe and the platform, compressing the capsule to the point of bursting. The resistance to the compressive force of the probe is measured and can be plotted as a function of probe travel (force v. displacement curve). Typically, before a capsule bursts completely, it will fracture slightly and the force exerted against the probe will decrease a small amount. An analysis macro can be programmed to detect the first time a decrease of 0.25-0.5 g occurs in the force v. displacement curve. The force applied by the probe when this occurs is termed the initial fracture force. In an embodiment, the fracture force for a capsule preparation manufactured using an apparatus described herein is the average of the initial fracture force for at least 10, 20, 30 or 40 capsules.

The average particle size in a particle preparation can be estimated by determining the average particle size in an aliquot using any analytical technique known in the art. In an embodiment, a desired number of particles (e.g., at least 10, 20 or 30) are removed from the preparation and examined by optical microscopy, e.g., by brightfield imaging.

### Example 8: Preparation and Characterization of Hydrogel Capsules Comprising Modified Polysaccharide Polymers of Compound 101

This example describes the preparation of two compartment alginate hydrogel capsules comprising an outer layer consisting of modified polysaccharide polymers described herein and an inner layer consisting of an unmodified polysaccharide polymer. The modified alginate polymers of Example 1 (0.25 g) were dissolved in 4.75 g saline. The solutions were mixed overnight until the solution was homogenous. A second alginate solution was also prepared (3% (w/w)) by dissolving 0.25 alginate in 8.3 g saline and mixed overnight until homogenous. 2.16 g of the 3% (w/w) unmodified alginate solution was added to the 5% (w/w) modified alginate solution and mixed. The modified alginate solutions were then loaded into two separate 10 mL syringes.

A Matsusada high voltage power generator was then connected to a coaxial needle (17 gauge) sitting in a needle holder and attached to a grounding ring. The luer lock fitting on the side of the needle was attached to an 8-inch tubing extension connected to the syringe loaded with the first (5% (w/w)) alginate solution, intended to be the outer compartment. The luer lock fitting on the top of the needed is also attached to an 8-inch tubing extension connected to the syringe loaded with the second (3% (w/w)) unmodified alginate, intended to be the inner compartment. Using a syringe pump, the first and second alginate solutions were pumped into a glass dish containing 250 mL 20 mM BaCl₂ solution for cross-linking and hydrogel capsule formation. Cross-linked hydrogel capsules were then collected in the glass dish, washed with buffer solution (15 times), then imaged to assess morphology and sphere strength.

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, FIGURES, or Examples but rather is as set forth in the appended claims. Those of ordinary skill in the art will appreciate that various changes and modifications to this description may be made without departing from the spirit or scope of the present invention, as defined in the following claims.

1. A polysaccharide polymer comprising a saccharide monomer, wherein
the saccharide monomer is selected from glucose, galactose, mannose, allose, altrose, talose, idose, gulose, fructose, ribose, arabinose, lyxose, xylose, rhamnose, glucuronic acid, galacturonic acid, mannuronic acid, and guluronic acid; and
the saccharide monomer has a structure of Formula (I-a): 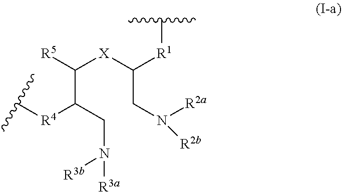
or a pharmaceutically acceptable salt thereof, wherein:
X is O, NR⁶, or S;
R¹ is absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{c})C(O)—, wherein alkylene, alkenylene, alkynylene, heteroalkylene, and haloalkylene is optionally substituted by one or more R⁸;
R^{2a}, R^{2b}, R^{3a}, and R^{3b} are each independently hydrogen, C₁₋₆ alkyl, C₁₋₆ heteroalkyl, C₁₋₆ haloalkyl, C(O)R^{B}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted by one or more R⁹, wherein at least one of R^{2a} and R^{2b} and at least one of R^{3a} and R^{3b} is not hydrogen;
each R⁴ is absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{c})C(O)—, wherein each alkylene, alkenylene, alkynylene, heteroalkylene, and is optionally substituted by one or more R⁸;
R⁵ is hydrogen, C₁₋₆ alkyl, C₂₋₆ alkenyl, C₂₋₆ alkynyl, C₁₋₆ heteroalkyl, C₁₋₆ haloalkyl, N(R^{7a})(R^{7b}), OR^{A}, C(O)R^{B}, C(O)OR^{A}, C(O)N(R^{C})(R^{D}), N(R^{C})C(O)R^{B}, halogen, cyano, azido, aryl, heteroaryl, cycloalkyl, heterocyclyl, an afibrotic compound, or a peptide, wherein alkyl, alkenyl, alkynyl, heteroalkyl, haloalkyl, aryl, heteroaryl, cycloalkyl, and heterocyclyl is optionally substituted by one or more R¹;
R⁶ is hydrogen, C₁₋₆ alkyl, C₁₋₆ heteroalkyl, or C₁₋₆ haloalkyl, wherein alkyl, heteroalkyl, and haloalkyl is optionally substituted by one or more R⁹;
R^{7a} and R^{7b} are each independently hydrogen, C₁₋₆ alkyl, cycloalkyl, or heterocyclyl, wherein alkyl, cycloalkyl, or heterocyclyl is optionally substituted by one or more R⁹;
each R⁸ is independently C₁-C₆ alkyl, C₂-C₆ alkenyl, C₂-C₆ alkynyl, C₁-C₆ heteroalkyl, C₁-C₆ haloalkyl, halogen, oxo, cyano, azido, aryl, heteroaryl, cycloalkyl, heterocyclyl, OR^{A}, N(R^{C})(R^{D}), C(O)OR^{A}, C(O)R^{B}, C(O)N(R^{C})(R^{D}), or N(R^{C})C(O)R^{B}, wherein each alkyl, alkenyl, alkynyl, heteroalkyl, haloalkyl, aryl, heteroaryl, cycloalkyl, and heterocyclyl is substituted by 0-12 R¹⁰;
R^{A} is hydrogen, C₁-C₆ alkyl, C₂-C₆ alkenyl, C₂-C₆ alkynyl, C₁-C₆ heteroalkyl, C₁-C₆ haloalkyl, aryl, heteroaryl, cycloalkyl, heterocyclyl, an afibrotic compound, or a peptide, wherein each alkyl, alkenyl, alkynyl, heteroalkyl, haloalkyl, aryl, heteroaryl, cycloalkyl, and heterocyclyl is substituted by 0-12 R¹⁰;
R^{B}, R^{C}, and R^{D} are C₁-C₆ alkyl, C₂-C₆ alkenyl, C₂-C₆ alkynyl, C₁-C₆ heteroalkyl, C₁-C₆ haloalkyl, halogen, aryl, heteroaryl, cycloalkyl, heterocyclyl, an afibrotic compound, or a peptide; wherein each alkyl, alkenyl, alkynyl, heteroalkyl, haloalkyl, aryl, heteroaryl, cycloalkyl, and is optionally substituted by one or more R¹⁰; or
R^{B} and R^{C} are taken together with the atoms to which they are attached to form a 3-10 membered heterocyclyl or heteroaryl ring, each of which is optionally substituted with one or more R¹⁰;
each R⁹ is independently C₁-C₆ alkyl, C₁-C₆ heteroalkyl, C₁-C₆ haloalkyl, halogen, oxo, OR^{A}, N(R^{C})(R^{D}), C(O)OR^{A}, C(O)R^{B}, C(O)N(R^{C})(R^{D}), or N(R^{C})C(O)R^{B}, wherein each alkyl, heteroalkyl, and haloalkyl is optionally substituted by one or more R¹⁰; and
each R¹⁰ is independently C₁₋₆ alkyl, halogen, oxo, cycloalkyl, or heterocyclyl.

2. The polysaccharide polymer of claim 1, wherein X is O.

3. The polysaccharide polymer of claim 1, wherein R¹ is OR^{A}.

4. The polysaccharide polymer of claim 1, wherein R⁵ is C(O)OR^{A} or C(O)N(R^{C})(R^{D}).

5. The polysaccharide polymer of claim 1, wherein R⁵ is C(O)N(R^{C})(R^{D}), and R^{C} and R^{D} are each independently hydrogen, an afibrotic compound (e.g., an afibrotic compound provided in Table 2), or a peptide (e.g., an RGD peptide).

6. The polysaccharide polymer of claim 5, wherein one of R^{C} and R^{D} is independently hydrogen and the other of R^{C} and R^{D} is independently an afibrotic compound (e.g., an afibrotic compound provided in Table 2) or a peptide (e.g., an RGD peptide).

7. The polysaccharide polymer of claim 1, wherein R² is C₁₋₆ alkyl, C₂₋₆ alkenyl, C₂₋₆ alkynyl, C₁₋₆ heteroalkyl, C₁₋₆ haloalkyl, C(O)R^{B}, aryl, heteroaryl, cycloalkyl, or heterocyclyl.

8. The polysaccharide polymer of claim 1, wherein R³ is C₁₋₆ alkyl, C₂₋₆ alkenyl, C₂₋₆ alkynyl, C₁₋₆ heteroalkyl, C₁₋₆ haloalkyl, C(O)R^{B}, aryl, heteroaryl, cycloalkyl, or heterocyclyl.

9. The polysaccharide polymer of claim 1, wherein one of R² and R³ is independently C₁₋₆ alkyl, C₁₋₆ heteroalkyl, C₁₋₆ haloalkyl, C(O)R^{B}, and the other of R² and R³ is independently hydrogen.

10. The polysaccharide polymer of claim 1, wherein R⁴ is OR^{A}.

11. The polysaccharide polymer of claim 1, wherein the saccharide monomer has a structure of Formula (I-c): 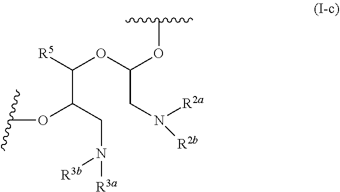
or a pharmaceutically acceptable salt thereof, wherein each of R^{2a}, R^{2b}, R^{3a}, R^{3b}, R⁵, and subvariables thereof are as defined as in claim 1.

12. The polysaccharide polymer of claim 1, wherein the saccharide monomer has a structure of Formula (I-d): 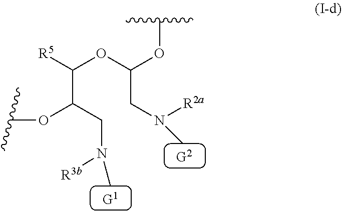
or a pharmaceutically acceptable salt thereof, wherein each of R^{2a}, R^{2b}, R^{3a}, R^{3b}, R⁵, and subvariables thereof are as defined as in claim 1, and each of G¹ and G² is independently hydrogen, an afibrotic compound (e.g., an afibrotic compound provided in Table 2), or a peptide.

13. The polysaccharide polymer of claim 1, wherein the saccharide monomer has a structure of Formula (I-e): 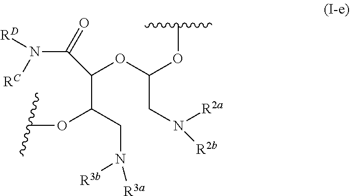
or a pharmaceutically acceptable salt thereof, wherein each of R², R^{2b}, R^{3a}, R^{3b}, R^{C}, and R^{D} and subvariables thereof are as defined as in claim 1.

14. The polysaccharide polymer of claim 1, wherein the saccharide monomer has a structure of Formula (I-f): 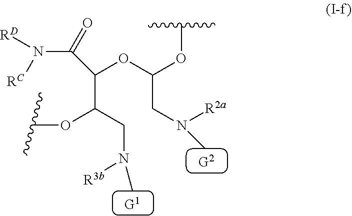
or a pharmaceutically acceptable salt thereof, wherein each of R², R^{2b}, R^{3a}, R^{3b}, R^{C}, and R^{D} and subvariables thereof are as defined as in claim 1, and each of G¹ and G² is independently hydrogen, an afibrotic compound (e.g., an afibrotic compound provided in Table 2), or a peptide.

15. The polysaccharide polymer of claim 14, wherein one of G¹ and G² is independently an afibrotic compound (e.g., an afibrotic compound provided in Table 2).

16. The polysaccharide polymer of claim 14, wherein both of G¹ and G² are independently an afibrotic compound (e.g., an afibrotic compound provided in Table 2).

17. The polysaccharide polymer of claim 1, wherein the saccharide monomer has a structure of Formula (I-g): 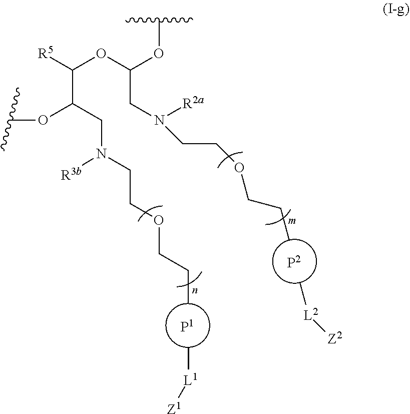
or a pharmaceutically acceptable salt thereof, wherein each of R^{2a}, R^{3a}, R⁵ and subvariables thereof are as defined as in claim 1;
P¹ and P² are each independently aryl, heteroaryl, cycloalkyl, or heterocyclyl, each of which are optionally substituted by one or more R¹²;
L¹ and L² are each independently absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{c})C(O)—, wherein each alkylene, alkenylene, alkynylene, heteroalkylene, and is optionally substituted by one or more R⁸;
Z¹ and Z² are each independently hydrogen, cycloalkyl, heterocyclyl, aryl, or heteroaryl, wherein each cycloalkyl, heterocyclyl, aryl, or heteroaryl is optionally substituted by one or more R⁸;
each of m and n is independently 1, 2, 3, 4, 5, 6, 7, 8, 9, 10, 11, or 12; and
R^{7a}, R⁸, and R¹² are as defined in claim 1.

18. The polysaccharide polymer of claim 1, wherein the saccharide monomer has a structure of Formula (I-h): 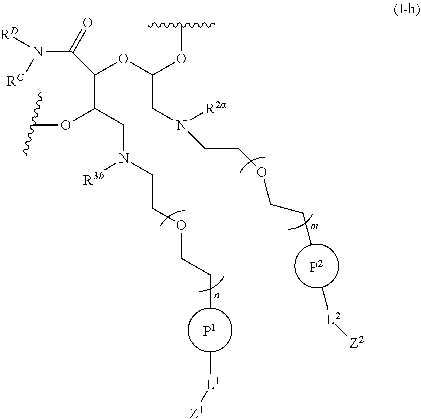
or a pharmaceutically acceptable salt thereof, wherein each of R^{2a}, R^{3a}, R^{C}, R^{D} and subvariables thereof are as defined as in claim 1;
P¹ and P² are each independently aryl, heteroaryl, cycloalkyl, or heterocyclyl, each of which are optionally substituted by one or more R¹²;
L¹ and L² are each independently absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{c})C(O)—, wherein each alkylene, alkenylene, alkynylene, heteroalkylene, and is optionally substituted by one or more R⁸;
Z¹ and Z² are each independently hydrogen, cycloalkyl, heterocyclyl, aryl, or heteroaryl, wherein each cycloalkyl, heterocyclyl, aryl, or heteroaryl is optionally substituted by one or more R⁸;
each of m and n is independently 1, 2, 3, 4, 5, 6, 7, 8, 9, 10, 11, or 12; and
R^{7a}, R⁸, and R¹² are as defined in claim 1.

19. The polysaccharide of claim 17 or 18, wherein P¹ and P² are each independently heteroaryl (e.g., triazolyl).

20. The polysaccharide polymer of claim 19, wherein P¹ and P² are each independently 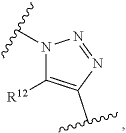
wherein R¹² is hydrogen, deuterium, C₁₋₆ alkyl, C₁₋₆ heteroalkyl, C₁₋₆ haloalkyl, or halo.

21. The polysaccharide of claim 17 or 18, wherein L¹ and L² are each independently absent or C₁₋₆ alkylene (e.g., —CH₂—).

22. The polysaccharide of claim 17 or 18, wherein Z¹ and Z² are each independently aryl, heteroaryl, or heterocyclyl.

23. The polysaccharide of claim 17 or 18, wherein Z¹ and Z² are each independently heterocyclyl.

24. The polysaccharide polymer of claim 23, wherein Z¹ and Z² are each independently

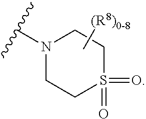

25. The polysaccharide polymer of claim 1, wherein the afibrotic compound is selected from a moiety in Table 2.

26. The polysaccharide polymer of claim 1, wherein the afibrotic compound is selected from Compound 218, Compound 219, or Compound 222.

27. The polysaccharide polymer of claim 1, wherein the peptide comprises the sequence RGD.

28. The polysaccharide polymer of claim 1, wherein the polysaccharide polymer is alginate.

29. The polysaccharide polymer of claim 29, wherein the alginate is a high guluronic acid (G) alginate or a high mannuronic acid (M) alginate.

30. An alginate comprising a monomer having a structure of Formula (I-a): 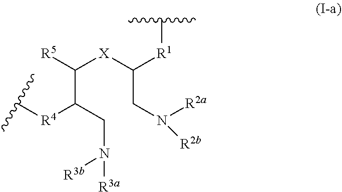
or a pharmaceutically acceptable salt thereof, wherein:
X is O, NR⁶ or S;
R¹ is absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{c})C(O)—, wherein alkylene, alkenylene, alkynylene, heteroalkylene, and haloalkylene is optionally substituted by one or more R⁸;
R^{2a}, R^{2b}, R^{3a}, and R^{3b} are each independently hydrogen, C₁₋₆ alkyl, C₁₋₆ heteroalkyl, C₁₋₆ haloalkyl, C(O)R^{B}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted by one or more R⁹, wherein at least one of R^{2a} and R^{2b} and at least one of R^{3a} and R^{3b} is not hydrogen;
each R⁴ is absent, C₁₋₆ alkylene, C₂₋₆ alkenylene, C₂₋₆ alkynylene, C₁₋₆ heteroalkylene, C₁₋₆ haloalkylene, —N(R^{7a})—, —O—, —C(O)—, —C(O)O—, —C(O)N(R^{C})—, or —N(R^{c})C(O)—, wherein each alkylene, alkenylene, alkynylene, heteroalkylene, and is optionally substituted by one or more R¹;
R⁵ is hydrogen, C₁₋₆ alkyl, C₂₋₆ alkenyl, C₂₋₆ alkynyl, C₁₋₆ heteroalkyl, C₁₋₆ haloalkyl, N(R^{7a})(R^{7b}), OR^{A}, C(O)R^{B}, C(O)OR^{A}, C(O)N(R^{C})(R^{D}), N(R^{C})C(O)R^{B}, halogen, cyano, azido, aryl, heteroaryl, cycloalkyl, heterocyclyl, an afibrotic compound, or a peptide, wherein alkyl, alkenyl, alkynyl, heteroalkyl, haloalkyl, aryl, heteroaryl, cycloalkyl, and heterocyclyl is optionally substituted by one or more R¹;
R⁶ is hydrogen, C₁₋₆ alkyl, C₁₋₆ heteroalkyl, or C₁₋₆ haloalkyl, wherein alkyl, heteroalkyl, and haloalkyl is optionally substituted by one or more R⁹;
R^{7a} and R^{7b} are each independently hydrogen, C₁₋₆ alkyl, cycloalkyl, or heterocyclyl, wherein alkyl, cycloalkyl, or heterocyclyl is optionally substituted by one or more R⁹;
each R⁸ is independently C₁-C₆ alkyl, C₂-C₆ alkenyl, C₂-C₆ alkynyl, C₁-C₆ heteroalkyl, C₁-C₆ haloalkyl, halogen, oxo, cyano, azido, aryl, heteroaryl, cycloalkyl, heterocyclyl, OR^{A}, N(R^{C})(R^{D}), C(O)OR^{A}, C(O)R^{B}, C(O)N(R^{C})(R^{D}), or N(R^{C})C(O)R^{B}, wherein each alkyl, alkenyl, alkynyl, heteroalkyl, haloalkyl, aryl, heteroaryl, cycloalkyl, and heterocyclyl is substituted by 0-12 R¹⁰;
R^{A} is hydrogen, C₁-C₆ alkyl, C₂-C₆ alkenyl, C₂-C₆ alkynyl, C₁-C₆ heteroalkyl, C₁-C₆ haloalkyl, aryl, heteroaryl, cycloalkyl, heterocyclyl, an afibrotic compound, or a peptide, wherein each alkyl, alkenyl, alkynyl, heteroalkyl, haloalkyl, aryl, heteroaryl, cycloalkyl, and heterocyclyl is substituted by 0-12 R¹⁰;
R^{B}, R^{C}, and R^{D} are C₁-C₆ alkyl, C₂-C₆ alkenyl, C₂-C₆ alkynyl, C₁-C₆ heteroalkyl, C₁-C₆ haloalkyl, halogen, aryl, heteroaryl, cycloalkyl, heterocyclyl, an afibrotic compound, or a peptide; wherein each alkyl, alkenyl, alkynyl, heteroalkyl, haloalkyl, aryl, heteroaryl, cycloalkyl, and is optionally substituted by one or more R¹⁰; or
R^{B} and R^{C} are taken together with the atoms to which they are attached to form a 3-10 membered heterocyclyl or heteroaryl ring, each of which is optionally substituted with one or more R¹⁰;
each R⁹ is independently C₁-C₆ alkyl, C₁-C₆ heteroalkyl, C₁-C₆ haloalkyl, halogen, oxo, OR^{A}, N(R^{C})(R^{D}), C(O)OR^{A}, C(O)R^{B}, C(O)N(R^{C})(R^{D}), or N(R^{C})C(O)R^{B}, wherein each alkyl, heteroalkyl, and haloalkyl is optionally substituted by one or more R¹⁰; and
each R¹⁰ is independently C₁₋₆ alkyl, halogen, oxo, cycloalkyl, or heterocyclyl.

31. The alginate of claim 30, wherein X is O.

32. The alginate of claim 30, wherein R¹ is OR^{A}.

33. The alginate of claim 30, wherein R⁵ is C(O)OR^{A} or C(O)N(R^{C})(R^{D}).

34. The alginate of claim 30, wherein R⁵ is C(O)N(R^{C})(R^{D}), and R^{C} and R^{D} are each independently hydrogen, an afibrotic compound (e.g., an afibrotic compound provided in Table 2), or a peptide (e.g., an RGD peptide).

35. The alginate of claim 30, wherein one of R^{C} and R^{D} is independently hydrogen and the other of R^{C} and R^{D} is independently an afibrotic compound (e.g., an afibrotic compound provided in Table 2) or a peptide (e.g., an RGD peptide).

36. The alginate of claim 30, wherein R² is C₁₋₆ alkyl, C₂₋₆ alkenyl, C₂₋₆ alkynyl, C₁₋₆ heteroalkyl, C₁₋₆ haloalkyl, C(O)R^{B}, aryl, heteroaryl, cycloalkyl, or heterocyclyl.

37. The alginate of claim 30, wherein R³ is C₁₋₆ alkyl, C₂₋₆ alkenyl, C₂₋₆ alkynyl, C₁₋₆ heteroalkyl, C₁₋₆ haloalkyl, C(O)R^{B}, aryl, heteroaryl, cycloalkyl, or heterocyclyl.

38. The alginate of claim 30, wherein one of R² and R³ is independently C₁₋₆ alkyl, C₁₋₆ heteroalkyl, C₁₋₆ haloalkyl, C(O)R^{B}, and the other of R² and R³ is independently hydrogen.

39. The alginate of claim 30, wherein R⁴ is OR^{A}.

40. The alginate of claim 30, wherein the monomer has a structure of Formula (I-c): 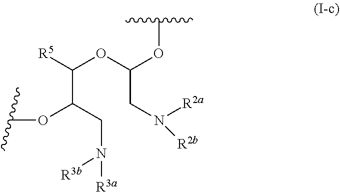
or a pharmaceutically acceptable salt thereof, wherein each of R^{2a}, R^{2b}, R^{3a}, R^{3b}, R⁵, and subvariables thereof are as defined as in claim 30.

41. The alginate of claim 30, wherein the monomer has a structure of Formula (I-d): 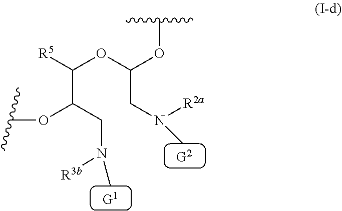
or a pharmaceutically acceptable salt thereof, wherein each of R², R^{2b}, R^{3a}, R^{3b}, R⁵, and subvariables thereof are as defined as in claim 30, and each of G¹ and G² is independently hydrogen, an afibrotic compound (e.g., as provided in Table 2), or a peptide.

42. The alginate of claim 30, wherein the monomer has a structure of Formula (I-e): 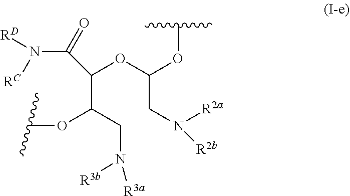
or a pharmaceutically acceptable salt thereof, wherein each of R², R^{2b}, R^{3a}, R^{3b}, R^{C}, and R^{D} and subvariables thereof are as defined as in claim 30.

43. The alginate of claim 30, wherein the monomer has a structure of Formula (I-f): 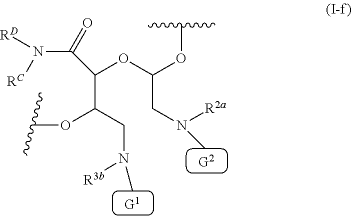
or a pharmaceutically acceptable salt thereof, wherein each of R², R^{2b}, R^{3a}, R^{3b}, R^{C}, and R^{D} and subvariables thereof are as defined as in claim 30, and each of G¹ and G² is independently hydrogen, an afibrotic compound (e.g., as provided in Table 2), or a peptide.

44. The alginate of claim 41 or 43, wherein one of G¹ and G² is independently an afibrotic compound (e.g., as provided in Table 2).

45. The alginate of claim 30, wherein the afibrotic compound is selected from a moiety in Table 2 (e.g., Compound 218, Compound 219, or Compound 222).

46. A hydrogel comprising a polysaccharide polymer of claim 1 or the alginate of claim 30.

47. An implantable element comprising a polysaccharide polymer of claim 1, an alginate of claim 30, or a hydrogel of claim 46.

48. The implantable element of claim 47, further comprising a cell (e.g., an engineered cell, e.g., as described herein).

49. The implantable element of claim 48, wherein the cell produces a therapeutic substance (e.g., a protein, e.g., an enzyme, antibody, hormone, or blood clotting factor).

50. A pharmaceutical composition comprising polysaccharide polymer of claim 1, an alginate of claim 30, a hydrogel of claim 46, or an implantable element of claim 47, and a pharmaceutically acceptable excipient.


SEQUENCE LISTING (33 sequences)

SEQ ID NO: 1 - protein, 12 aa, synthetic construct
SWELYYPLRANL

SEQ ID NO: 2 - protein, 5 aa, synthetic construct
HAVDI

SEQ ID NO: 3 - protein, 4 aa, synthetic construct
DGEA

SEQ ID NO: 4 - protein, 6 aa, synthetic construct
FYFDLR

SEQ ID NO: 5 - protein, 6 aa, synthetic construct
  SITE 3: note = 4Hyp
GFPGER

SEQ ID NO: 6 - protein, 37 aa, synthetic construct
  SITE 19: note = 4Hyp
PGPPGPPGPPGPPGPPGFPGERGPPGPPGPPGPPGPP

SEQ ID NO: 7 - protein, 4 aa, synthetic construct
VAPG

SEQ ID NO: 8-10 - skipped (3 entries)

SEQ ID NO: 11 - protein, 6 aa, synthetic construct
KQAGDV

SEQ ID NO: 12 - protein, 5 aa, synthetic construct
PHSRN

SEQ ID NO: 13 - protein, 15 aa, synthetic construct
PHSRNGGGGGGRGDS

SEQ ID NO: 14 - protein, 4 aa, synthetic construct
REDV

SEQ ID NO: 15 - protein, 5 aa, synthetic construct
IKVAV

SEQ ID NO: 16 - protein, 17 aa, synthetic construct
SRARKQAASIKVAVADR

SEQ ID NO: 17 - skipped

SEQ ID NO: 18 - protein, 6 aa, synthetic construct
KQLREQ

SEQ ID NO: 19 - protein, 5 aa, synthetic construct
YIGSR

SEQ ID NO: 20 - skipped

SEQ ID NO: 21 - protein, 7 aa, synthetic construct
SVVYGLR

SEQ ID NO: 22 - protein, 8 aa, synthetic construct
AEIDGIEL

SEQ ID NO: 23-24 - skipped (2 entries)

SEQ ID NO: 25 - protein, 4 aa, synthetic construct
VTCG

SEQ ID NO: 26 - protein, 5 aa, synthetic construct
SVTCG

SEQ ID NO: 27-28 - skipped (2 entries)

SEQ ID NO: 29 - protein, 4 aa, synthetic construct
  SITE 1: note = 17-(4-((1,1-dioxidothiomorpholino)methyl)-1H-1,2,3- triazol-1-yl)-2-(14-(4-((1,1-dioxidothiomorpholino)methyl) -1H-1,2,3-triazol-1-yl)-1-hydroxy-6,9,12-trioxa-3-azatetra decyl)-4-hydroxy-3,9,12,15-tetraoxa-6-azaheptadecanoic acid modified residue
GRGD

SEQ ID NO: 30 - protein, 6 aa, synthetic construct
  SITE 1: note = 3-hydroxy-2-(1-hydroxy-2-((4-(4-((oxetan-3-yloxy)me thyl)-1H-1,2,3-triazol-1-yl)benzyl)amino)ethoxy)-4-((4-(4- ((oxetan-3-yloxy)methyl)-1H-1,2,3-triazol-1-yl)benzyl)amin o)butanoic acid modified residue
GRGDSP

SEQ ID NO: 31 - protein, 6 aa, synthetic construct
  SITE 1: note = 17-(5-(difluoromethyl)-4-((1,1-dioxidothiomorpholin o)methyl)-1H-1,2,3-triazol-1-yl)-2-(14-(5-(difluoromethyl) -4-((1,1-dioxidothiomorpholino)methyl)-1H-1,2,3-triazol-1- yl)-1-hydroxy-6,9,12-trioxa-3-azatetradecyl)-4-hydroxy-3,9 ,12,15-tetraoxa-6-azaheptadecanoic acid modified residue
GRGDSP

SEQ ID NO: 32 - protein, 4 aa, synthetic construct
  SITE 1: note = 3-hydroxy-2-(1-hydroxy-2-((4-(4-(2-((tetrahydro-2H- pyran-2-yl)oxy)ethyl)-1H-1,2,3-triazol-1-yl)benzyl)amino)e thoxy)-4-((4-(4-(2-((tetrahydro-2H-pyran-2-yl)oxy)ethyl)-1 H-1,2,3-triazol-1-yl)benzyl)amino)butanoic acid modified residue
GRGD

SEQ ID NO: 33 - protein, 4 aa, synthetic construct
  SITE 1: note = 17-(4-((2-oxa-7-azaspiro[3.5]nonan-7-yl)methyl)-1H- 1,2,3-triazol-1-yl)-2-(14-(4-((2-oxa-7-azaspiro[3.5]nonan- 7-yl)methyl)-1H-1,2,3-triazol-1-yl)-1-hydroxy-6,9,12-triox a-3-azatetradecyl)-4-hydroxy-3,9,12,15-tetraoxa-6-azahepta decanoic acid modified residue
GRGD